(19) United States
(12) Patent Application Publication
(10) Pub. No.: US 2024/0358794 A1
(43) Pub. Date: Oct. 31, 2024

(54) COMPOSITIONS AND METHODS FOR IMPROVED TREATMENT OF DISORDERS AFFECTING THE CENTRAL NERVOUS SYSTEM

(71) Applicant: King’s College London, London (GB)

(72) Inventors: Christopher SHAW, London (GB); Youn Bok LEE, London (GB); Do Young LEE, London (GB)

(21) Appl. No.: 18/682,625

(22) PCT Filed: Aug. 10, 2022
(86) PCT No.: PCT/EP2022/072487

§ 371 Date: Feb. 9, 2024

Related U.S. Application Data
(60) Provisional application No. 63/331,614, filed on Apr. 15, 2022.
(60) Provisional application No. 63/232,053, filed on Aug. 11, 2021.

(51) Int. Cl. A61K 38/18; A61K 9/00; A61K 48/00; A61P 25/28

(52) U.S. Cl. CPC A61K 38/18; A61K 9/0019; A61K 48/0033; A61K 48/0058; A61P 25/28

(57) ABSTRACT

Described herein are methods for treating a subject having or at risk of developing a disorder affecting the central nervous system (CNS) (e.g., a neurocognitive disorder, a neuromuscular disorder, or a neurodegenerative disorder (such as FTD, AD, PD, dementia with Lewy bodies, ALS, or a related neurocognitive or motor neuron disorder) or a lysosomal storage disorder). The methods of the disclosure may include administering an adeno-associated viral (AAV) vector that expresses a therapeutic protein (e.g., whose deficiency or lack of activity is associated with the disorder or whose supplementation is likely to benefit the patient). The AAV vectors of the disclosure may be administered, e.g., in particular quantities and by way of particular routes of administration that achieve gene expression in the CNS while avoiding transduction in peripheral tissues (e.g., liver, lung, and spleen).

### FIELD OF THE INVENTION

The disclosure relates to compositions and methods for treating a disease affecting the central nervous system of a subject (e.g., a human subject).

### BACKGROUND OF THE INVENTION

Progranulin (PGRN) is a 68.5 kD glycoprotein that has long been implicated in tumorigenesis, inflammation, and repair, including growth factor signaling pathways. PGRN is expressed primarily in microglia and neurons in brain tissues where it may play a growth factor-like function. Recent discoveries have implicated PGRN in neurodegenerative disorders, particularly in frontotemporal dementia (FTD), with autosomal dominant mutations in the GRN gene having been described as underlying FTD phenotypes. PGRN has also been found in association with B-amyloid plaques in Alzheimer's disease and other amyloid-related diseases (e.g., dementia with Lewy bodies) and in association with the transactivation response element TAR DNA binding protein 43 (TDP-43), which is the main disease-related protein in patients with amyotrophic lateral sclerosis (ALS). Existing treatments for such neurodegenerative diseases, such as FTD, strive to ameliorate disease symptomology. However, therapies targeting the underlying neurodegeneration are lacking, thus underscoring the need for new therapeutic avenues.

### SUMMARY OF THE INVENTION

The present disclosure provides compositions and methods that can be used for treating disorders of the central nervous system, e.g., a neurocognitive or a neuromuscular disorder (e.g., a neurodegenerative disease) or a lysosomal storage disorder, among other disorders that adversely affect the central nervous system. Exemplary disorders that can be treated using the compositions and methods of the disclosure include frontotemporal dementia (FTD), Alzheimer's disease (AD), Parkinson's disease (PD), dementia with Lewy bodies, amyotrophic lateral sclerosis (ALS), and related neurocognitive and motor neuron disorders. Using the compositions and methods of the disclosure, a patient (e.g., a mammalian patient, such as a human patient) having a neurocognitive or neuromuscular disorder (e.g., a neurodegenerative disorder such as FTD, AD, PD, dementia with Lewy bodies, ALS, or a related neurocognitive or motor neuron disorder) or lysosomal storage disorder, among other conditions described herein, may be administered an adeno-associated viral (AAV) that contains a transgene encoding a therapeutic protein (e.g., whose deficiency or lack of activity is associated with the disorder or whose supplementation is likely to benefit the patient). Exemplary transgenes useful in conjunction with the compositions and methods of the disclosure include progranulin (PGRN), among other therapeutic proteins described herein, which may, in some embodiments, be delivered in the form of codon-optimized transgenes to further augment protein expression. The methods of use described herein, in particular, are beneficial as they avoid significant transgene expression in peripheral tissues, including, but not limited to, the liver, lung, and spleen.

In a first aspect, the disclosure provides a method of effectuating expression of a therapeutic transgene (e.g., PGRN, among the various other therapeutic transgenes described herein) in the central nervous system (CNS) of a patient while minimizing, or altogether avoiding, expression of the transgene in the patient's peripheral tissue (e.g., liver, lung, and/or spleen). The patient may be one that has a disorder affecting the central nervous system (e.g., a neurocognitive disorder, a neuromuscular disorder, or a neurodegenerative disorder (such as FTD, AD, PD, dementia with Lewy bodies, ALS, or a related neurocognitive or motor neuron disorder) or a lysosomal storage disorder), and the method includes administering to the patient an AAV vector including the therapeutic transgene. In some embodiments, the AAV vector is administered to the patient intrathalamically in an amount of from about 1×10⁹ vg/hemisphere to about 9×10¹² vg/hemisphere (e.g., about 5×10⁹ vg/hemisphere to about 1×10¹² vg/hemisphere, about 1×10¹⁰ vg/hemisphere to about 5×10¹¹ vg/hemisphere, or about 5×10¹⁰ vg/hemisphere to about 1×10¹¹ vg/hemisphere).

In another aspect, the disclosure provides a method of improving cognition, reducing neurodegeneration, and/or improving neuromuscular facility in a patient in need thereof by introducing a therapeutic transgene (e.g., PGRN, among other therapeutic transgenes described herein) into the patient's CNS. The method may minimize, or altogether avoid, expression of the transgene in the patient's peripheral tissue (e.g., liver, lung, and/or spleen). The patient may be one that has a disorder affecting the central nervous system (e.g., a neurocognitive disorder, a neuromuscular disorder, or a neurodegenerative disorder (such as FTD, AD, PD, dementia with Lewy bodies, ALS, or a related neurocognitive or motor neuron disorder) or a lysosomal storage disorder), and the method includes administering to the patient an AAV vector including the therapeutic transgene. In some embodiments, the AAV vector is administered to the patient intrathalamically in an amount of from about 1×10⁹ vg/hemisphere to about 9×10¹² vg/hemisphere (e.g., about 5×10⁹ vg/hemisphere to about 1×10¹² vg/hemisphere, about 1×10¹⁰ vg/hemisphere to about 5×10¹¹ vg/hemisphere, or about 5×10¹⁰ vg/hemisphere to about 1×10¹¹ vg/hemisphere).

In another aspect, the disclosure provides a method of treating a disorder affecting the central nervous system (e.g., a neurocognitive disorder, a neuromuscular disorder, or a neurodegenerative disorder (such as FTD, AD, PD, dementia with Lewy bodies, ALS, or a related neurocognitive or motor neuron disorder) or a lysosomal storage disorder) in a human patient in need thereof, the method including administering to the patient an AAV vector including a transgene encoding a therapeutic protein (e.g., whose deficiency or lack of activity is associated with the disorder or whose supplementation is likely to benefit the patient), such as PGRN, among the various other therapeutic proteins described herein. In some embodiments, the AAV vector is administered to the patient intrathalamically in an amount of from about 1×10⁹ vg/hemisphere to about 9×10¹² vg/hemisphere (e.g., about 5×10⁹ vg/hemisphere to about 1×10¹² vg/hemisphere, about 1×10¹⁰ vg/hemisphere to about 5×10¹¹ vg/hemisphere, or about 5×10¹⁰ vg/hemisphere to about 1×10¹¹ vg/hemisphere).

In a further aspect, the disclosure provides a method of improving cognitive function in a human patient diagnosed as having a disorder affecting the central nervous system (e.g., a neurocognitive disorder, a neuromuscular disorder, or a neurodegenerative disorder (such as FTD, AD, PD, dementia with Lewy bodies, ALS, or a related neurocognitive or motor neuron disorder) or a lysosomal storage disorder), the method including administering to the patient an AAV vector including a transgene encoding a therapeutic protein (e.g., whose deficiency or lack of activity is associated with the disorder or whose supplementation is likely to benefit the patient, such as PGRN, among the various other therapeutic proteins described herein). In some embodiments, the AAV vector is administered to the patient intrathalamically in an amount of from about 1×10⁹ vg/hemisphere to about 9×10¹² vg/hemisphere (e.g., about 5×10⁹ vg/hemisphere to about 1×10¹² vg/hemisphere, about 1×10¹⁰ vg/hemisphere to about 5×10¹¹ vg/hemisphere, or about 5×10¹⁰ vg/hemisphere to about 1×10¹¹ vg/hemisphere).

In another aspect, the disclosure provides a method of expressing, or restoring expression of, a therapeutic protein (e.g., PGRN, or another therapeutic protein described herein) in the brain (e.g., frontal cortex) of a human patient diagnosed as having a disorder affecting the central nervous system (e.g., a neurocognitive disorder, a neuromuscular disorder, or a neurodegenerative disorder (such as FTD, AD, PD, dementia with Lewy bodies, ALS, or a related neurocognitive or motor neuron disorder) or a lysosomal storage disorder), the method including administering to the patient an AAV vector including a transgene encoding a therapeutic protein (e.g., whose deficiency or lack of activity is associated with the disorder or whose supplementation is likely to benefit the patient, such as PGRN, among the various other therapeutic proteins described herein). In some embodiments, the AAV vector is administered to the patient intrathalamically in an amount of from about 1×10⁹ vg/hemisphere to about 9×10¹² vg/hemisphere (e.g., about 5×10⁹ vg/hemisphere to about 1×10¹² vg/hemisphere, about 1×10¹⁰ vg/hemisphere to about 5×10¹¹ vg/hemisphere, or about 5×10¹⁰ vg/hemisphere to about 1×10¹¹ vg/hemisphere).

In some embodiments of any of the foregoing aspects, the AAV vector is administered to the patient in an amount of from about 1×10¹⁰ vg/hemisphere to about 9×10¹² vg/hemisphere (e.g., 5×10¹⁰ vg/hemisphere to about 5×10¹¹ vg/hemisphere, or about 1×10¹¹ vg/hemisphere). For example, in some embodiments, the AAV vector is administered to the patient in an amount of about 1×10¹⁰ vg/hemisphere, 2×10¹⁰ vg/hemisphere, 3×10¹⁰ vg/hemisphere, 4×10¹⁰ vg/hemisphere, 5×10¹⁰ vg/hemisphere, 6×10¹⁰ vg/hemisphere, 7×10¹⁰ vg/hemisphere, 8×10¹⁰ vg/hemisphere, 9×10¹⁰ vg/hemisphere, 1×10¹¹ vg/hemisphere, 2×10¹¹ vg/hemisphere, 3×10¹¹ vg/hemisphere, 4×10¹¹ vg/hemisphere, 5×10¹¹ vg/hemisphere, 6×10¹¹ vg/hemisphere, 7×10¹¹ vg/hemisphere, 8×10¹¹ vg/hemisphere, 9×10¹¹ vg/hemisphere, 1×10¹² vg/hemisphere, 2×10¹² vg/hemisphere, 3×10¹² vg/hemisphere, 4×10¹² vg/hemisphere, 5×10¹² vg/hemisphere, 6×10¹² vg/hemisphere, 7×10¹² vg/hemisphere, 8×10¹² vg/hemisphere, or 9×10¹² vg/hemisphere. In some embodiments, the AAV vector is administered to the patient in an amount of from about 5×10¹⁰ vg/hemisphere to about 1×10¹¹ vg/hemisphere. In some embodiments, the AAV vector is administered to the patient in an amount of about 1×10¹⁰ vg/hemisphere. In some embodiments, the AAV vector is administered to the patient in an amount of about 5×10¹⁰ vg/hemisphere. In some embodiments, the AAV vector is administered to the patient in an amount of about 1×10¹¹ vg/hemisphere.

In some embodiments of any of the foregoing aspects, the therapeutic protein is a secreted protein. In some embodiments, the therapeutic protein is a protein listed in Table 5 herein. In some embodiments, the therapeutic protein is PGRN.

In some embodiments of any of the foregoing aspects, the disorder is a neurocognitive disorder, a neuromuscular disorder, a neurodegenerative disorder, or a lysosomal storage disorder. In some embodiments, the disorder is frontotemporal dementia (FTD), Alzheimer's disease (AD), Parkinson's disease (PD), dementia with Lewy bodies, amyotrophic lateral sclerosis (ALS), or a related neurocognitive or motor neuron disorder.

In some embodiments of any of the foregoing aspects, the AAV vector is administered to the patient in a single dose per hemisphere including the amount.

In some embodiments, the AAV vector is administered to the patient in a plurality of doses (e.g., two, three, four, five, six, seven, eight, nine, or ten) per hemisphere that, together, include the amount.

In some embodiments of any of the foregoing aspects, the transgene (e.g., transgene encoding PGRN, among other therapeutic proteins described herein) is operably linked to a promoter that is active in a neuronal cell and/or a glial cell. For example, in some embodiments, the promoter is a synapsin promoter, a tetracycline-controlled transactivator protein (tTA) promoter, a reverse tetracycline-controlled transactivator protein (rTA) promoter, a U1 promoter, a U6 promoter, a U7 promoter, a prion promoter, a phosphoglycerate kinase (PGK) promoter, a CB7 promoter, an H1 promoter, a cytomegalovirus (CMV) promoter, a CMV-chicken β-actin (CBA) promoter, a glial fibrillary acidic protein (GFAP) promoter, a calcium/calmodulin-dependent protein kinase III promoter, a tubulin alpha I promoter, a microtubulin-associated protein IB (MAP IB) promoter, a neuron-specific enolase promoter, a platelet-derived growth factor beta chain promoter, a neurofilament light chain promoter, a neuron-specific VGF gene promoter, a neuronal nuclei (NeuN) promoter, a adenomatous polyposis coli (APC) promoter, an ionized calcium-binding adapter molecule 1 (Iba-1) promoter, or a homeobox protein 9 (HB9) promoter. In some embodiments, the promoter is a synapsin promoter.

In some embodiments, the synapsin promoter has a nucleic acid sequence that is at least 85% (e.g., 85%, 86%, 87%, 88% 89%, 90%, 91%, 92%, 93%, 94%, 95%, 96%, 97%, 98%, or 99%) identical to the nucleic acid sequence of SEQ ID NO: 1. For example, in some embodiments, the synapsin promoter has a nucleic acid sequence that is at least 90% identical to the nucleic acid sequence of SEQ ID NO: 1, optionally wherein the synapsin promoter has a nucleic acid sequence that is at least 95%, 96%, 97%, 98%, or 99% identical to the nucleic acid sequence of SEQ ID NO: 1. In some embodiments, the synapsin promoter has the nucleic acid sequence of SEQ ID NO: 1.

In some embodiments of any of the foregoing aspects, the transgene encodes PGRN. The PGRN may, for example, have an amino acid sequence that is at least 85% (e.g., 85%, 86%, 87%, 88% 89%, 90%, 91%, 92%, 93%, 94%, 95%, 96%, 97%, 98%, or 99%) identical to the amino acid sequence of SEQ ID NO: 2. For example, in some embodiments, the PGRN has an amino acid sequence that is at least 90% identical to the amino acid sequence of SEQ ID NO: 2, optionally wherein the PGRN has an amino acid sequence that is at least 95%, 96%, 97%, 98%, or 99% identical to the amino acid sequence of SEQ ID NO: 2. In some embodiments, the PGRN has the amino acid sequence of SEQ ID NO: 2.

In some embodiments, the transgene (e.g., encoding PGRN) is codon-optimized. For example, in some embodiments, the transgene encoding PGRN has a nucleic acid sequence that is at least 85% (e.g., 85%, 86%, 87%, 88% 89%, 90%, 91%, 92%, 93%, 94%, 95%, 96%, 97%, 98%, or 99%) identical to the nucleic acid sequence of SEQ ID NO: 3. In some embodiments, the transgene encoding PGRN has a nucleic acid sequence that is at least 90% identical to the nucleic acid sequence of SEQ ID NO: 3, optionally wherein the transgene encoding PGRN has a nucleic acid sequence that is at least 95%, 96%, 97%, 98%, or 99% identical to the nucleic acid sequence of SEQ ID NO: 3. In some embodiments, the transgene encoding PGRN has the nucleic acid sequence of SEQ ID NO: 3.

In some embodiments, the transgene (e.g., a transgene encoding PGRN) is operably linked to a human growth hormone (hGH) intron. For example, in some embodiments, the hGH intron is an hGH intron 3. In some embodiments, the hGH intron has a nucleic acid sequence that is at least 85% (e.g., 85%, 86%, 87%, 88% 89%, 90%, 91%, 92%, 93%, 94%, 95%, 96%, 97%, 98%, or 99%) identical to the nucleic acid sequence of SEQ ID NO: 4. For example, in some embodiments, the hGH intron has a nucleic acid sequence that is at least 90% identical to the nucleic acid sequence of SEQ ID NO: 4, optionally wherein the hGH intron has a nucleic acid sequence that is at least 95%, 96%, 97%, 98%, or 99% identical to the nucleic acid sequence of SEQ ID NO: 4. In some embodiments, the hGH intron has the nucleic acid sequence of SEQ ID NO: 4.

In some embodiments, the transgene (e.g., encoding PGRN) is operably linked to a 3′ enhancer element. In some embodiments, the 3′ enhancer element has a nucleic acid sequence that is at least 85% (e.g., 85%, 86%, 87%, 88% 89%, 90%, 91%, 92%, 93%, 94%, 95%, 96%, 97%, 98%, or 99%) identical to the nucleic acid sequence of SEQ ID NO: 5. For example, in some embodiments, the 3′ enhancer element has a nucleic acid sequence that is at least 90% identical to the nucleic acid sequence of SEQ ID NO: 5, optionally wherein the 3′ enhancer element has a nucleic acid sequence that is at least 95%, 96%, 97%, 98%, or 99% identical to the nucleic acid sequence of SEQ ID NO: 5. In some embodiments, the 3′ enhancer element has the nucleic acid sequence of SEQ ID NO: 5.

In some embodiments of any of the foregoing aspects, the AAV has a nucleic acid sequence that is at least 85% (e.g., 85%, 86%, 87%, 88% 89%, 90%, 91%, 92%, 93%, 94%, 95%, 96%, 97%, 98%, or 99%) identical to the nucleic acid sequence of SEQ ID NO: 6. For example, in some embodiments, the AAV has a nucleic acid sequence that is at least 90% identical to the nucleic acid sequence of SEQ ID NO: 6. In some embodiments, the AAV has a nucleic acid sequence that is at least 95% identical to the nucleic acid sequence of SEQ ID NO: 6, optionally wherein the AAV has a nucleic acid sequence that is at least 96%, 97%, 98%, or 99% identical to the nucleic acid sequence of SEQ ID NO: 6. In some embodiments, the AAV has a nucleic acid of SEQ ID NO: 6.

In some embodiments of any of the foregoing aspects, prior to administration of the AAV vector, the patient exhibits a level of expression of the endogenous therapeutic protein (e.g., PGRN) that is from about 1% to about 40% of the level of the endogenous therapeutic protein expression level (e.g., endogenous PGRN expression level) observed in a human subject of the same age, gender, and/or body mass index that does not have a disorder affecting the central nervous system (e.g., a neurocognitive disorder, a neuromuscular disorder, or a neurodegenerative disorder (such as FTD, AD, PD, dementia with Lewy bodies, ALS, or a related neurocognitive or motor neuron disorder) or a lysosomal storage disorder).

In some embodiments, following administration of the AAV vector, the patient exhibits an increase expression of the therapeutic protein (e.g., PGRN, among other therapeutic proteins described herein) relative to a measurement of the patient's therapeutic protein expression level obtained prior to administration of the AAV vector. In some embodiments, the increase in therapeutic protein (e.g., PGRN) expression is observed in the patient's thalamus, frontal cortex, basal ganglia, parietal cortex, temporal cortex, parietal and temporal cortices, and/or cerebral spinal fluid (CSF).

In some embodiments, following administration of the AAV vector, the patient exhibits a level of therapeutic protein expression (e.g., PGRN expression) of from about 2 ng/mg to about 100 ng/mg (e.g., 3 ng/mg to about 99 ng/mg, 4 ng/mg to about 98 ng/mg, 5 ng/mg to about 97 ng/mg, 10 ng/mg to about 90 ng/mg, 20 ng/mg to about 80 ng/mg, 30 ng/mg to about 70 ng/mg, 40 ng/mg to about 60 ng/mg, or about 50 ng/mg) in the frontal cortex.

In some embodiments of any of the foregoing aspects, the AAV vector is administered to the patient in a convection-assisted manner.

In another aspect, the disclosure provides a method of treating a disorder affecting the central nervous system (e.g., a neurocognitive disorder, a neuromuscular disorder, or a neurodegenerative disorder (such as FTD, AD, PD, dementia with Lewy bodies, ALS, or a related neurocognitive or motor neuron disorder) or a lysosomal storage disorder) in a human patient in need thereof, the method including administering to the patient an AAV vector including a transgene encoding a therapeutic protein (e.g., PGRN), wherein the AAV vector is administered to the patient in an amount sufficient to achieve a level of therapeutic protein (e.g., PGRN) expression in the brain (e.g., frontal cortex) of the patient that is equivalent to a level of expression of the therapeutic protein (e.g., PGRN) observed in a human subject having a disorder affecting the central nervous system (e.g., a neurocognitive disorder, a neuromuscular disorder, or a neurodegenerative disorder (such as FTD, AD, PD, dementia with Lewy bodies, ALS, or a related neurocognitive or motor neuron disorder) or a lysosomal storage disorder) following intrathalamic administration, in an amount of from about 1×10⁹ vg/hemisphere to about 9×10¹² vg/hemisphere (e.g., 5×10¹⁰ vg/hemisphere to about 5×10¹¹ vg/hemisphere, or about 1×10¹¹ vg/hemisphere), of an AAV vector described herein (e.g., an AAV2/9 vector having the nucleic acid sequence of SEQ ID NO: 6).

In another aspect, the disclosure provides a method of improving cognitive function in a human patient diagnosed as having a disorder affecting the central nervous system (e.g., a neurocognitive disorder, a neuromuscular disorder, or a neurodegenerative disorder (such as FTD, AD, PD, dementia with Lewy bodies, ALS, or a related neurocognitive or motor neuron disorder) or a lysosomal storage disorder), the method including administering to the patient an AAV vector including a transgene encoding a therapeutic protein (e.g., PGRN), wherein the AAV vector is administered to the patient in an amount sufficient to achieve a level of expression of the therapeutic protein (e.g., PGRN) in the brain (e.g., frontal cortex) of the patient that is equivalent to a level of therapeutic protein (e.g., PGRN) expression observed in a human subject having a disorder affecting the central nervous system (e.g., a neurocognitive disorder, a neuromuscular disorder, or a neurodegenerative disorder (such as FTD, AD, PD, dementia with Lewy bodies, ALS, or a related neurocognitive or motor neuron disorder) or a lysosomal storage disorder) following intrathalamic administration, in an amount of from about 1×10⁹ vg/hemisphere to about 9×10¹² vg/hemisphere (e.g., 5×10¹⁰ vg/hemisphere to about 5×10¹¹ vg/hemisphere, or about 1×10¹¹ vg/hemisphere), of an AAV vector described herein (e.g., an AAV2/9 vector having the nucleic acid sequence of SEQ ID NO: 6).

In another aspect, the disclosure provides a method of expressing, or restoring expression of, a therapeutic protein (e.g., PGRN) in the brain (e.g., frontal cortex) of a human patient diagnosed as having a disorder affecting the central nervous system (e.g., a neurocognitive disorder, a neuromuscular disorder, or a neurodegenerative disorder (such as FTD, AD, PD, dementia with Lewy bodies, ALS, or a related neurocognitive or motor neuron disorder) or a lysosomal storage disorder), the method including administering to the patient an AAV vector including a transgene encoding a therapeutic protein (e.g., PGRN), wherein the AAV vector is administered to the patient in an amount sufficient to achieve a level of therapeutic protein expression (e.g., PGRN expression) in the brain (e.g., frontal cortex) of the patient that is equivalent to a level of therapeutic protein (e.g., PGRN) expression observed in a human subject having a disorder affecting the central nervous system (e.g., a neurocognitive disorder, a neuromuscular disorder, or a neurodegenerative disorder (such as FTD, AD, PD, dementia with Lewy bodies, ALS, or a related neurocognitive or motor neuron disorder) or a lysosomal storage disorder) following intrathalamic administration, in an amount of from about 1×10⁹ vg/hemisphere to about 9×10¹² vg/hemisphere (e.g., 5×10¹⁰ vg/hemisphere to about 5×10¹¹ vg/hemisphere, or about 1×10¹¹ vg/hemisphere) of an AAV vector described herein (e.g., an AAV2/9 vector having the nucleic acid sequence of SEQ ID NO: 6).

In another aspect, the disclosure provides a method of treating a disorder affecting the central nervous system (e.g., a neurocognitive disorder, a neuromuscular disorder, or a neurodegenerative disorder (such as FTD, AD, PD, dementia with Lewy bodies, ALS, or a related neurocognitive or motor neuron disorder) or a lysosomal storage disorder) in a human patient in need thereof, the method including administering to the patient an AAV vector including a transgene encoding a therapeutic protein (e.g., whose deficiency or lack of activity is associated with the disorder or whose supplementation is likely to benefit the patient, such as PGRN, among other therapeutic proteins described herein). In some embodiments, the AAV vector is administered to the patient in an amount sufficient to achieve a level of therapeutic protein (e.g., PGRN) expression in the brain (e.g., frontal cortex) of the patient of from about 2 ng/mg to about 8 ng/mg (e.g., 3 ng/mg to about 7 ng/mg, 4 ng/mg to about 6 ng/mg, or about 5 ng/mg), or more (e.g., about 9 ng/mg, about 10 ng/mg, about 15 ng/mg, about 20 ng/mg, about 30 ng/mg, about 40 ng/mg, about 50 ng/mg, about 60 ng/mg, about 70 ng/mg, about 80 ng/mg, about 90 ng/mg, or about 100 ng/mg).

In another aspect, the disclosure provides a method of improving cognitive function in a human patient diagnosed as having a disorder affecting the central nervous system (e.g., a neurocognitive disorder, a neuromuscular disorder, or a neurodegenerative disorder (such as FTD, AD, PD, dementia with Lewy bodies, ALS, or a related neurocognitive or motor neuron disorder) or a lysosomal storage disorder), the method including administering to the patient an AAV vector including a transgene encoding a therapeutic protein (e.g., whose deficiency or lack of activity is associated with the disorder or whose supplementation is likely to benefit the patient, such as PGRN), wherein the AAV vector is administered to the patient in an amount sufficient to achieve a level of therapeutic protein (e.g., PGRN) expression in the brain (e.g., frontal cortex) of the patient of from about 2 ng/mg to about 8 ng/mg (e.g., 3 ng/mg to about 7 ng/mg, 4 ng/mg to about 6 ng/mg, or about 5 ng/mg) or more (e.g., about 9 ng/mg, about 10 ng/mg, about 15 ng/mg, about 20 ng/mg, about 30 ng/mg, about 40 ng/mg, about 50 ng/mg, about 60 ng/mg, about 70 ng/mg, about 80 ng/mg, about 90 ng/mg, or about 100 ng/mg).

In another aspect, the disclosure provides a method of expressing, or restoring the level of expression of, a therapeutic protein (e.g., PGRN) in the brain (e.g., frontal cortex) of a human patient diagnosed as having a disorder affecting the central nervous system (e.g., a neurocognitive disorder, a neuromuscular disorder, or a neurodegenerative disorder (such as FTD, AD, PD, dementia with Lewy bodies, ALS, or a related neurocognitive or motor neuron disorder) or a lysosomal storage disorder), the method including administering to the patient an AAV vector including a transgene encoding a therapeutic protein (e.g., whose deficiency or lack of activity is associated with the disorder or whose supplementation is likely to benefit the patient, such as PGRN), wherein the AAV vector is administered to the patient in an amount sufficient to achieve a level of therapeutic protein (e.g., PGRN) expression in the brain (e.g., frontal cortex) of the patient of from about 2 ng/mg to about 8 ng/mg (e.g., 3 ng/mg to about 7 ng/mg, 4 ng/mg to about 6 ng/mg, or about 5 ng/mg) or more (e.g., about 9 ng/mg, about 10 ng/mg, about 15 ng/mg, about 20 ng/mg, about 30 ng/mg, about 40 ng/mg, about 50 ng/mg, about 60 ng/mg, about 70 ng/mg, about 80 ng/mg, about 90 ng/mg, or about 100 ng/mg).

In some embodiments of any of the foregoing aspects, the AAV vector includes capsid proteins from an AAV serotype selected from the group consisting of AAV1, AAV2, AAV3, AAV4, AAV5, AAV6, AAV7, AAV8, AAV9, AAVrh74, AAVrh.8, and AAVrh. 10.

In some embodiments, the AAV is an anterogradely-trafficked AAV or a retrogradely-trafficked AAV.

In some embodiments of any of the foregoing aspects, the AAV vector includes a 5′ inverted terminal repeat (ITR) and/or a 3′ ITR from AAV1, AAV2, AAV3, AAV4, AAV5, AAV6, AAV7, AAV8, AAV9, AAVrh74, AAVrh.8, or AAVrh. 10, optionally wherein the AAV vector includes a 5′ ITR and a 3′ ITR from AAV2. For example, in some embodiments, the AAV vector includes a 5′ ITR and a 3′ ITR from one AAV serotype and capsid proteins from a different AAV serotype.

In some embodiments, the AAV vector is an AAV2/9 vector.

In some embodiments of any of the foregoing aspects, the human patient is diagnosed as having FTD due to a mutation in the GRN gene.

In some embodiments of any of the foregoing aspects, upon administration of the AAV vector, there is either no significant increase in therapeutic protein (e.g., PGRN) expression in peripheral tissue, or any such increase in therapeutic protein (e.g., PGRN) expression is no greater than 10% (e.g., less than 9%, 8%, 7%, 6%, 5%, 4%, 3%, 2%, 1%, or 0%). In some embodiments, the peripheral tissues include, but are not limited to, the liver, lung, and/or spleen. In some embodiments, the PGRN transgene expression is calculated relative to GAPDH expression.

In another aspect, the disclosure provides a kit including an AAV vector including a transgene encoding a therapeutic protein (e.g., a therapeutic protein described herein, such as PGRN), wherein the kit further includes a package insert instructing a user of the kit to administer the AAV vector to the patient in accordance with the method of any of the foregoing aspects.

### BRIEF DESCRIPTION OF THE DRAWINGS

FIG. 1 is a map of an adeno-associated virus (AAV) encoding a codon-optimized human progranulin (hPGRN) gene (abbreviated herein as “AAV9-SYN-PGRN”). The shaded arrows and rectangles represent a nucleic acid moleucle including from 5′-to-3′ a first AAV2 inverted terminal repeat (ITR), a human synapsin (hSyn) promoter, a human growth hormone intron (hGHi3), a codon-optimized hGRN gene (PGRN-GS), a hPGRN 3′ untranslated region (hPGRN-3′UTR), a bovine growth hormone polyadenylation (poly (A)) signal, a phage-derived origin of replication (f1 ori), a Citrobacter freundii ampC B-lactamase (AmpR) promoter, a kanamycin selection gene (KanR), and a second origin of replication (ori).

FIG. 2 is a photomicrograph of progranulin (PGRN) expression across the six layers of the cortex of a sheep infused intrathalamically (ITM) with a low dose of 1×10¹⁰ vg/hemisphere of AAV9-SYN-PGRN, as described in FIG. 1. Abbreviations: NeuN, neuronal nuclei; DAPI, 4′,6-Diamidino-2-Phenylindole.

FIG. 3 is a set of graphs showing the vector genomes (vg) per μg across brain regions of biopsied tissue in sheep infused ITM with a low (1×10¹⁰ vg/hemisphere), mid (5×10¹⁰ vg/hemisphere), or high (1×10¹¹ vg/hemisphere) dose of the AAV9-SYN-PGRN, as described in FIG. 1. Abbreviations: CD/PT, caudate putamen/parietal temporal.

FIG. 4 is a set of photomicrographs of hPGRN, NeuN, and IBA1 (e.g., a marker of microglia activation) expression in the prefrontal (PF) cortex of sheep (e.g., KCL9-KCL13) infused ITM with a low (1×10¹⁰ vg/hemisphere), mid (5×10¹⁰ vg/hemisphere), or high (1×10¹¹ vg/hemisphere) dose of the AAV9-SYN-PGRN, as described in FIG. 1.

FIG. 5 is a set of graphs showing the hPGRN protein level across brain regions of sheep infused ITM with a low (1×10¹⁰ vg/hemisphere), mid (5×10¹⁰ vg/hemisphere), or high (1×10¹¹ vg/hemisphere) dose of the AAV9-SYN-PGRN, as described in FIG. 1. Abbreviations: CD/PT, caudate putamen/parietal temporal.

FIG. 6 is a set of graphs showing the hPGRN protein levels across the Frontal A cortex, Frontal B cortex, and thalamus, respectively, in sheep infused ITM with 1×10¹⁰ vg/hemisphere, 5×10¹⁰ vg/hemisphere, 1×10¹¹ vg/hemisphere, 5×10¹¹ vg/hemisphere, or 5×10¹² vg/hemisphere of the AAV9-SYN-PGRN, as described in FIG. 1.

FIG. 7 is a set of graphs showing the hPGRN protein level across brain regions of sheep infused ITM with a low (1×10¹⁰ vg/hemisphere), mid (5×10¹⁰ vg/hemisphere), or high dose (1×10¹¹ vg/hemisphere) of the AAV9-SYN-PGRN, as described in FIG. 1, normalized to the percent (%) of hPGRN protein level expression in the thalamus. Abbreviations: CD/PT, caudate putamen/parietal temporal.

FIGS. 8A and 8B are a set of graphs showing the hPGRN protein level in cerebral spinal fluid (CSF). FIG. 8A is a graph showing the hPGRN level in the CSF of patients diagnosed as having frontotemporal dementia (FTD) before and after symptom onset (presymptomatic and postsymptomatic, respectively), as compared to healthy control subjects. FIG. 8B is a graph showing the hPGRN level in the CSF of sheep four weeks post-transduction of an ITM administered low (1×10¹⁰ vg/hemisphere), mid (5×10¹⁰ vg/hemisphere), or high (1×10¹¹ vg/hemisphere) dose of the AAV9-SYN-PGRN, as described in FIG. 1.

FIGS. 9A and 9B are a set of graphs showing the hPGRN protein level in serum. FIG. 9A is a graph showing the hPGRN level in the serum of patients diagnosed as having FTD before and after symptom onset (presymptomatic and postsymptomatic, respectively), as compared to healthy control subjects. FIG. 9B is a graph showing the hPGRN level in the serum of sheep one week pre-transduction or four weeks post-transduction of an ITM administered low (1×10¹⁰ vg/hemisphere), mid (5×10¹⁰ vg/hemisphere), or high dose (1×10¹¹ vg/hemisphere) of the AAV9-SYN-PGRN, as described in FIG. 1.

FIG. 10 is a set of photomicrographs of the PF cortex and thalamus of hematoxylin-stained sections of sheep (e.g., KCL9-KCL13) infused with a 1×10¹⁰ vg/hemisphere, 5×10¹⁰ vg/hemisphere, or 1×10¹¹ vg/hemisphere dose of the AAV9-SYN-PGRN, as described in FIG. 1.

FIG. 11 is a set of graphs showing the hPGRN protein level normalized to vg/ug across brain regions of sheep ITM transduced with 1×10¹⁰ vg/hemisphere, 5×10¹⁰ vg/hemisphere, or 1×10¹¹ vg/hemisphere of the AAV9-SYN-PGRN, as described in FIG. 1.

FIG. 12 is a set of photomicrographs of hPGRN expression in the cerebellum of sheep transduced intra cisterna magna (ICM) with 1×10¹³ of the AAV9-SYN-PGRN, as described in FIG. 1 or an AAV9 or AAV1 vector expressing a transgene encoding PGRN, respectively (AAV9-CB7-PGRN and AAV1-CB7-PGRN). Panels A-C are merged images of PGRN, NeuN and DAPI. Panels D-F indicate tissue stained with anti-hPGRN antibody alone. Panels G-I indicate tissue stained with anti-NeuN antibody alone. Abbreviations: SYN: Synapsin; CB7, chicken β-actin promoter with a cytomegalovirus enhancer.

FIG. 13 is a set of photomicrographs of PGRN expression in the PF cortex and thalamus, respectively, of sheep infused ITM or ICM with 1×10¹⁰ vg/hemisphere or 1×10¹³ of AAV9-SYN-PGRN, as described in FIG. 1, or an AAV9 or AAV1 vector expressing a transgene encoding hPGRN, respectively (AAV9-CB7-PGRN and AAV1-CB7-PGRN). Abbreviations: CB7, chicken β-actin promoter with a cytomegalovirus enhancer.

FIG. 14 is a set of graphs showing the vg/μg across regions of biopsied brain tissue across sheep (e.g., KCL-9-KCL-13) transduced ITM with 1×10¹⁰ vg/hemisphere, 5×10¹⁰ vg/hemisphere, or 1 ×10¹¹ vg/hemisphere or ICM with 1×10¹³ vg/animal of AAV9-SYN-PGRN, AAV1-CB7-PGRN, or AAV9-CB7-PGRN, as described in FIG. 12. Shading intensity indicates vg expression level.

FIG. 15 is a set of graphs showing the hPGRN expression across brain regions across sheep (e.g., KCL-9-KCL-13) transduced ITM with 1×10¹⁰ vg/hemisphere, 5×10¹⁰ vg/hemisphere, 1×10¹¹, or ICM with 1×10¹³ of AAV1-CB7-PGRN, AAV9-CB7-PGRN, or AAV9-SYN-PGRN, respectively, as described in FIG. 12. Shading intensity indicates hPGRN expression level.

FIG. 16 is a graph showing the hPGRN expression across cortical brain regions in sheep transduced ITM with 5×10¹⁰ vg/hemisphere of AAV9-SYN-PGRN or ICM with 1×10¹³ vg/animal of AAV9-SYN-PGRN, AAV1-CB7-PGRN, or AAV9-CB7-PGRN, as described in FIG. 12.

FIG. 17 is a set of graphs showing the hPGRN expression in the Frontal A cortex of sheep transduced ITM with 1×10¹⁰ vg/hemisphere, 5×10¹⁰ vg/hemisphere, or 1×10¹¹ vg/hemisphere of AAV9-SYN-PGRN, or ICM with 1×10¹³ vg/animal of AAV9-SYN-PGRN, AAV1-CB7-PGRN, or AAV9-CB7-PGRN, as described in FIG. 12.

FIG. 18 is a set of graphs showing the hPGRN expression in the Frontal B cortex of sheep transduced ITM or ICM with 1×10¹⁰ vg/hemisphere, 5×10¹⁰ vg/hemisphere, or 1×10¹¹ vg/hemisphere of AAV9-SYN-PGRN, or ICM with 1×10¹³ vg/animal of AAV9-SYN-PGRN, AAV1-CB7-PGRN, or AAV9-CB7-PGRN, as described in FIG. 12.

FIG. 19 is a set of photomicrographs of PGRN and the lipofuscinosis level, as measured by the lipofuscinosis marker Subunit C Mitochondrial ATP Synthase (SCMAS), in the thalamus of GRN^{-/-}mice infused ITM with a very low dose (2.3×10⁷ vg/hemisphere e.g., equivalent to sheep dose of 1×10¹⁰ vg/hemisphere), a low dose (1.1×10⁸ vg/hemisphere e.g., equivalent to sheep dose of 5×10¹⁰ vg/hemisphere), a mid-dose (2.3×10⁸ vg/hemisphere e.g., equivalent to sheep dose of 1×10¹¹ vg/hemisphere), or a high dose (2.3×10⁹ vg/hemisphere e.g., equivalent to sheep dose of 1×10¹² vg/hemisphere) of AAV9-SYN-PGRN. The PBS injection was used as a control for SCMAS. Panels A-E are images with the PGRN and SCMAS merged. Panels F-J indicate tissue stained with an anti-hPGRN antibody alone. Panels K-O indicate tissue stained with anti-SCMAS antibody alone.

FIG. 20 is a graph showing that lipofuscinosis significantly reduced by AAV—SYN-PGRN administration in the brain of GRN/mice. Lipofuscinosis was quantified by SCMAS-positive granules in GRN″ mice infused ITM with AAV—SYN-PGRN in a very low dose (2.3×10⁷ vg/hemisphere e.g., equivalent to sheep dose of 1×10¹⁰ vg/hemisphere), a low dose (1.1×10⁸ vg/hemisphere e.g., equivalent to sheep dose of 5×10¹⁰ vg/hemisphere), a mid-dose (2.3×10⁸ vg/hemisphere e.g., equivalent to sheep dose of 1×10¹¹ vg/hemisphere), or a high dose (2.3×10⁹ vg/hemisphere e.g., equivalent to sheep dose of 1×10¹² vg/hemisphere), as compared to wild-type (WT) controls or a GRN1-home cage control (“20w Hom” e.g., an untreated mouse that spent 20 weeks in the home cage). Abbreviations: ns, not-significant.

FIG. 21 is a graph showing vector biodistribution in sheep livers 4 weeks post ITM administration of AAV9-SYN-PGRN vector in the brain of sheep. Six sites per sheep liver were biopsied, and two sheep per vector dose were administered the vector.

FIG. 22 is a graph demonstrating that minimal to no significant increase in PGRN expression levels is observed in the bloodstream following ITM administration of an AAV9-PGRN vector. Data were obtained from single bilateral ITM injection of AAV9.PGRN (Low-dose: 2.5×10¹⁰ vg/hemisphere; High-dose: 2.5×10¹¹ vg/hemisphere) in cynomolgus monkeys using Convection Enhanced Delivery. Blood samples were taken at Day 0, Week 2, 4, 8, and 12 and analyzed for PGRN protein by way of ELISA. (Note: 2.5×10¹¹ vg/hemisphere in non-human primates (e.g., cynomolgus monkeys) is equivalent to 4×10¹² vg/hemisphere in humans.)

FIG. 23 is a table demonstrating that no significant expression of PGRN is observed in non-nervous tissue following ITM administration of an AAV9-PGRN vector. Data were obtained from single bilateral ITM injection of AAV9.PGRN ((Low-dose: 2.5×10¹⁰ vg/hemisphere; High-dose: 2.5×10¹¹ vg/hemisphere) in cynomolgus monkeys using Convection Enhanced Delivery. Terminal biopsies at week 12 were taken from key non-nervous system organs and assayed for levels of hPGRN RNA by qPCR. (Note: 2.5×10¹¹ vg/hemisphere in non-human primates (e.g., cynomolgus monkeys) is equivalent to 4×10¹² vg/hemisphere in humans.)

### DEFINITIONS

As used herein, the term “about” refers to a value that is within 10% above or below the value being described.

As used herein, the term “adeno-associated virus” (AAV), includes but is not limited to, AAV type 1, AAV type 2, AAV type 3 (including types 3A and 3B), AAV type 4, AAV type 5, AAV type 6, AAV type 7, AAV type 8, AAV type 9, AAV type 10, AAV type 11, AAV type 12, AAV type 13, snake AAV, avian AAV, bovine AAV, canine AAV, equine AAV, ovine AAV, goat AAV, shrimp AAV, and any other AAV now known or later discovered. See, e.g., Fields et al. Virology, 4th ed. Lippincott-Raven Publishers, Philadelphia, 1996. Additional AAV serotypes and clades have been identified recently. (See, e.g., Gao et al. J. Virol. 78:6381 (2004); Moris et al. Virol. 33:375 (2004). The genomic sequences of various serotypes of AAV, as well as the sequences of the native ITRs, Rep proteins, and capsid subunits are known in the art. Such sequences may be found in the literature or in public databases such as GenBank. See, e.g., GenBank Accession Numbers NC-002077, NC-001401, NC-001729, NC-001863, NC-001829, NC-001862, NC-000883, NC-001701, NC-001510, NC-006152, NC-006261, AF063497, U89790, AF043303, AF028705, AF028704, J02275, J01901, J02275, X01457, AF288061, AH009962, AY028226, AY028223, AY631966, AX753250, EU285562, NC-001358, NC-001540, AF513851, AF513852 and AY530579; the disclosures of which are incorporated by reference herein for teaching AAV nucleic acid and amino acid sequences. See also, e.g., Bantel-Schaal et al. J. Virol. 73:939 (1999); Chiorini et al. J. Virol. 71:6823 (1997); Chiorini et al. J. Virol. 73:1309 (1999); Gao et al. Proc. Nat. Acad. Sci. USA 99:11854 (2002); Moris et al. Virol. 33:375 (2004); Muramatsu et al. Virol. 221:208 (1996); Ruffing et al. J. Gen. Virol. 75:3385 (1994); Rutledge et al. J. Virol. 72:309 (1998); Schmidt et al. J. Virol. 82:8911 (2008); Shade et al. J. Virol. 58:921 (1986); Srivastava et al. J. Virol. 45:555 (1983); Xiao et al. J. Virol. 73:3994 (1999); WO 00/28061, WO 99/61601, WO 98/11244; and U.S. Pat. No. 6,156,303; the disclosures of which are incorporated by reference herein for teaching AAV nucleic acid and amino acid sequences. As used herein, the term “AAV” encompasses an anterogradely-trafficked AAV and/or a retrogradely-trafficked AAV.

As used herein, the terms “amyotrophic lateral sclerosis” and “ALS”, also called Lou Gehrig's disease, refer to a fatal disease affecting motor neurons of the cortex, brain stem and spinal cord. In the context of the present invention the term “ALS” includes the spectrum of neurodegenerative disorders known under the names of Classical (Charcot's) ALS, Lou Gehrig's disease, motor neuron disease (MND), progressive bulbar palsy (PBP), progressive muscular atrophy (PMA), primary lateral sclerosis (PLS), bulbar onset ALS, spinal onset ALS and ALS with multi-system involvement (Wijesekera L C and Leigh P N. Amyotrophic lateral sclerosis. Orphanet J. Rare Dis. 2009, 4:3).

As used herein, “Alzheimer's disease” and “AD” refer to a late-onset neurodegenerative disorder presenting as cognitive decline, loss of short- and long-term memory, attention deficits, language-specific problems, disorientation, impulse control, social withdrawal, anhedonia, and other symptoms. Brain tissue of AD patients exhibits neuropathological features such as extracellular aggregates of amyloid-β protein and neurofibrillary tangles of hyperphosphorylated microtubule-associated tau proteins. Accumulation of these aggregates is associated with neuronal loss and atrophy in a number of brain regions including the frontal, temporal, and parietal lobes of the cerebral cortex as well as subcortical structures like the basal forebrain cholinergic system and the locus coeruleus within the brainstem. AD is also associated with increased neuroinflammation characterized by reactive gliosis and elevated levels of pro-inflammatory cytokines.

A “capsid protein” as used herein refers to any of the AAV capsid proteins that are components of AAV viral particles, including AAV8 and AAV9.

The term “codon” as used herein refers to any group of three consecutive nucleotide bases in a given messenger RNA molecule, or coding strand of DNA, that specifies a particular amino acid or a starting or stopping signal for translation. The term codon also refers to base triplets in a DNA strand.

As used herein, “codon optimization” refers a process of modifying a nucleic acid sequence in accordance with the principle that the frequency of occurrence of synonymous codons (e.g., codons that code for the same amino acid) in coding DNA is biased in different species. Such codon degeneracy allows an identical polypeptide to be encoded by a variety of nucleotide sequences. Sequences modified in this way are referred to herein as “codon-optimized.” This process may be performed on any of the sequences described in this specification to enhance expression or stability. Codon optimization may be performed by any manner known in the art, such as, for example, that described in, e.g., U.S. Pat. Nos. 7,561,972, 7,561,973, and 7,888,112, each of which is incorporated herein by reference in its entirety. The sequence surrounding the translational start site can be converted to a consensus Kozak sequence according to known methods. See, e.g., Kozak et al, Nucleic Acids Res. 15 (20): 8125-8148, incorporated herein by reference in its entirety. Multiple stop codons can be incorporated.

Throughout this specification and claims, the word “comprise,” or variations such as “comprises” or “comprising,” will be understood to imply the inclusion of a stated integer or group of integers but not the exclusion of any other integer or group of integers.

As used herein, the terms “conservative mutation,” “conservative substitution,” and “conservative amino acid substitution” refer to a substitution of one or more amino acids for one or more different amino acids that exhibit similar physicochemical properties, such as polarity, electrostatic charge, and steric volume. These properties are summarized for each of the twenty naturally occurring amino acids in Table 1, below.

TABLE 1
Representative physicochemical properties of naturally occurring amino acids

| | | | | Electrostatic | |
|---|---|---|---|---|---|
| | | | Side- | character at | |
| | 3 Letter | 1 Letter | chain | physiological pH | Steric |
| Amino Acid | Code | Code | Polarity | (7.4) | Volume^{†} |
| Alanine | Ala | A | nonpolar | neutral | small |
| Arginine | Arg | R | polar | cationic | large |
| Asparagine | Asn | N | polar | neutral | intermediate |
| Aspartic acid | Asp | D | polar | anionic | intermediate |
| Cysteine | Cys | C | nonpolar | neutral | intermediate |
| Glutamic acid | Glu | E | polar | anionic | intermediate |
| Glutamine | Gln | Q | polar | neutral | intermediate |
| Glycine | Gly | G | nonpolar | neutral | small |
| Histidine | His | H | polar | Both neutral and | large |
| | | | | cationic forms in | |
| | | | | equilibrium at pH 7.4 | |
| Isoleucine | Ile | I | nonpolar | neutral | large |
| Leucine | Leu | L | nonpolar | neutral | large |
| Lysine | Lys | K | polar | cationic | large |
| Methionine | Met | M | nonpolar | neutral | large |
| Phenylalanine | Phe | F | nonpolar | neutral | large |
| Proline | Pro | P | non- | neutral | intermediate |
| | | | polar | | |
| Serine | Ser | S | polar | neutral | small |
| Threonine | Thr | T | polar | neutral | intermediate |
| Tryptophan | Trp | W | nonpolar | neutral | bulky |
| Tyrosine | Tyr | Y | polar | neutral | large |
| Valine | Val | V | nonpolar | neutral | intermediate |

^{†}based on volume in A³: 50-100 is small, 100-150 is intermediate, 150-200 is large, and >200 is bulky

From this table it is appreciated that the conservative amino acid families include, e.g., (i) G, A, V, L, I, P, and M; (ii) D and E; (iii) C, S and T; (iv) H, K and R; (v) N and Q; and (vi) F, Y and W. A conservative mutation or substitution is therefore one that substitutes one amino acid for a member of the same amino acid family (e.g., a substitution of Ser for Thr or Lys for Arg).

By “CpG sites” is meant regions of DNA where a cytosine nucleotide occurs next to a guanine nucleotide in the linear nucleic acid sequence of nucleotides along its length, e.g., -C-phosphate-G-, cytosine and guanine separated by only one phosphate, or a cytosine 5′ to the guanine nucleotide.

As used herein, the terms “dementia with Lewy bodies” and “Lewy body dementia” are used interchangeably to refer a disorder comprising dementia symptoms of fluctuating cognitive impairment, hallucination in which a specific detailed event appears repeatedly, and/or parkinsonism.

As used herein, the terms “effective amount,” “therapeutically effective amount,” and a “sufficient amount” of, an AAV vector described herein refer to a quantity sufficient to, when administered to the subject, including a mammal, for example a human, effect beneficial or desired results, including clinical results. As such, an “effective amount” or synonym thereof depends upon the context in which it is being applied. For example, in the context of treating a neurocognitive or a neuromuscular disorder, it is an amount of the AAV vector sufficient to achieve a treatment response as compared to the response obtained without administration of the AAV vector. The amount of a given AAV vector described herein that will correspond to such an amount will vary depending upon various factors, such as the pharmaceutical formulation, the identity of the subject (e.g., age, sex, weight), and the like, but can nevertheless be routinely determined by one skilled in the art. Also, as used herein, a “therapeutically effective amount” of an AAV vector of the present disclosure is an amount which results in a beneficial or desired result in a subject as compared to a control. As defined herein, a therapeutically effective amount of an AAV vector of the present disclosure may include an amount of from about 1×10⁹ vg/hemisphere to about 9×10¹² vg/hemisphere.

As used herein, the term “endogenous” describes a molecule (e.g., a polypeptide, nucleic acid, or cofactor) that is found naturally in a particular organism (e.g., a human) or in a particular location within an organism (e.g., an organ, a tissue, or a cell, such as a human cell).

As used herein, the terms “frontotemporal dementia” and “FTD” refer to a disorder caused by the degeneration of the brain's frontal lobes and degeneration that may extend to the temporal lobe. FTD is one of the three syndromes caused by frontotemporal lobe degeneration and the second most common cause of early dementia, after AD. Diagnostic criteria according to the Lund-Manchester criteria include onset and gradual progression, early decline in social interpersonal conduct, early impairment in regulation of personal conduct, early emotional blunting, and early loss of insight. Symptoms of FTD may appear between the ages of about 45 to about 65 years (e.g., 50 to about 60) of age. As used herein, “FTD” is intended to include all the stages (such as preclinical stage) and subtypes of the disease.

As used herein, the term “GC content” refers to the quantity of nucleosides in a particular nucleic acid molecule, such as a DNA or RNA polynucleotide, that are either guanosine (G) or cytidine (C) relative to the total quantity of nucleosides present in the nucleic acid molecule. GC content may be expressed as a percentage, for instance, according to the following formula:

GC Content=((Total quantity of guanosine nucleosides)+(Total quantity of cytidine nucleosides)/(Total quantity of nucleosides))×100

As used herein, patients suffering from a “GRN mutation” or “GRN-associated FTD” are those patients that have been diagnosed as having FTD and also contain a deleterious mutation in the GRN gene. Over 70 pathogenic mutations have been reported in the GRN gene, the majority of which result in a premature stop codon and nonsense-mediated decay of truncated GRN mRNA. GRN mutations are described in Gijselinck et al., Hum. Mutat. 29 (12), 1373-1386, (2012) and Pottier et al., J. Neurochem. 138 (Suppl.1), 32:53, (2016), the disclosures of which are incorporated herein by reference as they pertain to human GRN mutations.

As used herein, the term “intron” refers to a region within the coding region of a gene, the nucleotide sequence of which is not translated into the amino acid sequence of the corresponding protein. The term intron also refers to the corresponding region of the RNA transcribed from a gene. In some embodiments, a gene, for example, may contain at minimum two introns, each of which forms the intervening sequence between two exons. Introns are transcribed into pre-mRNA, but are removed during processing, and are not included in the mature mRNA.

An “ITR” is a palindromic nucleic acid, e.g., an inverted terminal repeat, that is about 120 nucleotides to about 250 nucleotides in length and capable of forming a hairpin. The term “ITR” includes the site of the viral genome replication that can be recognized and bound by a parvoviral protein (e.g., Rep78/68). An ITR may be from any adeno-associated virus (AAV), with serotype 2 being preferred. An ITR includes a replication protein binding element (RBE) and a terminal resolution sequence (TRS). The term “ITR” does not require a wild-type parvoviral ITR (e.g., a wild-type nucleic acid sequence may be altered by insertion, deletion, truncation, or missense mutations), as long as the ITR functions to mediate virus packaging, replication, integration, and/or provirus rescue, and the like. The “5′ ITR” is intended to mean the parvoviral ITR located at the 5′ boundary of the nucleic acid molecule; and the term “3′ ITR” is intended to mean the parvoviral ITR located at the 3′ boundary of the nucleic acid molecule.

As used herein, the term “modified nucleotide” refers to a nucleotide or portion thereof (e.g., adenosine, guanosine, thymidine, cytidine, or uridine) that has been altered by one or more enzymatic or synthetic chemical transformations. Exemplary alterations observed in modified nucleotides described herein or known in the art include the introduction of chemical substituents, such as halo, thio, amino, azido, alkyl, acyl, or other functional groups at one or more positions (e.g., the 2′, 3′, and/or 5′ position) of a 2-deoxyribonucleotide or a ribonucleotide.

As used herein, the terms “motor neuron disorder” and “motor neuron disease” refer interchangeably to a group of progressive neurological disorders that destroy motor neurons, the cells that control skeletal muscle activity such as walking, breathing, speaking, and swallowing. Exemplary, non-limiting motor neuron disorders include ALS, progressive bulbar palsy, primary lateral sclerosis, progressive muscular atrophy, spinal muscular atrophy, Kennedy's disease, and post-polio syndrome. It is to be understood that the above list is not all-inclusive.

As used herein, the term “mutation” refers to a change in the nucleotide sequence of a gene (e.g., GRN) or a change in the polypeptide sequence of a protein (e.g., PGRN). Mutations in a gene or protein may occur naturally as a result of, for example, errors in DNA replication, DNA repair, irradiation, and exposure to carcinogens or mutations may be induced as a result of administration of a transgene expressing a mutant gene. Mutations may result from single or multiple nucleotide insertions, deletions, or substitutions.

As used herein, the term “neurocognitive disorder” (NCD) refers to a set of clinical disorders or syndromes in which the primary clinical deficit is cognitive function, such as a deficit in, e.g., complex attention, executive function, learning and memory, language, perceptual-motor function, and social cognition. NCD is characterized as an acquired condition, rather than a developmental one. For example, an NCD is a condition in which disrupted cognition was not evident since birth or very early life, therefore requiring that cognitive function in NCD declined from a previously acquired level. NCD is distinguished from other disorders in which patients present with cognitive impairment in that NCD includes only disorders in which the core deficits are cognitive. NCD may be “major NCD” or “mild NCD.” Major NCD is characterized by significant cognitive decline that interferes with personal independence and normal daily functioning and is not due to delirium or other mental disorder. Mild NCD is characterized by moderate cognitive decline that does not interfere with personal independence and normal daily functioning and is not due to delirium or other mental disorder. Major and mild NCD may also be differentiated on the basis of quantitative cognitive testing across any one of the specific cognitive functions described above. For example, major NCD can be characterized by a score obtained on a cognitive test by a subject identified as having or at risk of developing NCD that is more than two standard deviations away from the mean score of a reference population (e.g., the mean score of a general population) or a score that is in the third percentile of the distribution of scores of the reference population. Mild NCD can be characterized by a score obtained on a cognitive test by a subject identified as having or at risk of developing NCD that is between one to two standard deviations away from the mean score of a reference population or a score that is between the 3^{rd} and 16^{th} percentile of the distribution of scores of the reference population. Non-limiting examples of cognitive tests that can be used to categorize an NCD patient as having either major or mild NCD include AD8, AWV, GPCOG, HRA, MIS, MMSE, MoCA, SLUMS, and Short IQCODE. Furthermore, NCD includes syndrome subtypes that designate the particular etiological origin of the NCD, such as, e.g., FTD, AD, or dementia with Lewy bodies.

As used herein, the terms “neurodegenerative disorder” and “neurodegenerative disease” refer interchangeably to a disorder characterized by progressive loss of the number (e.g., by cell death), structure, and/or function of neurons. In some instances, a neurocognitive or a neuromuscular disorder (e.g., a neurodegenerative disease) may be associated with genetic defects (e.g., a mutation in the GRN gene) protein misfolding, defects in protein degradation, programmed cell death, membrane damage, or other processes. Exemplary, non-limiting neurodegenerative disorders include FTD, AD, PD, dementia with Lewy bodies, ALS, Lou Gehrig's disease, MND, PBP, PMA, PLS, bulbar onset ALS, spinal onset ALS and ALS with multi-system involvement, and a related motor neuron disorder.

As used herein, the term “neuromuscular disorder” refers to a disease impairing the ability of one or more neurons to control the activity of an associated muscle. Examples of neuromuscular disorders are Parkinson's disease (PD), ALS, congenital myasthenic syndrome, congenital myopathy, cramp fasciculation syndrome, Duchenne muscular dystrophy, glycogen storage disease type II, hereditary spastic paraplegia, inclusion body myositis, Isaac's Syndrome, Kearns-Sayre syndrome, Lambert-Eaton myasthenic syndrome, mitochondrial myopathy, muscular dystrophy, myasthenia gravis, myotonic dystrophy, peripheral neuropathy, spinal and bulbar muscular atrophy, spinal muscular atrophy, Stiff person syndrome, Troyer syndrome, and Guillain-Barre syndrome, among others.

It is to be understood that the above lists are not all-inclusive, and that a disorder or disease may fall within various categories. For example, AD can be considered a neurocognitive disorder, and a neurodegenerative disease. Likewise, PD can be considered a neuromuscular disorder and a neurodegenerative disease.

“Nucleic acid” or “polynucleotide,” as used interchangeably herein, refer to polymers of nucleotides of any length and include DNA and RNA.

As used herein, the term “operably linked” refers to a first molecule joined to a second molecule, wherein the molecules are so arranged that the first molecule affects the function of the second molecule. The two molecules may or may not be part of a single contiguous molecule and may or may not be adjacent. For example, a promoter is operably linked to a transcribable polynucleotide molecule if the promoter modulates transcription of the transcribable polynucleotide molecule of interest in a cell. Additionally, two portions of a transcription regulatory element are operably linked to one another if they are joined such that the transcription-activating functionality of one portion is not adversely affected by the presence of the other portion. Two transcription regulatory elements may be operably linked to one another by way of a linker nucleic acid (e.g., an intervening non-coding nucleic acid) or may be operably linked to one another with no intervening nucleotides present.

“Percent (%) sequence identity” with respect to a reference polynucleotide or polypeptide sequence is defined as the percentage of nucleic acids or amino acids in a candidate sequence that are identical to the nucleic acids or amino acids in the reference polynucleotide or polypeptide sequence, after aligning the sequences and introducing gaps, if necessary, to achieve the maximum percent sequence identity. Alignment for purposes of determining percent nucleic acid or amino acid sequence identity can be achieved in various ways that are within the capabilities of one of skill in the art, for example, using publicly available computer software such as BLAST, BLAST-2, or Megalign software. Those skilled in the art can determine appropriate parameters for aligning sequences, including any algorithms needed to achieve maximal alignment over the full length of the sequences being compared. For example, percent sequence identity values may be generated using the sequence comparison computer program BLAST. As an illustration, the percent sequence identity of a given nucleic acid or amino acid sequence, A, to, with, or against a given nucleic acid or amino acid sequence, B, (which can alternatively be phrased as a given nucleic acid or amino acid sequence, A that has a certain percent sequence identity to, with, or against a given nucleic acid or amino acid sequence, B) is calculated as follows:

100 multiplied by (the fraction X/Y)

where X is the number of nucleotides or amino acids scored as identical matches by a sequence alignment program (e.g., BLAST) in that program's alignment of A and B, and where Y is the total number of nucleic acids in B. It will be appreciated that where the length of nucleic acid or amino acid sequence A is not equal to the length of nucleic acid or amino acid sequence B, the percent sequence identity of A to B will not equal the percent sequence identity of B to A.

The terms “polyadenylation signal,” “polyadenylation site,” and “pA” are used interchangeably to herein to mean a nucleic acid sequence sufficient to direct the addition of polyadenosine ribonucleic acid to an RNA molecule expressed in a cell.

As used herein, the term “plasmid” refers to a to an extrachromosomal circular double stranded DNA molecule into which additional DNA segments may be ligated. A plasmid is a type of vector, a nucleic acid molecule capable of transporting another nucleic acid to which it has been linked. Certain plasmids are capable of autonomous replication in a host cell into which they are introduced (e.g., bacterial plasmids having a bacterial origin of replication and episomal mammalian plasmids). Other vectors (e.g., non-episomal mammalian vectors) can be integrated into the genome of a host cell upon introduction into the host cell, and thereby are replicated along with the host genome. Certain plasmids are capable of directing the expression of genes to which they are operably linked.

As used herein, the terms “progranulin” and “PGRN” refer to the secreted trophic factor and precursor peptide for granulin. The gene is located on chromosome 17q21.31 and is known as GRN. The terms “progranulin” and “PGRN” also refer to variants of wild-type progranulin peptides and nucleic acids encoding the same, such as variant proteins having at least 85% sequence identity (e.g., 85%, 86%, 87%, 88%, 89%, 90%, 91%, 92%, 93%, 94%, 95%, 96%, 97%, 98%, 99%, or 99.9% identity, or more) to the amino acid sequence of a wild-type PGRN peptide (e.g., SEQ ID NO. 2) or polynucleotides having at least 85% sequence identity (e.g., 85%, 86%, 87%, 88%, 89%, 90%, 91%, 92%, 93%, 94%, 95%, 96%, 97%, 98%, 99%, or 99.9% identity, or more) to the nucleic acid sequence of a wild-type GRN gene (e.g., SEQ ID NO. 7), provided that the PGRN analog encoded retains the therapeutic function of wild-type PGRN. The terms “progranulin” and “PGRN” may also refer to a PGRN protein in which the natural secretory signal peptide is present. As used herein, “PGRN” refers to the peptide, while “GRN” refers to the gene encoding this protein, as will be appreciated by one of skill in the art.

As used herein, the terms “Parkinson's disease” and “PD” refer to a neurodegenerative disease characterized by motor and non-motor symptoms. Exercise symptoms mainly include dyskinesias, hypotonia, stiffness and progression, where exercise dyskinesia includes exercise-induced relaxation and even anemia. Non-motor symptoms include pain, constipation, delayed gastric emptying, depression, and sleep disorders.

As used herein, the term “promoter” refers to a recognition site on DNA that is bound by an RNA polymerase. The polymerase drives transcription of the transgene. Exemplary promoters suitable for use with the compositions and methods described herein are described, for example, in Sandelin et al., Nat. Rev. Genet. 8:424 (2007), the disclosure of which is incorporated herein by reference as it pertains to nucleic acid regulatory elements.

As used herein, the term “pharmaceutically acceptable” refers to those compounds, materials, compositions and/or dosage forms, which are suitable for contact with the tissues of a subject, such as a mammal (e.g., a human) without excessive toxicity, irritation, allergic response and other problem complications commensurate with a reasonable benefit/risk ratio.

As used herein, the term “secretory signal peptide” refers to a short (usually between 16-60 amino acids) peptide region within the precursor protein that directs secretion of the precursor protein from the cytoplasm of the host into the periplasmic space or into the extracellular space. Such secretory signal peptides are generally located at the amino terminus of the precursor protein. In some embodiments, the secretory signal peptide is linked to the amino terminus. Typically, secretory signal peptides are cleaved during transit through the cellular secretion pathway. Cleavage is not essential as long as the secreted protein retains its desired activity. Exemplary secretory signal peptide includes the PGRN secretory signal peptide.

As used herein, the term “therapeutic protein” refers to (i) a protein whose deficiency or lack of activity is associated with a disorder (e.g., a neurological disorder described herein), as well as (ii) a protein that is not necessarily deficient in a patient, but whose supplementation would nonetheless have a beneficial effect on the patient. Exemplary therapeutic proteins useful in conjunction with the compositions and methods of the disclosure are set forth in Table 5, herein.

As used herein, the term “transfection” refers to any of a wide variety of techniques commonly used for the introduction of exogenous DNA into a prokaryotic or eukaryotic host cell, e.g., electroporation, lipofection, calcium-phosphate precipitation, DEAE-dextran transfection, Nucleofection, squeeze-poration, sonoporation, optical transfection, Magnetofection, impalefection, and the like.

As used herein, the term “transgene” refers to a recombinant nucleic acid (e.g., DNA or cDNA) encoding a gene product (e.g., PGRN). The gene product may be an RNA, peptide, or protein. In addition to the coding region for the gene product, the transgene may include or be operably linked to one or more elements to facilitate or enhance expression, such as a promoter, enhancer(s), destabilizing domain(s), response element(s), reporter element(s), insulator element(s), polyadenylation signal(s) and/or other functional elements. Embodiments of the disclosure may utilize any known suitable promoter, enhancer(s), destabilizing domain(s), response element(s), reporter element(s), insulator element(s), polyadenylation signal(s), and/or other functional elements.

As used herein, the terms “subject” and “patient” refer to an animal (e.g., a mammal, such as a human). A subject to be treated according to the methods described herein may be one who has been diagnosed with FTD or GRN-associated a neurocognitive or a neuromuscular disorder (e.g., a neurodegenerative disorder such as FTD, AD, PD, dementia with Lewy bodies, a related neurocognitive disorder, ALS, or a related motor neuron disorder), or one at risk of developing one or more of these conditions. Diagnosis may be performed by any method or technique known in the art. One skilled in the art will understand that a subject to be treated according to the present disclosure may have been subjected to standard tests or may have been identified, without examination, as one at risk due to the presence of one or more risk factors associated with the disease or condition.

As used herein, the terms “transduction” and “transduce” refer to a method of introducing a viral vector construct or a part thereof into a cell, and subsequent expression of a transgene encoded by the vector construct or part thereof in the cell.

As used herein, “treatment” and “treating” refer to an approach for obtaining beneficial or desired results, e.g., clinical results. Beneficial or desired results can include, but are not limited to, alleviation or amelioration of one or more symptoms or conditions; diminishment of extent of disease or condition; stabilized (i.e., not worsening) state of disease, disorder, or condition; preventing spread of disease or condition; delay or slowing the progress of the disease or condition; amelioration or palliation of the disease or condition; and remission (whether partial or total), whether detectable or undetectable. “Ameliorating” or “palliating” a disease or condition means that the extent and/or undesirable clinical manifestations of the disease, disorder, or condition are lessened and/or time course of the progression is slowed or lengthened, as compared to the extent or time course in the absence of treatment. “Treatment” can also mean prolonging survival as compared to expected survival if not receiving treatment. Those in need of treatment include those already with the condition or disorder, as well as those prone to have the condition or disorder or those in which the condition or disorder is to be prevented.

As used herein, the term “vector” includes a nucleic acid vector, e.g., a DNA vector, such as a plasmid, an RNA vector, virus, or other suitable replicon (e.g., viral vector). A variety of vectors have been developed for the delivery of polynucleotides encoding exogenous proteins into a prokaryotic or eukaryotic cell. Examples of such expression vectors are disclosed in, e.g., WO 1994/011026; incorporated herein by reference as it pertains to vectors suitable for the expression of a gene of interest. Expression vectors suitable for use with the compositions and methods described herein contain a polynucleotide sequence as well as, e.g., additional sequence elements used for the expression of proteins and/or the integration of these polynucleotide sequences into the genome of a mammalian cell. Certain vectors that can be used for the expression of PGRN as described herein include plasmids that contain regulatory sequences, such as promoter and enhancer regions, which direct gene transcription. Other useful vectors for expression of PGRN contain polynucleotide sequences that enhance the rate of translation of these genes or improve the stability or nuclear export of the mRNA that results from gene transcription. These sequence elements include, e.g., 5′ and 3′ untranslated regions, an IRES, and polyadenylation signal site in order to direct efficient transcription of the gene carried on the expression vector. The expression vectors suitable for use with the compositions and methods described herein may also contain a polynucleotide encoding a marker for selection of cells that contain such a vector. Examples of a suitable marker are genes that encode resistance to antibiotics, such as ampicilin, chloramphenicol, kanamycin, nourseothricin, or zeocin.

### DETAILED DESCRIPTION

Described herein are compositions and methods for the treatment of a disorder affecting the central nervous system (CNS) (e.g., a neurocognitive disorder, a neuromuscular disorder, or a neurodegenerative disorder (such as FTD, AD, PD, dementia with Lewy bodies, ALS, or a related neurocognitive or motor neuron disorder) or a lysosomal storage disorder) in a subject (such as a mammalian subject, for example, a human). The compositions and methods described herein are useful for stimulating expression of a therapeutic protein, such as a protein whose deficiency or lack of activity is associated with the disorder of interest or a protein that is not necessarily deficient in a patient, but whose supplementation is likely to have a beneficial effect on the patient. An exemplary therapeutic protein of the disclosure is human progranulin (PGRN) protein, which is particularly useful for treating disorders associated with mutations in the progranulin gene (GRN), such as FTD. The compositions and methods described herein are also useful for stimulating expression of various other therapeutic proteins that may ameliorate, or otherwise benefit patients suffering from, neurocognitive and/or neuromuscular disorders, as well as lysosomal storage disorders. Exemplary therapeutic proteins useful in conjunction with the compositions and methods of the disclosure are described in Table 5, below.

The compositions described herein include an adeno-associated virus (AAV) encoding a therapeutic protein. Therapeutic transgenes that may be used to encode such proteins may include, for example, human GRN or codon-optimized human GRN thereof, which is useful for expression of PGRN protein in a cell. The AAVs described herein may be administered to the patient intrathalamically in an amount of from about 1×10⁹ vg/hemisphere to about 9×10¹² vg/hemisphere. Without being limited by mechanism, the compositions described herein may ameliorate pathology associated with a disorder affecting the central nervous system (e.g., a neurocognitive disorder, a neuromuscular disorder, or a neurodegenerative disorder (such as FTD, AD, PD, dementia with Lewy bodies, ALS, or a related neurocognitive or motor neuron disorder) or a lysosomal storage disorder) by efficaciously stimulating the expression of the therapeutic protein (e.g., PGRN, among others described herein). Using the compositions and methods described herein, one can treat one or more of the above disorders by administering an AAV described herein.

The present invention is based, at least in part, on the discovery that intrathalamic delivery of an AAV including a transgene encoding a therapeutic protein (e.g., PGRN) leads to a surprisingly superior ability to transduce the cortex (e.g., by anterograde and/or retrograde trafficking of the AAV) and induce expression of therapeutic protein (e.g., PGRN) in the cortex. In the context of PGRN gene therapy, this property is particularly beneficial in view of the prevalence of mutations of the GRN gene in mammalian genomes, such as in the genomes of human patients with FTD, which is a disorder characterized by neurodegeneration in the frontal and temporal lobes of the cerebral cortex. The invention is also based, at least in part, upon the identification of an optimum dosing range to achieve said expression of a therapeutic protein (e.g., PGRN) in the cortex following intrathalamic delivery. The optimum dosing range, combined with the method of delivery, result in highly specific transduction and subsequent transgene expression in the CNS relative to peripheral tissues (e.g., liver, lung, and spleen). This specificity is highly desirable, as elevated PGRN expression in peripheral tissues has been associated with certain deleterious effects, including, without limitation, cancer growth and inflammation-associated adverse reactions. A method of delivery that ensures little to no transgene expression in peripheral tissues is, therefore, highly advantageous. Using the compositions and methods described herein, for example, the expression of important, healthy GRN, or codon-optimized variants thereof and their encoded PGRN protein product can be efficaciously enhanced in the cortex.

The sections that follow provide a description of exemplary codon-optimization and methods of production thereof to produce codon-optimized therapeutic transgenes (e.g., human GRN) that may be used in conjunction with the dosing regimens and AAV vectors encoding such constructs described herein to provide methods that may be used to treat a disorder affecting the CNS (e.g., a neurocognitive disorder, a neuromuscular disorder, or a neurodegenerative disorder (such as FTD, AD, PD, dementia with Lewy bodies, ALS, or a related neurocognitive or motor neuron disorder) or a lysosomal storage disorder).

### Neurocognitive and Neuromuscular Disorders

Neurocognitive disorders are defined as a collection of disorders that feature cognitive impairment as a core symptom and that show cognitive decline relative to a previously higher level of cognition (e.g., acquired impairment), rather than a developmental impairment; whilst neuromuscular disorders are featured by progressive muscle weakness. Neurocognitive disorders can be categorized on the basis of their etiological origin. For example, non-limiting examples of neurocognitive disorders may include neurocognitive disorders due to AD, neurocognitive disorders with Lewy bodies (e.g., dementia with Lewy bodies), neurocognitive disorders due to PD, frontotemporal neurocognitive disorders (e.g., FTD), neurocognitive disorders due to a leukodystrophy (e.g., PLOSL), vascular neurocognitive disorders, neurocognitive disorders due to traumatic brain injury, neurocognitive disorders due to HIV infection, substance/medication-induced neurocognitive disorders, neurocognitive disorders due to Huntington's disease, neurocognitive disorders due to prion disease, neurocognitive disorders due to another medical condition, neurocognitive disorders due to multiple etiologies, and unspecified neurocognitive disorders. Non-limiting examples of neuromuscular disorders include PD, ALS, congenital myasthenic syndrome, congenital myopathy, cramp fasciculation syndrome, Duchenne muscular dystrophy, glycogen storage disease type II, hereditary spastic paraplegia, inclusion body myositis, Isaac's Syndrome, Kearns-Sayre syndrome, Lambert-Eaton myasthenic syndrome, mitochondrial myopathy, muscular dystrophy, myasthenia gravis, myotonic dystrophy, peripheral neuropathy, spinal and bulbar muscular atrophy, spinal muscular atrophy, Stiff person syndrome, Troyer syndrome, and Guillain-Barre syndrome, and related motor neuron disorders. The compositions and methods disclosed herein are useful for the treatment of neurocognitive disorders and/or neuromuscular disorders.

Neurocognitive and Neuromuscular Disorders Associated with GRN Mutations

FTD is a clinical syndrome characterized by progressive neurodegeneration in the frontal and temporal lobes of the cerebral cortex. The clinical manifestation of FTD is complex and heterogeneous, but may present as progressive aphasia, decline in cognition (e.g., reduced working memory and executive function), diminished impulse control, emergence of perseverative behaviors, apraxia, apathy, and/or social withdrawal. Neuronal loss in brains of FTD patients is associated with distinct neuropathologies, including mutations in the GRN gene, the presence of tau-positive neuronal and glial inclusions; or ubiquitin (ub)-positive and TAR DNA-binding protein 43 (TDP43)-positive, but tau-negative inclusions. These neuropathologies are considered to be important in the etiology of FTD and also highlight some of the other proteins by which PGRN is thought to interact with, so as to also play a role in other neurodegenerative diseases, such as AD, dementia with Lewy bodies, a related neurocognitive disorder, ALS, and related motor neuron disorders. For example, there is a known relationship between the reduction of PGRN and the accumulation of TDP-43, a protein which has been shown to be a primary component in cytoplasmic aggregates in post-mortem tissue of patients with ALS. Furthermore, nearly half of FTD patients have a first-degree family member with dementia, ALS, or PD, suggesting a strong genetic link to the causes of the diseases, and a number of mutations in chromosome 17q21 have been linked to FTD presentation.

Studies investigating the link between chromosome 17q21 and FTD have found a number of FTD-related mutations in the PGRN gene, GRN. These mutations often result in aggregation and accumulation of ub-positive, TDP43-positive, tau-negative neuropathological inclusions in brains of FTD patients. PGRN is a secreted precursor peptide to a number of mature granulin proteins and is thought to function primarily as a neurotrophic growth factor, promoting neuronal differentiation and survival. PGRN has also been demonstrated to serve anti-inflammatory and neuroprotective functions. PGRN is expressed ubiquitously, but as a result of its association with FTD, significant attention has been directed to the central nervous system (CNS) where it is expressed in multiple cell types including neuronal, glial, and endothelial cells. Over 70 loss-of-function mutations in the GRN gene have been identified in FTD, the vast majority of which result in haploinsufficiency and a reduction in serum PGRN levels by more than a 50%. GRN mutations are described in Gijselinck et al., Hum. Mutat. 29 (12), 1373-86 (2008), the disclosures of which are incorporated herein by reference as they relate to human GRN mutations. Effects of GRN mutations are dose dependent as homozygous patients completely lacking functional PGRN protein develop a lysosomal storage disease known as CLN11 neuronal ceroid lipofuscinosis (NCL), suggesting an additional role for this protein in normal lysosomal function. Neurodegeneration, dementia, and premature cognitive decline are also a hallmark of NCL symptomology.

Clinical management of a neurocognitive or a neuromuscular disorder (e.g., a neurodegenerative disorder such as FTD, AD, PD, dementia with Lewy bodies, a related neurocognitive disorder, ALS, or a related motor neuron disorder) have primarily been employed to ameliorate disease symptomology. For example, in FTD, current approaches to clinical management often employ selective serotonin reuptake inhibitors and antipsychotics to manage the changes in affect and behavior that accompany FTD. This strategy, however, is targeted at ameliorating the symptoms of the disease without addressing its development and progression. Unlike these treatments, the compositions and methods described herein provide the benefit of treating a different biochemical phenomenon that can underlie the development of PGRN-associated pathology. As such, the compositions and methods described herein target the physiological cause of the disease, representing a potential curative therapy. The compositions and methods described herein can be used to treat a neurocognitive or a neuromuscular disorder (e.g., a neurodegenerative disorder such as FTD, AD, PD, dementia with Lewy bodies, a related neurocognitive disorder, ALS, or a related motor neuron disorder) by intrathalamically administering an AAV vector comprising a transgene encoding PGRN. These compositions and methods can be used to treat a neurocognitive or a neuromuscular disorder (e.g., a neurodegenerative disorder such as FTD, AD, PD, dementia with Lewy bodies, a related neurocognitive disorder, ALS, or a related motor neuron disorder) with any etiology, e.g., genetic mutation, environmental toxin, or sporadic. These compositions and methods can also be used to treat patients with GRN-associated FTD. The compositions and methods described herein can be used to treat patients with reduced PGRN activity and/or expression (e.g., a level of expression of endogenous PGRN that is from about 1% to about 40% of the level of endogenous PGRN activity and/or expression observed in a human subject of the same age, gender, and/or body mass index that does not have a neurocognitive or a neuromuscular disorder (e.g., a neurodegenerative disorder such as FTD, AD, PD, dementia with Lewy bodies, a related neurocognitive disorder, ALS, or a related motor neuron disorder) or patients whose GRN mutational status and/or PGRN activity level is unknown. Healthy, physiological levels of 2-10 ng/mL PGRN are observed in the CNS of human subjects who do not have a neurocognitive or a neuromuscular disorder. The compositions and methods described herein may also be administered as a preventative treatment to patients at risk of developing a neurocognitive or a neuromuscular disorder; patients with reduced PGRN activity and/or expression (e.g., a level of expression of endogenous PGRN that is from about 1% to about 40% of the level of endogenous PGRN activity and/or expression observed in a human subject of the same age, gender, and/or body mass index that does not have a neurocognitive or a neuromuscular disorder (e.g., a neurodegenerative disorder such as FTD, AD, PD, dementia with Lewy bodies, a related neurocognitive disorder, ALS, or a related motor neuron disorder); or patients with a mutation in the GRN gene.

According to the methods described herein, a patient can be administered an AAV vector that expresses a transgene encoding the amino acid sequence of SEQ ID NO. 2, below, or a polynucleotide encoding a polypeptide having at least 90% sequence identity (e.g., 90%, 95%, 96%, 97%, 98%, 99%, or more, sequence identity) to the amino acid sequence of SEQ ID NO. 2, or a polynucleotide encoding a polypeptide that contains one or more conservative amino acid substitutions relative to SEQ ID NO. 2 (e.g., 1, 2, 3, 4, 5, 6, 7, 8, 9, or 10 or more conservative amino acid substitutions) e.g., SEQ ID NO: 3, below, provided that the PGRN analog encoded retains the therapeutic function of wild-type PGRN. In some embodiments, the polynucleotide encoding wild-type PGRN may be a codon-optimized polynucleotide, as described in detail below.

Wild-type human PGRN (Gen Bank accession number: NP_002078.1) has the amino acid sequence of:

(SEQ ID NO. 2)
MWTLVSWVALTAGLVAGTRCPDGQFCPVACCLDPGGASYSCCRPLLDKW
PTTLSRHLGGPCQVDAHCSAGHSCIFTVSGTSSCCPFPEAVACGDGHHC
CPRGFHCSADGRSCFQRSGNNSVGAIQCPDSQFECPDFSTCCVMVDGSW
GCCPMPQASCCEDRVHCCPHGAFCDLVHTRCITPTGTHPLAKKLPAQRT
NRAVALSSSVMCPDARSRCPDGSTCCELPSGKYGCCPMPNATCCSDHLH
CCPQDTVCDLIQSKCLSKENATTDLLTKLPAHTVGDVKCDMEVSCPDGY
TCCRLQSGAWGCCPFTQAVCCEDHIHCCPAGFTCDTQKGTCEQGPHQVP
WMEKAPAHLSLPDPQALKRDVPCDNVSSCPSSDTCCQLTSGEWGCCPIP
EAVCCSDHQHCCPQGYTCVAEGQCQRGSEIVAGLEKMPARRASLSHPRD
IGCDQHTSCPVGQTCCPSLGGSWACCQLPHAVCCEDRQHCCPAGYTCNV
KARSCEKEVVSAQPATFLARSPHVGVKDVECGEGHFCHDNQTCCRDNRQ
GWACCPYRQGVCCADRRHCCPAGFRCAARGTKCLRREAPRWDAPLRDPA
LRQLL

Wild-type human GRN (GenBank accession number: NM_002087.3) has the nucleic acid sequence of:

(SEQ ID NO. 10)
ATTCTCCAATCACATGATCCCTAGAAATGGGGTGTGGGGCGAGAGGAAG
CAGGGAGGAGAGTGATTTGAGTAGAAAAGAAACACAGCATTCCAGGCTG
GCCCCACCTCTATATTGATAAGTAGCCAATGGGAGCGGGTAGCCCTGAT
CCCTGGCCAATGGAAACTGAGGTAGGCGGGTCATCGCGCTGGGGTCTGT
AGTCTGAGCGCTACCCGGTTGCTGCTGCCCAAGGACCGCGGAGTCGGAC
GCAGGCAGACCATGTGGACCCTGGTGAGCTGGGTGGCCTTAACAGCAGG
GCTGGTGGCTGGAACGCGGTGCCCAGATGGTCAGTTCTGCCCTGTGGCC
TGCTGCCTGGACCCCGGAGGAGCCAGCTACAGCTGCTGCCGTCCCCTTC
TGGACAAATGGCCCACAACACTGAGCAGGCATCTGGGTGGCCCCTGCCA
GGTTGATGCCCACTGCTCTGCCGGCCACTCCTGCATCTTTACCGTCTCA
GGGACTTCCAGTTGCTGCCCCTTCCCAGAGGCCGTGGCATGCGGGGATG
GCCATCACTGCTGCCCACGGGGCTTCCACTGCAGTGCAGACGGGCGATC
CTGCTTCCAAAGATCAGGTAACAACTCCGTGGGTGCCATCCAGTGCCCT
GATAGTCAGTTCGAATGCCCGGACTTCTCCACGTGCTGTGTTATGGTCG
ATGGCTCCTGGGGGTGCTGCCCCATGCCCCAGGCTTCCTGCTGTGAAGA
CAGGGTGCACTGCTGTCCGCACGGTGCCTTCTGCGACCTGGTTCACACC
CGCTGCATCACACCCACGGGCACCCACCCCCTGGCAAAGAAGCTCCCTG
CCCAGAGGACTAACAGGGCAGTGGCCTTGTCCAGCTCGGTCATGTGTCC
GGACGCACGGTCCCGGTGCCCTGATGGTTCTACCTGCTGTGAGCTGCCC
AGTGGGAAGTATGGCTGCTGCCCAATGCCCAACGCCACCTGCTGCTCCG
ATCACCTGCACTGCTGCCCCCAAGACACTGTGTGTGACCTGATCCAGAG
TAAGTGCCTCTCCAAGGAGAACGCTACCACGGACCTCCTCACTAAGCTG
CCTGCGCACACAGTGGGGGATGTGAAATGTGACATGGAGGTGAGCTGCC
CAGATGGCTATACCTGCTGCCGTCTACAGTCGGGGGCCTGGGGCTGCTG
CCCTTTTACCCAGGCTGTGTGCTGTGAGGACCACATACACTGCTGTCCC
GCGGGGTTTACGTGTGACACGCAGAAGGGTACCTGTGAACAGGGGCCCC
ACCAGGTGCCCTGGATGGAGAAGGCCCCAGCTCACCTCAGCCTGCCAGA
CCCACAAGCCTTGAAGAGAGATGTCCCCTGTGATAATGTCAGCAGCTGT
CCCTCCTCCGATACCTGCTGCCAACTCACGTCTGGGGAGTGGGGCTGCT
GTCCAATCCCAGAGGCTGTCTGCTGCTCGGACCACCAGCACTGCTGCCC
CCAGGGCTACACGTGTGTAGCTGAGGGGCAGTGTCAGCGAGGAAGCGAG
ATCGTGGCTGGACTGGAGAAGATGCCTGCCCGCCGGGCTTCCTTATCCC
ACCCCAGAGACATCGGCTGTGACCAGCACACCAGCTGCCCGGTGGGGCA
GACCTGCTGCCCGAGCCTGGGTGGGAGCTGGGCCTGCTGCCAGTTGCCC
CATGCTGTGTGCTGCGAGGATCGCCAGCACTGCTGCCCGGCTGGCTACA
CCTGCAACGTGAAGGCTCGATCCTGCGAGAAGGAAGTGGTCTCTGCCCA
GCCTGCCACCTTCCTGGCCCGTAGCCCTCACGTGGGTGTGAAGGACGTG
GAGTGTGGGGAAGGACACTTCTGCCATGATAACCAGACCTGCTGCCGAG
ACAACCGACAGGGCTGGGCCTGCTGTCCCTACCGCCAGGGCGTCTGTTG
TGCTGATCGGCGCCACTGCTGTCCTGCTGGCTTCCGCTGCGCAGCCAGG
GGTACCAAGTGTTTGCGCAGGGAGGCCCCGCGCTGGGACGCCCCTTTGA
GGGACCCAGCCTTGAGACAGCTGCTGTGAGGGACAGTACTGAAGACTCT
GCAGCCCTCGGGACCCCACTCGGAGGGTGCCCTCTGCTCAGGCCTCCCT
AGCACCTCCCCCTAACCAAATTCTCCCTGGACCCCATTCTGAGCTCCCC
ATCACCATGGGAGGTGGGGCCTCAATCTAAGGCCTTCCCTGTCAGAAGG
GGGTTGTGGCAAAAGCCACATTACAAGCTGCCATCCCCTCCCCGTTTCA
GTGGACCCTGTGGCCAGGTGCTTTTCCCTATCCACAGGGGTGTTTGTGT
GTGTGCGCGTGTGCGTTTCAATAAAGTTTGTACACTTTCTTAAAAAAAA
AAAA

### Methods of Treatment

The present disclosure is based, at least in part, on the discovery that AAV vectors encoding PGRN that are delivered to a patient by way of the routes of administration described herein (e.g., intrathalamically) and in the dosing quantities described herein are capable of effectuating PGRN expression levels in the CNS of a patient on the order of double-digit ng/ml (see, e.g., the working examples described below). This discovery is important, as physiologic levels of PGRN protein in the CNS of a healthy human subject are on the order of 2-6 ng/ml (measured in the patient's cerebrospinal fluid). Accordingly, the PGRN-encoding compositions described herein are capable of engendering physiologic PGRN expression levels in the CNS of a human subject. Guided by this discovery, the inventors have found that other therapeutic proteins can be delivered in physiologically relevant amounts using the compositions and methods described herein, as a wide variety of other therapeutic proteins also have healthy concentration levels on the order of up to double-digit ng/ml in the CNS. The following table provides a list of exemplary therapeutic proteins of the disclosure and their corresponding physiologic expression levels in the CNS.

TABLE 2

| | | | | Location at | |
|---|---|---|---|---|---|
| | Associated | Physiologic | | which Level | |
| Therapeutic Protein | Disease | Concentration | | is Measured | Citation |
| Arylsulfatase A | Metachromatic | 100 ng/mg | | Brain | Colle et al., |
| Enzyme (ARSA) | leukodystrophy | protein | | | Human |
| | (MLD) | | | | Molecular |
| | | | | | Genetics |
| | | | | | 19: 147-158 |
| | | | | | (2009) |
| Glial-derived | Alzheimer's | <10 | ng/mL | Brain | Kells et al., |
| Neurotrophic Factor | disease, lysosomal | | | | Proc. Natl. |
| (GDNF) | storage disorders, | | | | Acad. Sci. |
| | and other | | | | USA |
| | disorders with a | | | | 106: 2407- |
| | strong cortical | | | | 2411 (2009) |
| | manifestation | | | | |
| Hexosaminidase- | GM2 | HexA: 20 | | Plasma | Golebiowski |
| alpha and | gangliosidoses | ng/mL | | | et al., |
| hexosaminidase-beta | (e.g., Sandhoff | HexB: 40 | | | Human |
| | disease and Tay- | ng/mL | | | Gene |
| | Sachs disease) | | | | Therapy |
| | | | | | 28: 510-522 |
| | | | | | (2017) |
| Brain-derived | Alzheimer's | up to 50 pg/mL | | CSF | Grundström |
| neurotrophic factor | disease, | | | | et al., |
| (BDNF) | Parkinson's, | | | | Neuroreport |
| | Huntington's | | | | 11: 1781- |
| | | | | | 1783 (2000) |
| BDNF | Alzheimer's | 25 | pg/mL | prefrontal | Issa et al., |
| | disease | | | cortex | Neurobiol |
| | Parkinson's, | | | | Dis. 39: 327- |
| | Huntington's | | | | 333 (2010) |
| Apolipoprotein E | Alzheimer's | 4.5 | ng/mL | CSF | Hesse et al., |
| (ApoE) | Disease | | | | Neurochem |
| | | | | | Res. 25: 511- |
| | | | | | 517 (2000) |
| ApoE | Alzheimer's | 150 ng/mg | | Cortex | Beffert et al., |
| | Disease | protein | | | Brain Res. |
| | | | | | 843: 87-94 |
| | | | | | (1999) |
| Glucocerebrosidase | Gaucher Disease, | 0.02 ng/ml- | | CSF | Abeliovich et |
| | Parkinson's | 0.14 ng/ml | | | al., J. |
| | Disease | | | | Parkinsons |
| | | | | | Dis. S183- |
| | | | | | S188 (2021) |

Exemplary subjects that may be treated as described herein are subjects having or at risk of developing a neurocognitive or a neuromuscular disorder (e.g., a neurodegenerative disorder), such as FTD AD, PD, dementia with Lewy bodies, a related neurocognitive disorder, ALS, or a related motor neuron disorder. The type of FTD may be GRN-associated FTD, sporadic FTD, FTD caused by an environmental toxin, e.g., herbicides or pesticides, or FTD associated with a non-GRN mutation, e.g., a mutation in one or more of the genes associated with FTD. The compositions and methods described herein can be used to treat patients with reduced PGRN activity and/or expression (e.g., a level of expression of endogenous PGRN that is from about 1% to about 40% of the level of endogenous PGRN expression observed in a human subject of the same age, gender, and/or body mass index that does not have a neurocognitive or a neuromuscular disorder (e.g., a neurodegenerative disorder such as FTD, AD, PD, dementia with Lewy bodies, a related neurocognitive disorder, ALS, or a related motor neuron disorder) or patients whose GRN mutational status and/or PGRN activity level is unknown. The compositions and methods described herein may also be administered as a preventative treatment to patients at risk of developing a neurocognitive or a neuromuscular disorder (e.g., a neurodegenerative disorder such as FTD, AD, PD, dementia with Lewy bodies, a related neurocognitive disorder, ALS, or a related motor neuron disorder) e.g., patients with a GRN mutation, patients with reduced PGRN activity and/or expression (e.g., a level of expression of endogenous PGRN that is from about 1% to about 40% of the level of endogenous PGRN expression observed in a human subject of the same age, gender, and/or body mass index that does not have a neurocognitive or a neuromuscular disorder (e.g., a neurodegenerative disorder such as FTD, AD, PD, dementia with Lewy bodies, a related neurocognitive disorder, ALS, or a related motor neuron disorder); patients with a mutation in one or more of the genes associated with a neurocognitive or a neuromuscular disorder (e.g., a neurodegenerative disorder such as FTD, AD, PD, dementia with Lewy bodies, a related neurocognitive disorder, ALS, or a related motor neuron disorder); or patients exposed to an environmental toxin associated with a neurocognitive or a neuromuscular disorder (e.g., a neurodegenerative disorder such as FTD, AD, PD, dementia with Lewy bodies, a related neurocognitive disorder, ALS, or a related motor neuron disorder). Patients at risk for a neurocognitive or a neuromuscular disorder (e.g., a neurodegenerative disorder such as FTD, AD, PD, dementia with Lewy bodies, a related neurocognitive disorder, ALS, or a related motor neuron disorder) may show early symptoms of a neurocognitive or a neuromuscular disorder (e.g., a neurodegenerative disorder such as FTD, AD, PD, dementia with Lewy bodies, a related neurocognitive disorder, ALS, or a related motor neuron disorder) or may not yet be symptomatic when treatment is administered.

In some embodiments, the methods and compositions described herein may be administered to patients with GRN mutations that include, for example, frameshift mutations (e.g., p.C31LfsX35, p.C31LfsX35, p.S82VfsX174, p.L271LfsX174, and/or p.T382NfsX32 mutations), missense mutations (p.C521Y, p.A9D, p.P248L, p.R432C, p.C139R, p.C521Y, and/or p.C139R mutations), nonsense mutations (e.g., p.Q125X mutation), insertion mutations (e.g., c.1145insA mutation), and/or transversion mutation (e.g., p.0 (IVS1+5G>C mutation). In some embodiments, the methods and compositions described herein may be administered to patients carrying any other pathogenic mutation in the GRN gene. For example, pathogenic mutations in the GRN gene may be any of the mutations discussed in Gijselinck et al., Human Mutation 29 (12), 1373-1386, (2012), the disclosure of which is incorporated herein by reference as it pertains to human GRN mutations.

In some embodiments, the disclosure provides a method of treating a neurocognitive or a neuromuscular disorder (e.g., a neurodegenerative disorder such as FTD, AD, PD, dementia with Lewy bodies, a related neurocognitive disorder, ALS, or a related motor neuron disorder) in a human patient in need thereof.

In some embodiments, the disclosure provides a method of improving cognitive function in a human patient diagnosed as having a neurocognitive or a neuromuscular disorder (e.g., a neurodegenerative disorder such as FTD, AD, PD, dementia with Lewy bodies, a related neurocognitive disorder, ALS, or a related motor neuron disorder).

In some embodiments, the disclosure provides a method of expressing, or restoring expression of, PGRN in the brain (e.g., frontal cortex) of a human patient diagnosed as having a neurocognitive or a neuromuscular disorder (e.g., a neurodegenerative disorder such as FTD, AD, PD, dementia with Lewy bodies, a related neurocognitive disorder, ALS, or a related motor neuron disorder).

### Polynucleotides Encoding PGRN

PGRN activity is reduced in patients with FTD. The compositions and methods described herein target this dysfunction by administering an AAV vector expressing a transgene encoding PGRN. Such a construct can be produced using methods well known to those of skill in the field.

Recognition and binding of the polynucleotide encoding PGRN by mammalian RNA polymerase is important for gene expression. As such, one may include sequence elements within the polynucleotide that exhibit a high affinity for transcription factors that recruit RNA polymerase and promote the assembly of the transcription complex at the transcription initiation site. Such sequence elements include, e.g., a mammalian promoter, the sequence of which can be recognized and bound by specific transcription initiation factors and ultimately RNA polymerase. Examples of mammalian promoters have been described in Smith et al., Mol. Sys. Biol., 3:73, online publication, the disclosure of which is incorporated herein by reference.

Polynucleotides suitable for use with the compositions and methods described herein also include those that encode PGRN downstream of a mammalian promoter. Promoters that are useful for the expression of GRN in mammalian cells include, e.g., synapsin promoter, a tetracycline-controlled transactivator protein (tTA) promoter, a cytomegalovirus (CMV) promoter, a reverse tetracycline-controlled transactivator protein (rTA) promoter, a U1 promoter, a U6 promoter, a U7 promoter, a prion promoter, a phosphoglycerate kinase (PGK) promoter, a CB7 promoter, an H1 promoter, a CMV-chicken β-actin (CBA) promoter, a glial fibrillary acidic protein (GFAP) promoter, a calcium/calmodulin-dependent protein kinase Ill promoter, a tubulin alpha I promoter, a microtubulin-associated protein IB (MAP IB) promoter, a neuron-specific enolase promoter, a platelet-derived growth factor beta chain promoter, a neurofilament light chain promoter, a neuron-specific VGF gene promoter, a neuronal nuclei (NeuN) promoter, a adenomatous polyposis coli (APC) promoter, an ionized calcium-binding adapter molecule 1 (Iba-1) promoter, or a homeobox protein 9 (HB9) promoter, elongation factor 1-alpha (EF1a) promoter, CD68 molecule (CD68) promoter (see Dahl et al., Mol. Ther. 23:835 (2015), incorporated herein by reference as it pertains to the use of PGK and CD68 promoters to express GRN), C-X3-C motif chemokine receptor 1 (CX3CR1) promoter, integrin subunit alpha M (ITGAM) promoter, allograft inflammatory factor 1 (AIF1) promoter, purinergic receptor P2Y12 (P2Y12) promoter, transmembrane protein 119 (TMEM119) promoter, and colony stimulating factor 1 receptor (CSF1R) promoter. In some embodiments, the promoter is a synapsin promoter.

In some embodiments, the transgene encoding PGRN is operably linked to a promoter that is active in a neuronal cell (e.g., synapsin) and/or a glial cell.

Other DNA sequence elements that may be included in polynucleotides for use in the compositions and methods described herein are enhancer sequences. Enhancers represent another class of regulatory elements that induce a conformational change in the polynucleotide containing the gene of interest such that the DNA adopts a three-dimensional orientation that is favorable for binding of transcription factors and RNA polymerase at the transcription initiation site. Thus, polynucleotides for use in the compositions and methods described herein include those that encode PGRN and additionally include a mammalian enhancer sequence. Many enhancer sequences are now known from mammalian genes, and examples are enhancers from the genes that encode mammalian globin, elastase, albumin, a-fetoprotein, and insulin. Enhancers for use in the compositions and methods described herein also include those that are derived from the genetic material of a virus capable of infecting a eukaryotic cell. Examples are the SV40 enhancer on the late side of the replication origin (bp 100-270), the CMV early promoter enhancer, the polyoma enhancer on the late side of the replication origin, and adenovirus enhancers. Additional enhancer sequences that induce activation of eukaryotic gene transcription are disclosed in Yaniv et al., Nature 297:17 (1982).

### Exemplary PGRN

In one approach, the invention provides a PGRN that has an amino acid sequence that is at least 85% (e.g., 85%, 86%, 87%, 88%, 89%, 90%, 91%, 92%, 93%, 94%, 95%, 96%, 97%, 98%, or 99%) identical to the amino acid sequence of SEQ ID NO: 2. For example, in some embodiments, the PGRN has an amino acid sequence that is at least 86% identical to the amino acid sequence of SEQ ID NO: 2. In some embodiments, the PGRN has an amino acid sequence that is at least 87% identical to the amino acid sequence of SEQ ID NO: 2. In some embodiments, the PGRN has an amino acid sequence that is at least 88% identical to the amino acid sequence of SEQ ID NO: 2. In some embodiments, the PGRN has an amino acid sequence that is at least 89% identical to the amino acid sequence of SEQ ID NO: 2. In some embodiments, the PGRN has an amino acid sequence that is at least 90% identical to the amino acid sequence of SEQ ID NO: 2. In some embodiments, THE PGRN has an amino acid sequence that is at least 91% identical to the amino acid sequence of SEQ ID NO: 2. In some embodiments, the PGRN has an amino acid sequence that is at least 92% identical to the amino acid sequence of SEQ ID NO: 2. In some embodiments, the PGRN has an amino acid sequence that is at least 93% identical to the amino acid sequence of SEQ ID NO: 2. In some embodiments, the PGRN has an amino acid sequence that is at least 94% identical to the amino acid sequence of SEQ ID NO: 2. In some embodiments, the PGRN has an amino acid sequence that is at least 95% identical to the amino acid sequence of SEQ ID NO: 2. In some embodiments, the PGRN has an amino acid sequence that is at least 96% identical to the amino acid sequence of SEQ ID NO: 2. In some embodiments, the PGRN has an amino acid sequence that is at least 97% identical to the amino acid sequence of SEQ ID NO: 2. In some embodiments, the PGRN has an amino acid sequence that is at least 98% identical to the amino acid sequence of SEQ ID NO: 2. In some embodiments, the PGRN has an amino acid sequence that is at least 99% identical to the amino acid sequence of SEQ ID NO: 2. In some embodiments, the PGRN encodes a protein that is identical to the amino acid sequence of SEQ ID NO: 2.

### Secretory Signal Peptides

Polynucleotides encoding PGRN may include one or more polynucleotides encoding a secretory signal peptide. Secretory signal peptides may have amino acid sequences of 5-30 residues in length, and may be located upstream of (i.e., 5′ to) a polynucleotide encoding PGRN. These secretory signal peptides allow for the recognition of the nascent polypeptides during synthesis by signal recognition particles resulting in translocation to the ER, packaging into transport vesicles, and finally, secretion. Exemplary secretory signal peptides for protein secretion are those from PGRN, IGF-II, alpha-1 antitrypsin, IL-2, IL-6, CD5, immunoglobulins, trypsinogen, serum albumin, prolactin, elastin, tissue plasminogen activator signal peptide (tPA-SP), and insulin. In some embodiments, pluripotent cells (e.g., ESCs, iPSCs, or CD34+ cells) expressing a secreted form of PGRN may be utilized as a therapeutic strategy to correct a protein deficiency (e.g., PGRN) by infusing the missing protein into the bloodstream.-As the blood perfuses patient tissues, PGRN is taken up by cells and transported to its site of action.

### Codon Optimization

The compositions and methods described herein can be used to optimize the nucleic acid sequence of GRN or RNA equivalent thereof encoding PGRN so as to achieve, for instance, enhanced expression of PGRN in a particular cell type. For example, using the compositions and methods described herein, genes and RNA equivalents thereof can be optimized for tissue-specific expression of an encoded protein, such as PGRN. Genes and RNA equivalents thereof optimized using the compositions and methods described herein can be synthesized by chemical synthesis techniques and may be amplified, for instance, using polymerase chain reaction (PCR)-based amplification methods or by transfection of the gene into a cell, such as a bacterial cell or mammalian cell capable of replicating exogenous nucleic acids.

The genes and RNA equivalents described herein can have important clinical utility. For example, FTD is a manifestation of a deficiency in the native PGRN protein. With the advent of gene therapy, a wide array of vectors and gene delivery techniques have been developed for the introduction of exogenous protein-coding nucleic acids into target cells (e.g., human cells). However, there remains a need for a unified set of guidelines one can follow in order to optimize the sequence of an exogenous transgene encoding PGRN so as to achieve robust and stable expression of the protein in the cell of interest.

Single-nucleotide mutations that preserve the amino acid sequence of the encoded protein can be informed, for instance, by the standard genetic code, represented in Table 3, below, compiled by the National Center for Biotechnology Information, Bethesda, Maryland, USA.

TABLE 3
Standard genetic code

| | Amino | | Amino | | Amino | | Amino |
|---|---|---|---|---|---|---|---|
| Codon | Acid | Codon | Acid | Codon | Acid | Codon | Acid |
| GCG | Ala | CAT | His | CCG | Pro | TCT | Ser |
| GCA | Ala | CAC | His | CCA | Pro | TCC | Ser |
| GCT | Ala | ATA | Ile | CCT | Pro | ACG | Thr |
| GCC | Ala | ATT | Ile | CCC | Pro | ACA | Thr |
| TGT | Cys | ATC | Ile | CAG | Gln | ACT | Thr |
| TGC | Cys | AAG | Lys | CAA | Gln | ACC | Thr |
| GAT | Asp | AAA | Lys | AGG | Arg | GTG | Val |
| GAC | Asp | TTG | Leu | AGA | Arg | GTA | Val |
| GAG | Glu | TTA | Leu | CGG | Arg | GTT | Val |
| GAA | Glu | CTG | Leu | CGA | Arg | GTC | Val |
| TTT | Phe | CTA | Leu | CGT | Arg | TGG | Trp |
| TTC | Phe | CTT | Leu | CGC | Arg | TAT | Tyr |
| GGG | Gly | CTC | Leu | AGT | Ser | TAC | Tyr |
| GGA | Gly | ATG | Met | AGC | Ser | TGA | Stop |
| GGT | Gly | AAT | Asn | TCG | Ser | TAG | Stop |
| GGC | Gly | AAC | Asn | TCA | Ser | TAA | Stop |

The codon-optimization process can be performed iteratively. For instance, one of skill in the art can begin with a wild-type gene sequence (e.g., excluding intronic DNA) and introduce substitutions into this sequence that reduce the sequence identity of the gene relative to the genes that are expressed within a target cell (e.g., genes whose expression levels are among the top 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, or more, of gene expression levels in the intended target cell). This process can be repeated until all codons within the gene of interest have been evaluated for the opportunity to introduce single-nucleotide substitutions that can reduce sequence identity relative to the genes expressed at high levels within the target cell. Alternatively, one can begin with a gene sequence that has previously been modified relative to the wild-type sequence of the gene, for instance, by incorporating codon substitutions that increase the GC content of the gene and/or that reduce CpG content of the gene relative to the wild-type sequence. The sequence of the resulting gene can subsequently be aligned to the coding strands of the genes expressed in a desired target cell (e.g., genes whose expression levels are among the top 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, or more, of gene expression levels in the intended target cell), and iterative codon substitutions can be introduced throughout the gene in order to minimize the sequence identity of the previously-modified gene with respect to the genes expressed at high levels within the target cell.

### Preparation of Codon-Optimized Genes

Once designed, the final codon-optimized gene can be prepared, for instance, by solid phase nucleic acid procedures known in the art. For instance, to perform the chemical synthesis of nucleic acid molecules, such as DNA, RNA and the like, a solid phase synthesis process using a phosphoramidite method can be employed. According to this procedure, a nucleic acid is generally synthesized by the following steps.

First, a 5-OH-protected nucleoside that will occur at the 3′ terminal end of the nucleic acid to be synthesized is esterified via the 3′-OH function to a solid support by appending the nucleoside to a cleavable linker. Then, the support for solid phase synthesis on which the nucleoside is immobilized can be placed in a reaction column which is then set on an automated nucleic acid synthesizer.

Thereafter, an iterative synthetic process including the following steps can be performed in the reaction column according to a synthesis program of the automated nucleic acid synthesizer:
(1) a step of deprotection of the 5′-OH moiety of the protected, immobilized nucleoside (e.g., with an acid such as trichloroacetic acid in dichloromethane solution or the like to remove acid-labile hydroxyl protecting groups);
(2) a step of coupling a 5-OH-protected nucleosidephosphoramidite with the deprotected 5′-OH group of the immobilized nucleoside in the presence of an activator (e.g., tetrazole or the like);
(3) a step of capping the unreacted 5′—OH group of the 3′-terminal nucleoside (e.g., with acetic anhydride or the like); and
(4) a step of oxidizing the immobilized phosphite substituent (e.g., with aqueous iodine or the like).

The above process can be repeated to elongate the nucleic acid as needed in a 3′-to-5′ direction. 5′ terminal direction is promoted, and a nucleic acid having a desired sequence is synthesized.

Lastly, the cleavable linker is hydrolyzed (e.g., with aqueous ammonia, methylamine solution, or the like) to cleave the synthesized nucleic acid from the solid phase support. Procedures such as the foregoing for the chemical synthesis of nucleic acids are known in the art and are described, for instance, in U.S. Pat. No. 8,835,656, the disclosure of which is incorporated herein by reference as it pertains to protocols for the synthesis of nucleic acid molecules.

Additionally, the prepared gene can be amplified, for instance, using PCR-based techniques described herein or known in the art, and/or by transformation of DH5a E. coli with a plasmid containing the designed gene. The bacteria can subsequently be cultured so as to amplify the DNA therein, and the gene can be isolated plasmid purification techniques known in the art, followed optionally by a restriction digest and/or sequencing of the plasmid to verify the identity codon-optimized gene.

### Exemplary Codon-Optimized PGRN

In one approach, the invention provides a codon-optimized PGRN that has a nucleic acid sequence that is at least 85% (e.g., 85%, 86%, 87%, 88%, 89%, 90%, 91%, 92%, 93%, 94%, 95%, 96%, 97%, 98%, or 99%) identical to the nucleic acid sequence of SEQ ID NO: 3. For example, in some embodiments, the PGRN has a nucleic acid sequence that is at least 86% identical to the nucleic acid sequence of SEQ ID NO: 3. In some embodiments, the PGRN has a nucleic acid sequence that is at least 87% identical to the nucleic acid sequence of SEQ ID NO: 3. In some embodiments, the PGRN has a nucleic acid sequence that is at least 88% identical to the nucleic acid sequence of SEQ ID NO: 3. In some embodiments, the PGRN has a nucleic acid sequence that is at least 89% identical to the nucleic acid sequence of SEQ ID NO: 3. In some embodiments, the PGRN has a nucleic acid sequence that is at least 90% identical to the nucleic acid sequence of SEQ ID NO: 3. In some embodiments, THE PGRN has a nucleic acid sequence that is at least 91% identical to the nucleic acid sequence of SEQ ID NO: 3. In some embodiments, the PGRN has a nucleic acid sequence that is at least 92% identical to the nucleic acid sequence of SEQ ID NO: 3. In some embodiments, the PGRN has a nucleic acid sequence that is at least 93% identical to the nucleic acid sequence of SEQ ID NO: 3. In some embodiments, the PGRN has a nucleic acid sequence that is at least 94% identical to the nucleic acid sequence of SEQ ID NO: 3. In some embodiments, the PGRN has a nucleic acid sequence that is at least 95% identical to the nucleic acid sequence of SEQ ID NO: 3. In some embodiments, the PGRN has a nucleic acid sequence that is at least 96% identical to the nucleic acid sequence of SEQ ID NO: 3. In some embodiments, the PGRN has a nucleic acid sequence that is at least 97% identical to the nucleic acid sequence of SEQ ID NO: 3. In some embodiments, the PGRN has a nucleic acid sequence that is at least 98% identical to the nucleic acid sequence of SEQ ID NO: 3. In some embodiments, the PGRN has a nucleic acid sequence that is at least 99% identical to the nucleic acid sequence of SEQ ID NO: 3. In some embodiments, the PGRN encodes a protein that is identical to the nucleic acid sequence of SEQ ID NO: 3.

### AAVs for Delivery of PGRN to Target Cells

Viral genomes provide a rich source of vectors that can be used for the efficient delivery of a gene of interest into the genome of a target cell (e.g., a mammalian cell, such as a human cell). Viral genomes are particularly useful vectors for gene delivery because the polynucleotides contained within such genomes are typically incorporated into the genome of a target cell by generalized or specialized transduction. These processes occur as part of the natural viral replication cycle, and do not require added proteins or reagents in order to induce gene integration. Examples of viral vectors include AAV.

Nucleic acids of the compositions and methods described herein may be incorporated into recombinant AAV (rAAV) vectors and/or virions in order to facilitate their introduction into a cell. AAV vectors can be used in the central nervous system, and appropriate promoters and serotypes are discussed in Pignataro et al., J Neural Transm (2017), epub ahead of print, the disclosure of which is incorporated herein by reference as it pertains to promoters and AAV serotypes useful in CNS gene therapy. rAAV vectors useful in the compositions and methods described herein are recombinant nucleic acid constructs that include (1) a heterologous sequence to be expressed (e.g., a polynucleotide encoding PGRN) and (2) viral sequences that facilitate integration and expression of the heterologous genes. The viral sequences may include those sequences of AAV that are required in cis for replication and packaging (e.g., functional ITRs) of the DNA into a virion. Such rAAV vectors may also contain marker or reporter genes. Useful rAAV vectors have one or more of the AAV WT genes deleted in whole or in part but retain functional flanking ITR sequences. The AAV ITRs may be of any serotype suitable for a particular application. Methods for using rAAV vectors are described, for example, in Tai et al., J. Biomed. Sci. 7:279 (2000), and Monahan and Samulski, Gene Delivery 7:24 (2000), the disclosures of each of which are incorporated herein by reference as they pertain to AAV vectors for gene delivery.

The nucleic acids and vectors described herein can be incorporated into a rAAV virion in order to facilitate introduction of the nucleic acid or vector into a cell. The capsid proteins of AAV compose the exterior, non-nucleic acid portion of the virion and are encoded by the AAV cap gene. The cap gene encodes three viral coat proteins, VP1, VP2, and VP3, which are required for virion assembly. The construction of rAAV virions has been described, for example, in U.S. Pat. Nos. 5,173,414; 5,139,941; 5,863,541; 5,869,305; 6,057,152; and 6,376,237; as well as in Rabinowitz et al., J. Virol. 76:791 (2002) and Bowles et al., J. Virol. 77:423 (2003), the disclosures of each of which are incorporated herein by reference as they pertain to AAV vectors for gene delivery.

rAAV virions useful in conjunction with the compositions and methods described herein include those derived from a variety of AAV serotypes including AAV 1, 2, 3, 4, 5, 6, 7, 8, 9, 10 and rh74. For targeting cells located in or delivered to the central nervous system, AAV2, AAV9, and AAV10 may be particularly useful. Construction and use of AAV vectors and AAV proteins of different serotypes are described, for example, in Chao et al., Mol. Ther. 2:619 (2000); Davidson et al., Proc. Natl. Acad. Sci. USA 97:3428 (2000); Xiao et al., J. Virol. 72:2224 (1998); Halbert et al., J. Virol. 74:1524 (2000); Halbert et al., J. Virol. 75:6615 (2001); and Auricchio et al., Hum. Molec. Genet. 10:3075 (2001), the disclosures of each of which are incorporated herein by reference as they pertain to AAV vectors for gene delivery.

Also useful in conjunction with the compositions and methods described herein are pseudotyped rAAV vectors. Pseudotyped vectors include AAV vectors of a given serotype (e.g., AAV9) pseudotyped with a capsid gene derived from a serotype other than the given serotype (e.g., AAV1, AAV2, AAV3, AAV4, AAV5, AAV6, AAV7, AAV8, etc.). For example, a representative pseudotyped vector is an AAV8 or AAV9 vector encoding a therapeutic protein (e.g., frataxin) pseudotyped with a capsid gene derived from AAV serotype 2. Techniques involving the construction and use of pseudotyped rAAV virions are known in the art and are described, for example, in Duan et al., J. Virol. 75:7662 (2001); Halbert et al., J. Virol. 74:1524 (2000); Zolotukhin et al., Methods, 28:158 (2002); and Auricchio et al., Hum. Molec. Genet. 10:3075 (2001).

AAV virions that have mutations within the virion capsid may be used to infect particular cell types more effectively than non-mutated capsid virions. For example, suitable AAV mutants may have ligand insertion mutations for the facilitation of targeting AAV to specific cell types. The construction and characterization of AAV capsid mutants including insertion mutants, alanine screening mutants, and epitope tag mutants is described in Wu et al., J. Virol. 74:8635 (2000). Other rAAV virions that can be used in methods described herein include those capsid hybrids that are generated by molecular breeding of viruses as well as by exon shuffling. See, e.g., Soong et al., Nat. Genet., 25:436 (2000) and Kolman and Stemmer, Nat. Biotechnol. 19:423 (2001).

### Exemplary AAV Vectors

As described herein, exemplary AAV vector components may include a promoter, an intron, a polynucleotide encoding PGRN or a codon-optimized PGRN thereof, a 3′ enhancer element, and/or a bovine growth hormone (bGH) polyadenylation site (pA).

In some embodiments, the AAV may include a synapsin promoter, a tetracycline-controlled transactivator protein (tTA) promoter, a reverse tetracycline-controlled transactivator protein (rTA) promoter, a U1 promoter, a U6 promoter, a U7 promoter, a prion promoter, a phosphoglycerate kinase (PGK) promoter, a CB7 promoter, an H1 promoter, a cytomegalovirus (CMV) promoter, a CMV-chicken β-actin (CBA) promoter, a glial fibrillary acidic protein (GFAP) promoter, a calcium/calmodulin-dependent protein kinase III promoter, a tubulin alpha I promoter, a microtubulin-associated protein IB (MAP IB) promoter, a neuron-specific enolase promoter, a platelet-derived growth factor beta chain promoter, a neurofilament light chain promoter, a neuron-specific VGF gene promoter, a neuronal nuclei (NeuN) promoter, a adenomatous polyposis coli (APC) promoter, an ionized calcium-binding adapter molecule 1 (Iba-1) promoter, or a homeobox protein 9 (HB9) promoter. For example, in some embodiments, the promoter is a synapsin promoter. In some embodiments, the PGRN is operably linked to a promoter that is active in a neuronal cell and/or a glial cell.

In some embodiments, the synapsin promoter has a nucleic acid sequence that is at least 85% (e.g., 85%, 86%, 87%, 88%, 89%, 90%, 91%, 92%, 93%, 94%, 95%, 96%, 97%, 98%, or 99%) identical to the nucleic acid sequence of SEQ ID NO: 1. For example, in some embodiments, the synapsin promoter has a nucleic acid sequence that is at least 86% identical to the nucleic acid sequence of SEQ ID NO: 1. In some embodiments, the synapsin promoter has a nucleic acid sequence that is at least 87% identical to the nucleic acid sequence of SEQ ID NO: 1. In some embodiments, the synapsin promoter has a nucleic acid sequence that is at least 88% identical to the nucleic acid sequence of SEQ ID NO: 1. In some embodiments, the synapsin promoter has a nucleic acid sequence that is at least 89% identical to the nucleic acid sequence of SEQ ID NO: 1. In some embodiments, the synapsin promoter has a nucleic acid sequence that is at least 90% identical to the nucleic acid sequence of synapsin ID NO: 1. In some embodiments, the synapsin promoter has a nucleic acid sequence that is at least 91% identical to the nucleic acid sequence of SEQ ID NO: 1. In some embodiments, the synapsin promoter has a nucleic acid sequence that is at least 92% identical to the nucleic acid sequence of SEQ ID NO: 1. In some embodiments, the synapsin promoter has a nucleic acid sequence that is at least 93% identical to the nucleic acid sequence of SEQ ID NO: 1. In some embodiments, the synapsin promoter has a nucleic acid sequence that is at least 94% identical to the nucleic acid sequence of SEQ ID NO: 1. In some embodiments, the synapsin promoter has a nucleic acid sequence that is at least 95% identical to the nucleic acid sequence of SEQ ID NO: 1. In some embodiments, the synapsin promoter has a nucleic acid sequence that is at least 96% identical to the nucleic acid sequence of SEQ ID NO: 1. In some embodiments, the synapsin promoter has a nucleic acid sequence that is at least 97% identical to the nucleic acid sequence of SEQ ID NO: 1. In some embodiments, the synapsin promoter has a nucleic acid sequence that is at least 98% identical to the nucleic acid sequence of SEQ ID NO: 1. In some embodiments, the synapsin promoter has a nucleic acid sequence that is at least 99% identical to the nucleic acid sequence of SEQ ID NO: 1. In some embodiments, the synapsin promoter has a nucleic acid sequence that is identical to the nucleic acid sequence of SEQ ID NO: 1.

In some embodiments, the PGRN is operably linked to a human growth hormone (hGH) intron. For example, in some embodiments, the hGH intron is an hGH intron 3.

In some embodiments, the hGH intron has a nucleic acid sequence that is at least 85% (e.g., 85%, 86%, 87%, 88%, 89%, 90%, 91%, 92%, 93%, 94%, 95%, 96%, 97%, 98%, or 99%) identical to the nucleic acid sequence of SEQ ID NO: 4. For example, in some embodiments, the hGH intron has a nucleic acid sequence that is at least 86% identical to the nucleic acid sequence of SEQ ID NO: 4. In some embodiments, the hGH intron has a nucleic acid sequence that is at least 87% identical to the nucleic acid sequence of SEQ ID NO: 4. In some embodiments, the hGH intron has a nucleic acid sequence that is at least 88% identical to the nucleic acid sequence of SEQ ID NO: 4. In some embodiments, the hGH intron has a nucleic acid sequence that is at least 89% identical to the nucleic acid sequence of SEQ ID NO: 4. In some embodiments, the hGH intron has a nucleic acid sequence that is at least 90% identical to the nucleic acid sequence of ID NO: 1. In some embodiments, the hGH intron has a nucleic acid sequence that is at least 91% identical to the nucleic acid sequence of SEQ ID NO: 4. In some embodiments, the hGH intron has a nucleic acid sequence that is at least 92% identical to the nucleic acid sequence of SEQ ID NO: 4. In some embodiments, the hGH intron has a nucleic acid sequence that is at least 93% identical to the nucleic acid sequence of SEQ ID NO: 4. In some embodiments, the hGH intron has a nucleic acid sequence that is at least 94% identical to the nucleic acid sequence of SEQ ID NO: 4. In some embodiments, the hGH intron has a nucleic acid sequence that is at least 95% identical to the nucleic acid sequence of SEQ ID NO: 4. In some embodiments, the hGH intron has a nucleic acid sequence that is at least 96% identical to the nucleic acid sequence of SEQ ID NO: 4. In some embodiments, the hGH intron has a nucleic acid sequence that is at least 97% identical to the nucleic acid sequence of SEQ ID NO: 4. In some embodiments, the hGH intron has a nucleic acid sequence that is at least 98% identical to the nucleic acid sequence of SEQ ID NO: 4. In some embodiments, the hGH intron has a nucleic acid sequence that is at least 99% identical to the nucleic acid sequence of SEQ ID NO: 4. In some embodiments, the hGH intron has a nucleic acid sequence that is identical to the nucleic acid sequence of SEQ ID NO: 4.

In some embodiments, the AAV may include a PGRN that has an amino acid sequence that is at least 85% (e.g., 85%, 86%, 87%, 88%, 89%, 90%, 91%, 92%, 93%, 94%, 95%, 96%, 97%, 98%, or 99%) identical to the amino acid sequence of SEQ ID NO: 2. For example, in some embodiments, the PGRN has an amino acid sequence that is at least 86% identical to the amino acid sequence of SEQ ID NO: 2. In some embodiments, the PGRN has an amino acid sequence that is at least 87% identical to the amino acid sequence of SEQ ID NO: 2. In some embodiments, the PGRN has an amino acid sequence that is at least 88% identical to the amino acid sequence of SEQ ID NO: 2. In some embodiments, the PGRN has an amino acid sequence that is at least 89% identical to the amino acid sequence of SEQ ID NO: 2. In some embodiments, the PGRN has an amino acid sequence that is at least 90% identical to the amino acid sequence of SEQ ID NO: 2. In some embodiments, THE PGRN has an amino acid sequence that is at least 91% identical to the amino acid sequence of SEQ ID NO: 2. In some embodiments, the PGRN has an amino acid sequence that is at least 92% identical to the amino acid sequence of SEQ ID NO: 2. In some embodiments, the PGRN has an amino acid sequence that is at least 93% identical to the amino acid sequence of SEQ ID NO: 2. In some embodiments, the PGRN has an amino acid sequence that is at least 94% identical to the amino acid sequence of SEQ ID NO: 2. In some embodiments, the PGRN has an amino acid sequence that is at least 95% identical to the amino acid sequence of SEQ ID NO: 2. In some embodiments, the PGRN has an amino acid sequence that is at least 96% identical to the amino acid sequence of SEQ ID NO: 2. In some embodiments, the PGRN has an amino acid sequence that is at least 97% identical to the amino acid sequence of SEQ ID NO: 2. In some embodiments, the PGRN has an amino acid sequence that is at least 98% identical to the amino acid sequence of SEQ ID NO: 2. In some embodiments, the PGRN has an amino acid sequence that is at least 99% identical to the amino acid sequence of SEQ ID NO: 2. In some embodiments, the PGRN encodes a protein that is identical to the amino acid sequence of SEQ ID NO: 2.

In some embodiments, the AAV may include a codon-optimized PGRN that has a nucleic acid sequence that is at least 85% (e.g., 85%, 86%, 87%, 88%, 89%, 90%, 91%, 92%, 93%, 94%, 95%, 96%, 97%, 98%, or 99%) identical to the nucleic acid sequence of SEQ ID NO: 3. For example, in some embodiments, the PGRN has a nucleic acid sequence that is at least 86% identical to the nucleic acid sequence of SEQ ID NO: 3. In some embodiments, the PGRN has a nucleic acid sequence that is at least 87% identical to the nucleic acid sequence of SEQ ID NO: 3. In some embodiments, the PGRN has a nucleic acid sequence that is at least 88% identical to the nucleic acid sequence of SEQ ID NO: 3. In some embodiments, the PGRN has a nucleic acid sequence that is at least 89% identical to the nucleic acid sequence of SEQ ID NO: 3. In some embodiments, the PGRN has a nucleic acid sequence that is at least 90% identical to the nucleic acid sequence of SEQ ID NO: 3. In some embodiments, THE PGRN has a nucleic acid sequence that is at least 91% identical to the nucleic acid sequence of SEQ ID NO: 3. In some embodiments, the PGRN has a nucleic acid sequence that is at least 92% identical to the nucleic acid sequence of SEQ ID NO: 3. In some embodiments, the PGRN has a nucleic acid sequence that is at least 93% identical to the nucleic acid sequence of SEQ ID NO: 3. In some embodiments, the PGRN has a nucleic acid sequence that is at least 94% identical to the nucleic acid sequence of SEQ ID NO: 3. In some embodiments, the PGRN has a nucleic acid sequence that is at least 95% identical to the nucleic acid sequence of SEQ ID NO: 3. In some embodiments, the PGRN has a nucleic acid sequence that is at least 96% identical to the nucleic acid sequence of SEQ ID NO: 3. In some embodiments, the PGRN has a nucleic acid sequence that is at least 97% identical to the nucleic acid sequence of SEQ ID NO: 3. In some embodiments, the PGRN has a nucleic acid sequence that is at least 98% identical to the nucleic acid sequence of SEQ ID NO: 3. In some embodiments, the PGRN has a nucleic acid sequence that is at least 99% identical to the nucleic acid sequence of SEQ ID NO: 3. In some embodiments, the PGRN encodes a protein that is identical to the nucleic acid sequence of SEQ ID NO: 3.

In some embodiments, the PGRN is operably linked to 3′ enhancer element. In some embodiments, the 3′ enhancer element is a human PGRN 3′ untranslated region (UTR).

In some embodiments, the human PGRN 3′ UTR has a nucleic acid sequence that is at least 85% (e.g., 85%, 86%, 87%, 88%, 89%, 90%, 91%, 92%, 93%, 94%, 95%, 96%, 97%, 98%, or 99%) identical to the nucleic acid sequence of SEQ ID NO: 5. For example, in some embodiments, the human PGRN 3′ UTR has a nucleic acid sequence that is at least 86% identical to the nucleic acid sequence of SEQ ID NO: 5. In some embodiments, the human PGRN 3′ UTR has a nucleic acid sequence that is at least 87% identical to the nucleic acid sequence of SEQ ID NO: 5. In some embodiments, the human PGRN 3′ UTR has a nucleic acid sequence that is at least 88% identical to the nucleic acid sequence of SEQ ID NO: 5. In some embodiments, the human PGRN 3′ UTR has a nucleic acid sequence that is at least 89% identical to the nucleic acid sequence of SEQ ID NO: 5. In some embodiments, the human PGRN 3′ UTR has a nucleic acid sequence that is at least 90% identical to the nucleic acid sequence of SEQ ID NO: 5. In some embodiments, the human PGRN 3′ UTR has a nucleic acid sequence that is at least 91% identical to the nucleic acid sequence of SEQ ID NO: 5. In some embodiments, the human PGRN 3′ UTR has a nucleic acid sequence that is at least 92% identical to the nucleic acid sequence of SEQ ID NO: 5. In some embodiments, the human PGRN 3′ UTR has a nucleic acid sequence that is at least 93% identical to the nucleic acid sequence of SEQ ID NO: 5. In some embodiments, the human PGRN 3′ UTR has a nucleic acid sequence that is at least 94% identical to the nucleic acid sequence of SEQ ID NO: 5. In some embodiments, the human PGRN 3′ UTR has a nucleic acid sequence that is at least 95% identical to the nucleic acid sequence of SEQ ID NO: 5. In some embodiments, the human PGRN 3′ UTR has a nucleic acid sequence that is at least 96% identical to the nucleic acid sequence of SEQ ID NO: 5. In some embodiments, the human PGRN 3′ UTR has a nucleic acid sequence that is at least 97% identical to the nucleic acid sequence of SEQ ID NO: 5. In some embodiments, the human PGRN 3′ UTR has a nucleic acid sequence that is at least 98% identical to the nucleic acid sequence of SEQ ID NO: 5. In some embodiments, the human PGRN 3′ UTR has a nucleic acid sequence that is at least 99% identical to the nucleic acid sequence of SEQ ID NO: 5. In some embodiments, the human PGRN 3′ UTR has a nucleic acid sequence that is identical to the nucleic acid sequence of SEQ ID NO: 5.

In some embodiments, the PGRN is operably linked to a bGH pA.

In some embodiments, the bGH pA has a nucleic acid sequence that is at least 85% (e.g., 85%, 86%, 87%, 88%, 89%, 90%, 91%, 92%, 93%, 94%, 95%, 96%, 97%, 98%, or 99%) identical to the nucleic acid sequence of SEQ ID NO: 8. For example, in some embodiments, the bGH pA has a nucleic acid sequence that is at least 86% identical to the nucleic acid sequence of SEQ ID NO: 8. In some embodiments, the bGH pA has a nucleic acid sequence that is at least 87% identical to the nucleic acid sequence of SEQ ID NO: 8. In some embodiments, the bGH pA has a nucleic acid sequence that is at least 88% identical to the nucleic acid sequence of SEQ ID NO: 8. In some embodiments, the bGH pA has a nucleic acid sequence that is at least 89% identical to the nucleic acid sequence of SEQ ID NO: 8. In some embodiments, the bGH pA has a nucleic acid sequence that is at least 90% identical to the nucleic acid sequence of SEQ ID NO: 8. In some embodiments, the bGH pA has a nucleic acid sequence that is at least 91% identical to the nucleic acid sequence of SEQ ID NO: 8. In some embodiments, the bGH pA has a nucleic acid sequence that is at least 92% identical to the nucleic acid sequence of SEQ ID NO: 8. In some embodiments, the bGH pA has a nucleic acid sequence that is at least 93% identical to the nucleic acid sequence of SEQ ID NO: 8. In some embodiments, the bGH pA has a nucleic acid sequence that is at least 94% identical to the nucleic acid sequence of SEQ ID NO: 8. In some embodiments, the bGH pA has a nucleic acid sequence that is at least 95% identical to the nucleic acid sequence of SEQ ID NO: 8. In some embodiments, the bGH pA has a nucleic acid sequence that is at least 96% identical to the nucleic acid sequence of SEQ ID NO: 8. In some embodiments, the bGH pA has a nucleic acid sequence that is at least 97% identical to the nucleic acid sequence of SEQ ID NO: 8. In some embodiments, the bGH pA has a nucleic acid sequence that is at least 98% identical to the nucleic acid sequence of SEQ ID NO: 8. In some embodiments, the bGH pA has a nucleic acid sequence that is at least 99% identical to the nucleic acid sequence of SEQ ID NO: 8. In some embodiments, the bGH pA has a nucleic acid sequence that is identical to the nucleic acid sequence of SEQ ID NO: 8.

Exemplary nucleic acids that can be incorporated into an AAV are described in Table 4, below.

TABLE 4
Exemplary polynucleotides useful for incorporation into an AAV

| SEQ ID NO. | Description | Sequence |
|---|---|---|
| 1 | Human | AGTGCAAGTGGGTTTTAGGACCAGGATGAGGCGGGGTGGGGGT |
| | synapsin | GCCTACCTGACGACCGACCCCGACCCACTGGACAAGCACCCAAC |
| | promoter | CCCCATTCCCCAAATTGCGCATCCCCTATCAGAGAGGGGGAGGG |
| | | GAAACAGGATGCGGCGAGGCGCGTGCGCACTGCCAGCTTCAGC |
| | | ACCGCGGACAGTGCCTTCGCCCCCGCCTGGCGGCGCGCGCCAC |
| | | CGCCGCCTCAGCACTGAAGGCGCGCTGACGTCACTCGCCGGTC |
| | | CCCCGCAAACTCCCCTTCCCGGCCACCTTGGTCGCGTCCGCGCC |
| | | GCCGCCGGCCCAGCCGGACCGCACCACGCGAGGCGCGAGATA |
| | | GGGGGGCACGGGCGCGACCATCTGCGCTGCGGCGCCGGCGAC |
| | | TCAGCGCTGCCTCAGTCTGCGGTGGGCAGCGGAGGAGTCGTGT |
| | | CGTGCCTGAGAGCGCAG |
| 4 | hGH intron | GTGAGTGGATGCCTTCTCCCCAGGCGGGGATGGGGGAGACCTG |
| | | TAGTCAGAGCCCCCGGGCAGCACAGCCAATGCCCGTCCTTCCCC |
| | | TGCAG |
| 9 | Human | ATTCTCCAATCACATGATCCCTAGAAATGGGGTGTGGGGCGAGA |
| | PGRN 5′ | GGAAGCAGGGAGGAGAGTGATTTGAGTAGAAAAGAAACACAGCA |
| | UTR | TTCCAGGCTGGCCCCACCTCTATATTGATAAGTAGCCAATGGGAG |
| | | CGGGTAGCCCTGATCCCTGGCCAATGGAAACTGAGGTAGGCGG |
| | | GTCATCGCGCTGGGGTCTGTAGTCTGAGCGCTACCCGGTTGCTG |
| | | CTGCCCAAGGACCGCGGAGTCGGACGCAGGCAGACC |
| 7 | Wild-type | TGTGGACCCTGGTGAGCTGGGTGGCCTTAACAGCAGGGCTGGT |
| | human GRN | GGCTGGAACGCGGTGCCCAGATGGTCAGTTCTGCCCTGTGGCCT |
| | | GCTGCCTGGACCCCGGAGGAGCCAGCTACAGCTGCTGCCGTCC |
| | | CCTTCTGGACAAATGGCCCACAACACTGAGCAGGCATCTGGGTG |
| | | GCCCCTGCCAGGTTGATGCCCACTGCTCTGCCGGCCACTCCTGC |
| | | ATCTTTACCGTCTCAGGGACTTCCAGTTGCTGCCCCTTCCCAGAG |
| | | GCCGTGGCATGCGGGGATGGCCATCACTGCTGCCCACGGGGCT |
| | | TCCACTGCAGTGCAGACGGGCGATCCTGCTTCCAAAGATCAGGT |
| | | AACAACTCCGTGGGTGCCATCCAGTGCCCTGATAGTCAGTTCGA |
| | | ATGCCCGGACTTCTCCACGTGCTGTGTTATGGTCGATGGCTCCT |
| | | GGGGGTGCTGCCCCATGCCCCAGGCTTCCTGCTGTGAAGACAG |
| | | GGTGCACTGCTGTCCGCACGGTGCCTTCTGCGACCTGGTTCACA |
| | | CCCGCTGCATCACACCCACGGGCACCCACCCCCTGGCAAAGAA |
| | | GCTCCCTGCCCAGAGGACTAACAGGGCAGTGGCCTTGTCCAGCT |
| | | CGGTCATGTGTCCGGACGCACGGTCCCGGTGCCCTGATGGTTCT |
| | | ACCTGCTGTGAGCTGCCCAGTGGGAAGTATGGCTGCTGCCCAAT |
| | | GCCCAACGCCACCTGCTGCTCCGATCACCTGCACTGCTGCCCCC |
| | | AAGACACTGTGTGTGACCTGATCCAGAGTAAGTGCCTCTCCAAG |
| | | GAGAACGCTACCACGGACCTCCTCACTAAGCTGCCTGCGCACAC |
| | | AGTGGGGGATGTGAAATGTGACATGGAGGTGAGCTGCCCAGATG |
| | | GCTATACCTGCTGCCGTCTACAGTCGGGGGCCTGGGGCTGCTG |
| | | CCCTTTTACCCAGGCTGTGTGCTGTGAGGACCACATACACTGCT |
| | | GTCCCGCGGGGTTTACGTGTGACACGCAGAAGGGTACCTGTGAA |
| | | CAGGGGCCCCACCAGGTGCCCTGGATGGAGAAGGCCCCAGCTC |
| | | ACCTCAGCCTGCCAGACCCACAAGCCTTGAAGAGAGATGTCCCC |
| | | TGTGATAATGTCAGCAGCTGTCCCTCCTCCGATACCTGCTGCCAA |
| | | CTCACGTCTGGGGAGTGGGGCTGCTGTCCAATCCCAGAGGCTGT |
| | | CTGCTGCTCGGACCACCAGCACTGCTGCCCCCAGGGCTACACGT |
| | | GTGTAGCTGAGGGGCAGTGTCAGCGAGGAAGCGAGATCGTGGC |
| | | TGGACTGGAGAAGATGCCTGCCCGCCGGGCTTCCTTATCCCACC |
| | | CCAGAGACATCGGCTGTGACCAGCACACCAGCTGCCCGGTGGG |
| | | GCAGACCTGCTGCCCGAGCCTGGGGGGAGCTGGGCCTGCTGC |
| | | CAGTTGCCCCATGCTGTGTGCTGCGAGGATCGCCAGCACTGCTG |
| | | CCCGGCTGGCTACACCTGCAACGTGAAGGCTCGATCCTGCGAGA |
| | | AGGAAGTGGTCTCTGCCCAGCCTGCCACCTTCCTGGCCCGTAGC |
| | | CCTCACGTGGGTGTGAAGGACGTGGAGTGTGGGGAAGGACACT |
| | | TCTGCCATGATAACCAGACCTGCTGCCGAGACAACCGACAGGGC |
| | | TGGGCCTGCTGTCCCTACCGCCAGGGCGTCTGTTGTGCTGATCG |
| | | GCGCCACTGCTGTCCTGCTGGCTTCCGCTGCGCAGCCAGGGGT |
| | | ACCAAGTGTTTGCGCAGGGAGGCCCCGCGCTGGGACGCCCCTT |
| | | TGAGGGACCCAGCCTTGAGACAGCTGCTGTGA |
| 3 | Codon- | ATGTGGACTCTGGTCTCCTGGGTCGCTCTGACCGCTGGCCTGGT |
| | optimized | CGCTGGGACAAGATGCCCCGATGGACAGTTTTGCCCCGTCGCTT |
| | human GRN | GCTGTCTGGACCCAGGAGGAGCCAGCTACTCCTGCTGTCGGCCA |
| | | CTGCTGGATAAGTGGCCCACCACACTGTCCCGCCACCTGGGAGG |
| | | ACCATGCCAGGTGGACGCACACTGTTCCGCCGGACACTCTTGCA |
| | | TCTTCACAGTGTCTGGCACCAGCTCCTGCTGTCCATTTCCTGAGG |
| | | CAGTGGCATGCGGCGACGGACACCACTGCTGTCCCAGGGGCTT |
| | | CCACTGTAGCGCCGATGGCAGGTCCTGCTTTCAGAGAAGCGGCA |
| | | ACAATTCCGTGGGCGCCATCCAGTGTCCTGACAGCCAGTTCGAA |
| | | TGCCCAGATTTTTCCACCTGCTGCGTGATGGTGGACGGCTCTTG |
| | | GGGCTGCTGTCCAATGCCACAGGCCAGCTGCTGTGAGGACAGG |
| | | GTGCACTGCTGTCCTCACGGAGCCTTCTGTGATCTGGTGCACAC |
| | | ACGCTGCATCACCCCCACAGGCACCCACCCTCTGGCCAAGAAGC |
| | | TGCCAGCACAGAGGACCAACAGGGCAGTGGCCCTGAGCAGCAG |
| | | CGTGATGTGCCCCGACGCCAGGTCTAGATGCCCTGATGGCAGCA |
| | | CCTGCTGTGAGCTGCCAAGCGGCAAGTACGGCTGCTGTCCTATG |
| | | CCAAACGCCACATGCTGTTCCGACCACCTGCACTGCTGTCCTCA |
| | | GGACACCGTGTGCGATCTGATCCAGTCTAAGTGCCTGAGCAAGG |
| | | AGAATGCCACCACAGACCTGCTGACAAAGCTGCCTGCCCACACC |
| | | GTGGGCGACGTGAAGTGTGATATGGAGGTGTCCTGCCCAGATGG |
| | | CTATACATGCTGTAGGCTGCAGTCTGGAGCATGGGGATGCTGTC |
| | | CCTTCACCCAGGCCGTGTGCTGTGAGGACCACATCCACTGCTGT |
| | | CCTGCCGGCTTTACATGTGATACCCAGAAGGGCACATGCGAGCA |
| | | GGGCCCTCACCAGGTGCCATGGATGGAGAAGGCACCAGCACAC |
| | | CTGTCCCTGCCCGACCCTCAGGCCCTGAAGAGAGACGTGCCTTG |
| | | TGATAACGTGTCTAGCTGCCCATCCTCTGATACATGCTGTCAGCT |
| | | GACCTCTGGCGAGTGGGGCTGCTGTCCAATCCCCGAGGCCGTG |
| | | TGCTGTAGCGACCACCAGCACTGCTGTCCTCAGGGCTATACCTG |
| | | CGTGGCAGAGGGACAGTGCCAGAGGGGCTCCGAGATCGTGGCA |
| | | GGCCTGGAGAAGATGCCAGCCAGGAGAGCCTCTCTGAGCCACC |
| | | CCAGAGACATCGGCTGTGATCAGCACACAAGCTGCCCAGTGGGA |
| | | CAGACCTGCTGTCCATCCCTGGGAGGCTCTTGGGCATGCTGTCA |
| | | GCTGCCTCACGCCGTGTGCTGTGAGGATAGGCAGCACTGCTGTC |
| | | CAGCCGGCTACACATGCAATGTGAAGGCCAGATCCTGCGAGAAG |
| | | GAGGTGGTGTCTGCCCAGCCAGCCACCTTCCTGGCACGCAGCC |
| | | CTCACGTGGGCGTGAAGGACGTGGAGTGTGGCGAGGGCCACTT |
| | | TTGCCACGACAACCAGACATGCTGTAGGGATAATAGACAGGGCT |
| | | GGGCCTGCTGTCCATATAGGCAGGGCGTGTGCTGTGCAGATCG |
| | | GCGCCACTGCTGTCCAGCAGGCTTTCGGTGCGCAGCCAGGGGC |
| | | ACCAAGTGCCTGCGCAGAGAAGCCCCCCGGTGGGACGCCCCCC |
| | | TGCGAGACCCCGCCCTGAGACAGCTGCTGTGA |
| 5 | hPGRN 3′ | GGGACAGTACTGAAGACTCTGCAGCCCTCGGGACCCCACTCGGA |
| | UTR | GGGTGCCCTCTGCTCAGGCCTCCCTAGCACCTCCCCCTAACCAA |
| | | ATTCTCCCTGGACCCCATTCTGAGCTCCCCATCACCATGGGAGG |
| | | TGGGGCCTCAATCTAAGGCCTTCCCTGTCAGAAGGGGGTTGTGG |
| | | CAAAAGCCACATTACAAGCTGCCATCCCCTCCCCGTTTCAGTGGA |
| | | CCCTGTGGCCAGGTGCTTTTCCCTATCCACAGGGGTGTTTGTGT |
| | | GTGTGCGCGTGTGCGTTTC |
| 8 | bGH pA | CTGTGCCTTCTAGTTGCCAGCCATCTGTTGTTTGCCCCTCCCCCG |
| | | TGCCTTCCTTGACCCTGGAAGGTGCCACTCCCACTGTCCTTTCCT |
| | | AATAAAATGAGGAAATTGCATCGCATTGTCTGAGTAGGTGTCATT |
| | | CTATTCTGGGGGGTGGGGTGGGGCAGGACAGCAAGGGGGAGGA |
| | | TTGGGAAGACAATAGCAGGCATGCTGGGGA |

In some embodiments, the AAV has a nucleic acid sequence that is at least 85% (e.g., 85%, 86%, 87%, 88%, 89%, 90%, 91%, 92%, 93%, 94%, 95%, 96%, 97%, 98%, or 99%) identical to the nucleic acid sequence of SEQ ID NO: 6. For example, in some embodiments, the AAV has a nucleic acid sequence that is at least 86% identical to the nucleic acid sequence of SEQ ID NO: 6. In some embodiments, the AAV has a nucleic acid sequence that is at least 87% identical to the nucleic acid sequence of SEQ ID NO: 6. In some embodiments, the AAV has a nucleic acid sequence that is at least 88% identical to the nucleic acid sequence of SEQ ID NO: 6. In some embodiments, the AAV has a nucleic acid sequence that is at least 89% identical to the nucleic acid sequence of SEQ ID NO: 6. In some embodiments, the AAV has a nucleic acid sequence that is at least 90% identical to the nucleic acid sequence of SEQ ID NO: 6. In some embodiments, the AAV has a nucleic acid sequence that is at least 91% identical to the nucleic acid sequence of SEQ ID NO: 6. In some embodiments, the AAV has a nucleic acid sequence that is at least 92% identical to the nucleic acid sequence of SEQ ID NO: 6. In some embodiments, the AAV has a nucleic acid sequence that is at least 93% identical to the nucleic acid sequence of SEQ ID NO: 6. In some embodiments, the AAV has a nucleic acid sequence that is at least 94% identical to the nucleic acid sequence of SEQ ID NO: 6. In some embodiments, the AAV has a nucleic acid sequence that is at least 95% identical to the nucleic acid sequence of SEQ ID NO: 6. In some embodiments, the AAV has a nucleic acid sequence that is at least 96% identical to the nucleic acid sequence of SEQ ID NO: 6. In some embodiments, the AAV has a nucleic acid sequence that is at least 97% identical to the nucleic acid sequence of SEQ ID NO: 6. In some embodiments, the AAV has a nucleic acid sequence that is at least 98% identical to the nucleic acid sequence of SEQ ID NO: 6. In some embodiments, the AAV has a nucleic acid sequence that is at least 99% identical to the nucleic acid sequence of SEQ ID NO: 6. In some embodiments, the AAV has a nucleic acid sequence that is identical to the nucleic acid sequence of SEQ ID NO: 6.

As described herein, an exemplary AAV having the nucleic acid sequence of SEQ ID NO: 6, is shown below:

CCTGCAGGCAGCTGCGCGCTCGCTCGCTCACTGAGGCCGCCCGGGCGTCGGGCGACC
TTTGGTCGCCCGGCCTCAGTGAGCGAGCGAGCGCGCAGAGAGGGAGTGGCCAACTCC
ATCACTAGGGGTTCCTGCGGCCGCACGCGTTGCAAAGATGGATAAAGTTTTAAACAGAG
AGGAATCTTTGCAGCTAATGGACCTTCTAGGTCTTGAAAGGAGTGGGAATTGGCTCCGG
TGCCCGTCAGTGGGCAGAGCGCACATCGCCCACAGTCCCCGAGAAGTTGGGGGGAGG
GGTCGGCAGCAAATGGTTAATTAATCTAGACTGCAGAGGGCCCTGCGTATGAGTGCAAG
TGGGTTTTAGGACCAGGATGAGGCGGGGTGGGGGTGCCTACCTGACGACCGACCCCG
ACCCACTGGACAAGCACCCAACCCCCATTCCCCAAATTGCGCATCCCCTATCAGAGAGG
GGGAGGGGAAACAGGATGCGGCGAGGCGCGTGCGCACTGCCAGCTTCAGCACCGCGG
ACAGTGCCTTCGCCCCCGCCTGGCGGCGCGCGCCACCGCCGCCTCAGCACTGAAGGC
GCGCTGACGTCACTCGCCGGTCCCCCGCAAACTCCCCTTCCCGGCCACCTTGGTCGCG
TCCGCGCCGCCGCCGGCCCAGCCGGACCGCACCACGCGAGGCGCGAGATAGGGGGG
CACGGGCGCGACCATCTGCGCTGCGGCGCCGGCGACTCAGCGCTGCCTCAGTCTGCG
GTGGGCAGCGGAGGAGTCGTGTCGTGCCTGAGAGCGCAGTCGAGAGGATCCGTGAGT
GGATGCCTTCTCCCCAGGCGGGGATGGGGGAGACCTGTAGTCAGAGCCCCCGGGCAG
CACAGCCAATGCCCGTCCTTCCCCTGCAGACCGGTGCCACCATGTGGACTCTGGTCTCC
TGGGTCGCTCTGACCGCTGGCCTGGTCGCTGGGACAAGATGCCCCGATGGACAGTTTT
GCCCCGTCGCTTGCTGTCTGGACCCAGGAGGAGCCAGCTACTCCTGCTGTCGGCCACT
GCTGGATAAGTGGCCCACCACACTGTCCCGCCACCTGGGAGGACCATGCCAGGTGGAC
GCACACTGTTCCGCCGGACACTCTTGCATCTTCACAGTGTCTGGCACCAGCTCCTGCTG
TCCATTTCCTGAGGCAGTGGCATGCGGCGACGGACACCACTGCTGTCCCAGGGGCTTC
CACTGTAGCGCCGATGGCAGGTCCTGCTTTCAGAGAAGCGGCAACAATTCCGTGGGCG
CCATCCAGTGTCCTGACAGCCAGTTCGAATGCCCAGATTTTTCCACCTGCTGCGTGATG
GTGGACGGCTCTTGGGGCTGCTGTCCAATGCCACAGGCCAGCTGCTGTGAGGACAGGG
TGCACTGCTGTCCTCACGGAGCCTTCTGTGATCTGGTGCACACACGCTGCATCACCCCC
ACAGGCACCCACCCTCTGGCCAAGAAGCTGCCAGCACAGAGGACCAACAGGGCAGTGG
CCCTGAGCAGCAGCGTGATGTGCCCCGACGCCAGGTCTAGATGCCCTGATGGCAGCAC
CTGCTGTGAGCTGCCAAGCGGCAAGTACGGCTGCTGTCCTATGCCAAACGCCACATGCT
GTTCCGACCACCTGCACTGCTGTCCTCAGGACACCGTGTGCGATCTGATCCAGTCTAAG
TGCCTGAGCAAGGAGAATGCCACCACAGACCTGCTGACAAAGCTGCCTGCCCACACCG
TGGGCGACGTGAAGTGTGATATGGAGGTGTCCTGCCCAGATGGCTATACATGCTGTAGG
CTGCAGTCTGGAGCATGGGGATGCTGTCCCTTCACCCAGGCCGTGTGCTGTGAGGACC
ACATCCACTGCTGTCCTGCCGGCTTTACATGTGATACCCAGAAGGGCACATGCGAGCAG
GGCCCTCACCAGGTGCCATGGATGGAGAAGGCACCAGCACACCTGTCCCTGCCCGACC
CTCAGGCCCTGAAGAGAGACGTGCCTTGTGATAACGTGTCTAGCTGCCCATCCTCTGAT
ACATGCTGTCAGCTGACCTCTGGCGAGTGGGGCTGCTGTCCAATCCCCGAGGCCGTGT
GCTGTAGCGACCACCAGCACTGCTGTCCTCAGGGCTATACCTGCGTGGCAGAGGGACA
GTGCCAGAGGGGCTCCGAGATCGTGGCAGGCCTGGAGAAGATGCCAGCCAGGAGAGC
CTCTCTGAGCCACCCCAGAGACATCGGCTGTGATCAGCACACAAGCTGCCCAGTGGGA
CAGACCTGCTGTCCATCCCTGGGAGGCTCTTGGGCATGCTGTCAGCTGCCTCACGCCG
TGTGCTGTGAGGATAGGCAGCACTGCTGTCCAGCCGGCTACACATGCAATGTGAAGGC
CAGATCCTGCGAGAAGGAGGTGGTGTCTGCCCAGCCAGCCACCTTCCTGGCACGCAGC
CCTCACGTGGGCGTGAAGGACGTGGAGTGTGGCGAGGGCCACTTTTGCCACGACAACC
AGACATGCTGTAGGGATAATAGACAGGGCTGGGCCTGCTGTCCATATAGGCAGGGCGT
GTGCTGTGCAGATCGGCGCCACTGCTGTCCAGCAGGCTTTCGGTGCGCAGCCAGGGGC
ACCAAGTGCCTGCGCAGAGAAGCCCCCCGGTGGGACGCCCCCCTGCGAGACCCCGCC
CTGAGACAGCTGCTGTGAGTCGCTGGTTTAAACGGGACAGTACTGAAGACTCTGCAGCC
CTCGGGACCCCACTCGGAGGGTGCCCTCTGCTCAGGCCTCCCTAGCACCTCCCCCTAA
CCAAATTCTCCCTGGACCCCATTCTGAGCTCCCCATCACCATGGGAGGTGGGGCCTCAA
TCTAAGGCCTTCCCTGTCAGAAGGGGGTTGTGGCAAAAGCCACATTACAAGCTGCCATC
CCCTCCCCGTTTCAGTGGACCCTGTGGCCAGGTGCTTTTCCCTATCCACAGGGGTGTTT
GTGTGTGTGCGCGTGTGCGTTTCGCTAGCCTCGAGAGATCGATCTGCCTCGACTGTGCC
TTCTAGTTGCCAGCCATCTGTTGTTTGCCCCTCCCCCGTGCCTTCCTTGACCCTGGAAG
GTGCCACTCCCACTGTCCTTTCCTAATAAAATGAGGAAATTGCATCGCATTGTCTGAGTA
GGTGTCATTCTATTCTGGGGGGTGGGGTGGGGCAGGACAGCAAGGGGGAGGATTGGG
AAGACAATAGCAGGCATGCTGGGGACACGTGCGGACCGAGCGGCCGCAGGAACCCCT
AGTGATGGAGTTGGCCACTCCCTCTCTGCGCGCTCGCTCGCTCACTGAGGCCGGGCGA
CCAAAGGTCGCCCGACGCCCGGGCTTTGCCCGGGCGGCCTCAGTGAGCGAGCGAGCG
CGCAGCTGCCTGCAGGGGCGCCACTAGTTGATGCGGTATTTTCTCCTTACGCATCTGTG
CGGTATTTCACACCGCATACGTCAAAGCAACCATAGTACGCGCCCTGTAGCGGCGCATT
AAGCGCGGCGGGTGTGGTGGTTACGCGCAGCGTGACCGCTACACTTGCCAGCGCCCTA
GCGCCCGCTCCTTTCGCTTTCTTCCCTTCCTTTCTCGCCACGTTCGCCGGCTTTCCCCGT
CAAGCTCTAAATCGGGGGCTCCCTTTAGGGTTCCGATTTAGTGCTTTACGGCACCTCGA
CCCCAAAAAACTTGATTTGGGTGATGGTTCACGTAGTGGGCCATCGCCCTGATAGACGG
TTTTTCGCCCTTTGACGTTGGAGTCCACGTTCTTTAATAGTGGACTCTTGTTCCAAACTG
GAACAACACTCAACCCTATCTCGGGCTATTCTTTTGATTTATAAGGGATTTTGCCGATTTC
GGCCTATTGGTTAAAAAATGAGCTGATTTAACAAAAATTTAACGCGAATTTTAACAAAATA
TTAACGTTTACAATTTTATGGTGCACTCTCAGTACAATCTGCTCTGATGCCGCATAGTTAA
GCCAGCCCCGACACCCGCCAACACCCGCTGACGCGCCCTGACGGGCTTGTCTGCTCCC
GGCATCCGCTTACAGACAAGCTGTGACCGTCTCCGGGAGCTGCATGTGTCAGAGGTTTT
CACCGTCATCACCGAAACGCGCGAGACGAAAGGGCCTCGTGATACGCCTATTTTTATAG
GTTAATGTCATGATAATAATGGTTTCTTAGACGTCAGGTGGCACTTTTCGGGGAAATGTG
CGCGGAACCCCTATTTGTTTATTTTTCTAAATACATTCAAATATGTATCCGCTCATGAGAC
AATAACCCTGATAAATGCTTCAATAATATTGAAAAAGGAAGAGTATGAGCCATATTCAACG
GGAAACGTCGAGGCCGCGATTAAATTCCAACATGGATGCTGATTTATATGGGTATAAATG
GGCTCGCGATAATGTCGGGCAATCAGGTGCGACAATCTATCGCTTGTATGGGAAGCCCG
ATGCGCCAGAGTTGTTTCTGAAACATGGCAAAGGTAGCGTTGCCAATGATGTTACAGAT
GAGATGGTCAGACTAAACTGGCTGACGGAATTTATGCCTCTTCCGACCATCAAGCATTTT
ATCCGTACTCCTGATGATGCATGGTTACTCACCACTGCGATCCCCGGAAAAACAGCATTC
CAGGTATTAGAAGAATATCCTGATTCAGGTGAAAATATTGTTGATGCGCTGGCAGTGTTC
CTGCGCCGGTTGCATTCGATTCCTGTTTGTAATTGTCCTTTTAACAGCGATCGCGTATTT
CGTCTCGCTCAGGCGCAATCACGAATGAATAACGGTTTGGTTGATGCGAGTGATTTTGAT
GACGAGCGTAATGGCTGGCCTGTTGAACAAGTCTGGAAAGAAATGCATAAACTTTTGCC
ATTCTCACCGGATTCAGTCGTCACTCATGGTGATTTCTCACTTGATAACCTTATTTTTGAC
GAGGGGAAATTAATAGGTTGTATTGATGTTGGACGAGTCGGAATCGCAGACCGATACCA
GGATCTTGCCATCCTATGGAACTGCCTCGGTGAGTTTTCTCCTTCATTACAGAAACGGCT
TTTTCAAAAATATGGTATTGATAATCCTGATATGAATAAATTGCAGTTTCATTTGATGCTCG
ATGAGTTTTTCTAATAACTGTCAGACCAAGTTTACTCATATATACTTTAGATTGATTTAAAA
CTTCATTTTTAATTTAAAAGGATCTAGGTGAAGATCCTTTTTGATAATCTCATGACCAAAAT
CCCTTAACGTGAGTTTTCGTTCCACTGAGCGTCAGACCCCGTAGAAAAGATCAAAGGAT
CTTCTTGAGATCCTTTTTTTCTGCGCGTAATCTGCTGCTTGCAAACAAAAAAACCACCGCT
ACCAGCGGTGGTTTGTTTGCCGGATCAAGAGCTACCAACTCTTTTTCCGAAGGTAACTG
GCTTCAGCAGAGCGCAGATACCAAATACTGTTCTTCTAGTGTAGCCGTAGTTAGGCCAC
CACTTCAAGAACTCTGTAGCACCGCCTACATACCTCGCTCTGCTAATCCTGTTACCAGTG
GCTGCTGCCAGTGGCGATAAGTCGTGTCTTACCGGGTTGGACTCAAGACGATAGTTACC
GGATAAGGCGCAGCGGTCGGGCTGAACGGGGGGTTCGTGCACACAGCCCAGCTTGGA
GCGAACGACCTACACCGAACTGAGATACCTACAGCGTGAGCTATGAGAAAGCGCCACG
CTTCCCGAAGGGAGAAAGGCGGACAGGTATCCGGTAAGCGGCAGGGTCGGAACAGGA
GAGCGCACGAGGGAGCTTCCAGGGGGAAACGCCTGGTATCTTTATAGTCCTGTCGGGT
TTCGCCACCTCTGACTTGAGCGTCGATTTTTGTGATGCTCGTCAGGGGGGCGGAGCCTA
TGGAAAAACGCCAGCAACGCGGCCTTTTTACGGTTCCTGGCCTTTTGCTGGCCTTTTGC
TCAGGCCTACATGT

### Additional Therapeutic Transgenes and Proteins

The AAVs described herein may include a polynucleotide encoding a therapeutic protein useful for the treatment of a disorder affecting the CNS (e.g., a neurocognitive or a neuromuscular disorder (e.g., a neurodegenerative disease) or a lysosomal storage disorder, among other disorders that adversely affect the CNS. Exemplary therapeutic proteins useful in conjunction with the compositions and methods of the disclosure are shown in Table 5, below.

TABLE 5
Exemplary therapeutic proteins of the disclosure

| | Disease associated with | Exemplary wild-type or variant |
|---|---|---|
| Protein | deficiency in protein | protein sequence |
| acid α-glucosidase (GAA) | Pompe | NP_000143.2, |
| | | NP_001073271.1, |
| | | NP_001073272.1 |
| Methyl CpG binding protein 2 | Rett syndrome | NP_001104262.1, |
| (MECP2) | | NP_004983.1 |
| Aromatic L-amino acid | Parkinson's disease | NP_000781.1, |
| decarboxylase (AADC) | | NP_001076440.1, |
| | | NP_001229815.1, |
| | | NP_001229816.1, |
| | | NP_001229817.1, |
| | | NP_001229818.1, |
| | | NP_001229819.1 |
| Glial cell-derived neurotrophic | Parkinson's disease | NP_000505.1, |
| factor (GDNF) | | NP_001177397.1, |
| | | NP_001177398.1, |
| | | NP_001265027.1, |
| | | NP_954701.1 |
| Glutamate decarboxylase 1 | Parkinson's disease | NP_000808.2, NP_038473.2 |
| (GAD1) | | |
| Glutamate decarboxylase 2 | Parkinson's disease | NP_000809.1, |
| (GAD2) | | NP_001127838.1 |
| Neurturin (NRTN) | Parkinson's disease | NP_004549.1 |
| neuropeptide Y (NPY) | Parkinson's disease, epilepsy | NP_000896.1 |
| Cystic fibrosis transmembrane | Cystic fibrosis | NP_000483.3 |
| conductance regulator (CFTR) | | |
| Tumor necrosis factor receptor | Arthritis, Rheumatoid arthritis | SEQ ID NO. 1 of |
| fused to an antibody Fe | | W02013025079 |
| (TNFR: Fc) | | |
| Sarcoglycan α, β, γ, Δ, ε, or ζ | Muscular dystrophy | SGCA |
| (SGCA, SGCB, SGCG, SGCD, | | NP_000014.1, |
| SGCE, or SGCZ) | | NP_001129169.1 |
| | | SGCB |
| | | NP_000223.1 |
| | | SGCG |
| | | NP_000222.1 |
| | | SGCD |
| | | NP_000328.2, |
| | | NP_001121681.1, |
| | | NP_758447.1 |
| | | SGCE |
| | | NP_001092870.1, |
| | | NP_001092871.1, |
| | | NP_003910.1 |
| | | SGCZ |
| | | NP_631906.2 |
| α-1-antitrypsin (AAT) | Hereditary emphysema or α-1- | NP_000286.3, |
| | antitrypsin deficiency | NP_001002235.1, |
| | | NP_001002236.1, |
| | | NP_001121172.1, |
| | | NP_001121173.1, |
| | | NP_001121174.1, |
| | | NP_001121175.1, |
| | | NP_001121176.1, |
| | | NP_001121177.1, |
| | | NP_001121178.1, |
| | | NP_001121179.1 |
| Aspartoacylase (ASPA) | Canavan's disease | NP_000040.1, |
| | | NP_001121557.1 |
| Nerve growth factor (NGF) | Alzheimer's disease | NP_002497.2 |
| Granulocyte-macrophage | Prostate cancer | NP_000749.2 |
| colonystimulating factory (GM- | | |
| CSF) | | |
| Cluster of Differentiation 86 | Malignant melanoma | NP_001193853.1, |
| (CD86 or B7-2) | | NP_001193854.1, |
| | | NP_008820.3, |
| | | NP_787058.4, |
| | | NP_795711.1 |
| Interleukin 12 (IL-12) | Malignant melanoma | NP_000873.2, NP_002178.2 |
| ATPase, Ca²⁺ transporting, | Chronic heart failure | NP_001672.1, |
| cardiac muscle, slow twitch 2 | | NP_733765.1 |
| (SERCA2) | | |
| Dystrophin or Minidystrophin | Muscular dystrophy | NP_000100.2, NP_003997.1, |
| | | NP_004000.1, NP_004001.1, |
| | | NP_004002.2, NP_004003.1, |
| | | NP_004004.1, NP_004005.1, |
| | | NP_004006.1, NP_004007.1, |
| | | NP_004008.1, NP_004009.1, |
| | | NP_004010.1, NP_004011.2, |
| | | NP_004012.1, NP_004013.1, |
| | | NP_004014.1 |
| Ceroid lipofuscinosis neuronal 2 | Late infantile neuronal | NP_000382.3 |
| (CLN2) | ceroidlipofuscinosis or Batten's | |
| | disease | |
| N-acetylglucosaminidase, α | Sanfilippo syndrome (MPSIIIB) | NP_000254.2 |
| (NAGLU) | | |
| Iduronidase, α -1 (IDUA) | MPSI-Hurler | NP_000194.2 |
| Iduronate 2-sulfatase (IDS) | MPSII-Hunter | NP_000193.1, |
| | | NP_001160022.1, |
| | | NP_006114.1 |
| Glucuronidase, β (GUSB) | MPSVII-Sly | NP_000172.2, NP 001271219.1 |
| Hexosaminidase A, α | Tay-Sachs | NP_000511.2 |
| polypeptide (HEXA) | | |
| Retinal pigment epithelium- | Leber congenital amaurosis | NP_000320.1 |
| specific protein 65 kDa (RPE65) | | |
| Factor IX (FIX) | Hemophilia B | NP_000124.1 |
| Adenine nucleotide translocator | progressive external | NP_001142.2 |
| (ANT-I) | ophthalmoplegia | |
| ApaLI | mitochondrial heteroplasmy, | YP_007161330.1 |
| | myoclonic epilepsy with ragged | |
| | red fibers (MERRF) or | |
| | mitochondrial | |
| | encephalomyopathy, | |
| | lactic acidosis, and stroke-like | |
| | episodes (ME LAS) | |
| NADH ubiquinone | Leber hereditary optic | YP_003024035.1 |
| oxidoreductase subunit 4 (ND4) | | |
| very long-acyl-CoA | very long-chain acyl-CoA | NP_000009.1, |
| dehydrogenase (VLCAD) | dehydrogenase (VLCAD) | NP_001029031.1, |
| | deficiency | NP_001257376.1, |
| | | NP_001257377.1 |
| short-chain acyl-CoA | short-chain acyl-CoA | NP_000008.1 |
| dehydrogenase (SCAD) | dehydrogenase (SCAD) | |
| | deficiency | |
| medium-chain acyl-CoA | medium-chain acyl-CoA | NP_000007.1, |
| dehydrogenase (MCAD) | dehydrogenase (MCAD) | NP_001120800.1, |
| | deficiency | NP_001272971.1, |
| | | NP_001272972.1, |
| | | NP_001272973.1 |
| Myotubularin 1 (MTM1) | X-linked myotubular myopathy | NP_000243.1 |
| Myophosphorylase (PYGM) | McArdle disease (glycogen | NP_001158188.1, |
| | storage disease type V, | NP_005600.1 |
| | myophosphorylase deficiency) | |
| Lipoprotein lipase (LPL) | LPL deficiency | NP_000228.1 |
| sFLTOI (VEGF/PIGF (placental | Age-related macular | SEQ ID NO: 2, 8, 21, 23, or 25 |
| growth factor) binding domain of | degeneration | of W02009105669 |
| human VEGFRI/Flt-1 (hVEGFRI) | | |
| fused to the Fe portion of human | | |
| IgG(I) through a polyglycine | | |
| linker) | | |
| Glucocerebrosidase (GCase) | Gaucher disease | NP_000148.2, |
| | | NP_001005741.1, |
| | | NP_001005742.1, |
| | | NP_001165282.1, |
| | | NP_001165283.1 |
| Calsequestrin 2 (CASQ2) | Catecholaminergic polymorphic | NP_001223.2 |
| | ventricular tachycardia (CPVT) | |
| UDP glucuronosyltransferase 1 | Crigler-Najjar syndrome | NP_000454.1 |
| family member A1 (UGT1A1) | | |
| Glucose 6-phosphatase | GSD-Ia | NP_000142.2, |
| (G6Pase) | | NP_001257326.1 |
| Omithine carbamoyltransferase | OTC deficiency | NP_000522.3 |
| (OTC) | | |
| Cystathionine-β-synthase (CBS) | Homocystinuria | NP_000062.1, |
| | | NP_001171479.1, |
| | | NP_001171480.1 |
| Factor VIII (F8) | Haemophilia A | NP_000123.1, NP_063916.1 |
| Hemochromatosis (HFE) | Hemochromatosis | NP_000401.1, NP_620572.1, |
| | | NP_620573.1, NP_620575.1, |
| | | NP_620576.1, NP_620577.1, |
| | | NP_620578.1, NP_620579.1, |
| | | NP_620580.1 |
| Low density lipoprotein receptor | Phenylketonuria (PKU) | NP_000518.1, |
| (LDLR) | | NP_001182727.1, |
| | | NP_001182728.1, |
| | | NP_001182729.1, |
| | | NP_001182732.1 |
| Galactosidase, α (AGA) | Fabry disease | NP_000160.1 |
| Phenylalanine hydroxylase | Hypercholesterolaemia or | NP_000268.1 |
| (PAH) | Phenylketonuria (PKU) | |
| Propionyl CoA carboxylase, | Propionic acidaemias | NP_000273.2, |
| alpha polypeptide (PCCA) | | NP_001121164.1, |
| | | NP_001171475.1 |
| Brain-derived neurotrophic | Alzheimer's disease, | NP_001137281.1, |
| factor (BDNF) | Parkinson's disease, | NP_001137282.1, |
| | Huntington's disease, bipolar | NP_733930.1, |
| | disorder, anxiety, and eating | |
| | disorders | |
| Apolipoprotein E (ApoE) | Alzheimer's disease | NP_001289620.1, |
| | | NP_001289619.1, |
| | | NP_001289618.1, |
| | | NP_001289617.1, |
| | | NP_000032.1 |

Exemplary AAVs encoding transgenes encoding proteins described in Table 5 are useful for expressing or restoring healthy, physiological concentrations of said proteins in the CNS of healthy human subjects not having a neurocognitive or neuromuscular disorder. For example, the healthy, physiological concentration of GDNF is less than 10 ng/ml in the CNS. The healthy, physiological concentration of BDNF is less than or equal to 50 μg/mL in cerebrospinal fluid (CSF), and is up to approximately 25 μg/mL in the prefrontal cortex. The healthy, physiological concentration of ApoE is 4.5 ng/ml in CSF. The healthy, physiological concentration of GCase is 0.02-0.14 ng/ml in CSF.

The AAVs described herein may include a polynucleotide encoding a CNS protein associated with a lysosomal storage disorder. Exemplary proteins include, but are not limited to, a-galactosidase a, α-1-iduronidase, iduroate sulfatase, lysosomal acid α-glucosidase, sphingomyelinase, hexosaminidase A (HexA), hexosaminidase B (HexB), arylsulfatase A (ARSA), lysosomal acid lipase, acid ceramidase, galactosylceramidase, α-fucosidase, α-, β-mannosidosis, aspartylglucosaminidase, neuramidase, heparan-N-sulfatase, N-acetyl-α-glucosaminidase, Acetyl-CoA: α-glucosaminide N-acetyltransferase, N-acetylglucosamine-6-sulfate sulfatase, N-acetylgalactosamine-6-sulfate sulfatase, arylsulfatase B (ARSB), and B-glucuronidase. Exemplary AAVs encoding transgenes encoding proteins associated with lysosomal storage disorders are useful for expressing or restoring healthy, physiological concentrations of said proteins in the CNS of healthy human subjects not having a neurocognitive or neuromuscular disorder. For example, the healthy, physiological concentration of ARSA is 100 ng/mg in the CNS. The healthy, physiological concentration of HexA is 20 ng/ml in plasma, and the healthy, physiological concentration of HexB is 40 ng/mL in plasma.

### Routes of Administration

The AAVs described herein may be administered to a subject with FTD intrathalamically. In some embodiments, the AAV vector is administered to the patient in a convection-assisted manner.

In some embodiments, administration may include convection-assisted administration, for example, such as that described in Bobo et al. PNAS. 91:6 (1994): 2076-2080, the disclosure of which is incorporated herein by reference as it pertains to convection-assisted administration.

### Dosing Regimens

Using the compositions and methods of the disclosure, a patient having a neurocognitive or a neuromuscular disorder (e.g., a neurodegenerative disorder such as FTD, AD, PD, dementia with Lewy bodies, a related neurocognitive disorder, ALS, or a related motor neuron disorder), may be intrathalamically administered an AAV vector containing a transgene encoding PGRN (e.g., a codon-optimized PGRN) in an amount of from about 1×10⁹ vg/hemisphere to about 9×10¹² vg/hemisphere.

In some embodiments, the AAV vector is administered intrathalamically to the patient in an amount of from about 1×10⁹ vg/hemisphere to about 9×10¹² vg/hemisphere (e.g., 1×10⁹ vg/hemisphere to about 5×10¹² vg/hemisphere, 2×10⁹ vg/hemisphere to about 4×10¹² vg/hemisphere, 3×10⁹ vg/hemisphere to about 3×10¹² vg/hemisphere, 4×10⁹ vg/hemisphere to about 2×10¹² vg/hemisphere, 5×10⁹ vg/hemisphere to about 1×10¹² vg/hemisphere, 1×10¹⁰ vg/hemisphere to about 9×10¹¹ vg/hemisphere, 2×10¹⁰ vg/hemisphere to about 8×10¹¹ vg/hemisphere, 3×10¹⁰ vg/hemisphere to about 7×10¹¹ vg/hemisphere, 4×10¹⁰ vg/hemisphere to about 6×10¹¹ vg/hemisphere, 5×10¹⁰ vg/hemisphere to about 5×10¹¹ vg/hemisphere, or about 1×10¹¹ vg/hemisphere). For example, in some embodiments, the AAV vector is administered intrathalamically to the patient in an amount of from about 2×10⁹ vg/hemisphere to about 4×10¹² vg/hemisphere. In some embodiments, the AAV vector is administered intrathalamically to the patient in an amount of from about 3×10⁹ vg/hemisphere to about 3×10¹² vg/hemisphere. In some embodiments, the AAV vector is administered intrathalamically to the patient in an amount of from about 4×10⁹ vg/hemisphere to about 2×10¹² vg/hemisphere. In some embodiments, the AAV vector is administered intrathalamically to the patient in an amount of from about 5×10⁹ vg/hemisphere to about 1×10¹² vg/hemisphere. In some embodiments, the AAV vector is administered intrathalamically to the patient in an amount of from about 1×10¹⁰ vg/hemisphere to about 9×10¹¹ vg/hemisphere. In some embodiments, the AAV vector is administered intrathalamically to the patient in an amount of from about 2×10¹⁰ vg/hemisphere to about 8×10¹¹ vg/hemisphere. In some embodiments, the AAV vector is administered intrathalamically to the patient in an amount of from about 3×10¹⁰ vg/hemisphere to about 7×10¹¹ vg/hemisphere. In some embodiments, the AAV vector is administered intrathalamically to the patient in an amount of from about 4×10¹⁰ vg/hemisphere to about 6×10¹¹ vg/hemisphere. In some embodiments, the AAV vector is administered intrathalamically to the patient in an amount of from about 5×10¹⁰ vg/hemisphere to about 5×10¹¹ vg/hemisphere. In some embodiments, the AAV vector is administered intrathalamically to the patient in an amount of about 1×10¹¹ vg/hemisphere.

In some embodiments, the AAV vector is administered intrathalamically to the patient in an amount of from about 1×10¹⁰ vg/hemisphere to about 1×10¹² vg/hemisphere (e.g., 1×10¹⁰ vg/hemisphere to about 1×10¹² vg/hemisphere, 2×10¹⁰ vg/hemisphere to about 9×10¹¹ vg/hemisphere, 3×10¹⁰ vg/hemisphere to about 8×10¹¹ vg/hemisphere, 4×10¹⁰ vg/hemisphere to about 7×10¹¹ vg/hemisphere, 5×10¹⁰ vg/hemisphere to about 6×10¹¹ vg/hemisphere, 6×10¹⁰ vg/hemisphere to about 5×10¹¹ vg/hemisphere, 7×10¹⁰ vg/hemisphere to about 4×10¹¹ vg/hemisphere, 8×10¹⁰ vg/hemisphere to about 3×10¹¹ vg/hemisphere, 9×10¹⁰ vg/hemisphere to about 2×10¹¹ vg/hemisphere, or about 1×10¹¹ vg/hemisphere). For example, in some embodiments, the AAV vector is administered intrathalamically to the patient in an amount of from about 2×10¹⁰ vg/hemisphere to about 9×10¹¹ vg/hemisphere. In some embodiments, the AAV vector is administered intrathalamically to the patient in an amount of from about 3×10¹⁰ vg/hemisphere to about 8×10¹¹ vg/hemisphere. In some embodiments, the AAV vector is administered intrathalamically to the patient in an amount of from about 4×10¹⁰ vg/hemisphere to about 7×10¹¹ vg/hemisphere. In some embodiments, the AAV vector is administered intrathalamically to the patient in an amount of from about 5×10¹⁰ vg/hemisphere to about 6×10¹¹ vg/hemisphere. In some embodiments, the AAV vector is administered intrathalamically to the patient in an amount of from about 6×10¹⁰ vg/hemisphere to about 5×10¹¹ vg/hemisphere. In some embodiments, the AAV vector is administered intrathalamically to the patient in an amount of from about 7×10¹⁰ vg/hemisphere to about 4×10¹¹ vg/hemisphere. In some embodiments, the AAV vector is administered intrathalamically to the patient in an amount of from about 8×10¹⁰ vg/hemisphere to about 3×10¹¹ vg/hemisphere. In some embodiments, the AAV vector is administered intrathalamically to the patient in an amount of from about 9×10¹⁰ vg/hemisphere to about 2×10¹¹ vg/hemisphere. In some embodiments, the AAV vector is administered intrathalamically to the patient in an amount of about 1×10¹¹ vg/hemisphere.

In some embodiments, the AAV vector is administered intrathalamically to the patient in an amount of from about 5×10¹⁰ vg/hemisphere to about 1×10¹¹ vg/hemisphere (e.g., 5×10¹⁰ vg/hemisphere to about 1×10¹¹ vg/hemisphere, 6×10¹⁰ vg/hemisphere to about 9×10¹⁰ vg/hemisphere, or 7×10¹⁰ vg/hemisphere to about 8×10¹⁰ vg/hemisphere). For example, in some embodiment, the AAV vector is administered intrathalamically to the patient in an amount of from about 6×10¹⁰ vg/hemisphere to about 9×10¹⁰ vg/hemisphere. In some embodiments, the AAV vector is administered intrathalamically to the patient in an amount of from about 7×10¹⁰ vg/hemisphere to about 8×10¹⁰ vg/hemisphere.

In some embodiments, the AAV vector is administered intrathalamically to the patient in an amount of about 1×10⁹ vg/hemisphere. In some embodiments, the AAV vector is administered intrathalamically to the patient in an amount of about 2×10⁹ vg/hemisphere. In some embodiments, the AAV vector is administered intrathalamically to the patient in an amount of about 3×10⁹ vg/hemisphere. In some embodiments, the AAV vector is administered intrathalamically to the patient in an amount of about 4×10⁹ vg/hemisphere. In some embodiments, the AAV vector is administered intrathalamically to the patient in an amount of about 5×10⁹ vg/hemisphere. In some embodiments, the AAV vector is administered intrathalamically to the patient in an amount of about 6×10⁹ vg/hemisphere. In some embodiments, the AAV vector is administered intrathalamically to the patient in an amount of about 7×10⁹ vg/hemisphere. In some embodiments, the AAV vector is administered intrathalamically to the patient in an amount of about 8×10⁹ vg/hemisphere. In some embodiments, the AAV vector is administered intrathalamically to the patient in an amount of about 9×10⁹ vg/hemisphere. In some embodiments, the AAV vector is administered intrathalamically to the patient in an amount of about 1×10¹⁰ vg/hemisphere. In some embodiments, the AAV vector is administered intrathalamically to the patient in an amount of about 2×10¹⁰ vg/hemisphere. In some embodiments, the AAV vector is administered intrathalamically to the patient in an amount of about 3 ×10¹⁰ vg/hemisphere. In some embodiments, the AAV vector is administered intrathalamically to the patient in an amount of about 4×10¹⁰ vg/hemisphere. In some embodiments, the AAV vector is administered intrathalamically to the patient in an amount of about 5×10¹⁰ vg/hemisphere. In some embodiments, the AAV vector is administered intrathalamically to the patient in an amount of about 6×10¹⁰ vg/hemisphere. In some embodiments, the AAV vector is administered intrathalamically to the patient in an amount of about 7×10¹⁰ vg/hemisphere. In some embodiments, the AAV vector is administered intrathalamically to the patient in an amount of about 8×10¹⁰ vg/hemisphere. In some embodiments, the AAV vector is administered intrathalamically to the patient in an amount of about 9×10¹⁰ vg/hemisphere. In some embodiments, the AAV vector is administered intrathalamically to the patient in an amount of about 1×10¹¹ vg/hemisphere. In some embodiments, the AAV vector is administered intrathalamically to the patient in an amount of about 2×10¹¹ vg/hemisphere. In some embodiments, the AAV vector is administered intrathalamically to the patient in an amount of about 3×10¹¹ vg/hemisphere. In some embodiments, the AAV vector is administered intrathalamically to the patient in an amount of about 4×10¹¹ vg/hemisphere. In some embodiments, the AAV vector is administered intrathalamically to the patient in an amount of about 5×10¹¹ vg/hemisphere. In some embodiments, the AAV vector is administered intrathalamically to the patient in an amount of about 6×10¹¹ vg/hemisphere. In some embodiments, the AAV vector is administered intrathalamically to the patient in an amount of about 7×10¹¹ vg/hemisphere. In some embodiments, the AAV vector is administered intrathalamically to the patient in an amount of about 8×10¹¹ vg/hemisphere. In some embodiments, the AAV vector is administered intrathalamically to the patient in an amount of about 9×10¹¹ vg/hemisphere. In some embodiments, the AAV vector is administered intrathalamically to the patient in an amount of about 1×10¹² vg/hemisphere. In some embodiments, the AAV vector is administered intrathalamically to the patient in an amount of about 2×10¹² vg/hemisphere. In some embodiments, the AAV vector is administered intrathalamically to the patient in an amount of about 3×10¹² vg/hemisphere. In some embodiments, the AAV vector is administered intrathalamically to the patient in an amount of about 4×10¹² vg/hemisphere. In some embodiments, the AAV vector is administered intrathalamically to the patient in an amount of about 5×10¹² vg/hemisphere.

In some embodiments, the AAV vector comprising a transgene encoding PGRN is administered to the patient in an amount sufficient to achieve a level of PGRN expression in the frontal cortex of the patient that is equivalent to a level of PGRN expression observed in a human subject having a neurocognitive or a neuromuscular disorder (e.g., a neurodegenerative disorder such as FTD, AD, PD, dementia with Lewy bodies, a related neurocognitive disorder, ALS, or a related motor neuron disorder) following intrathalamic administration, in an amount of from about 1×10⁹ vg/hemisphere to about 9×10¹² vg/hemisphere (e.g., 1×10⁹ vg/hemisphere to about 5×10¹² vg/hemisphere, 2×10⁹ vg/hemisphere to about 4×10¹² vg/hemisphere, 3×10⁹ vg/hemisphere to about 3×10¹² vg/hemisphere, 4×10⁹ vg/hemisphere to about 2×10¹² vg/hemisphere, 5×10⁹ vg/hemisphere to about 1×10¹² vg/hemisphere, 1×10¹⁰ vg/hemisphere to about 9×10¹¹ vg/hemisphere, 2×10¹⁰ vg/hemisphere to about 8×10¹¹ vg/hemisphere, 3×10¹⁰ vg/hemisphere to about 7×10¹¹ vg/hemisphere, 4×10¹⁰ vg/hemisphere to about 6×10¹¹ vg/hemisphere, 5×10¹⁰ vg/hemisphere to about 5×10¹¹ vg/hemisphere, or about 1×10¹¹ vg/hemisphere), of an AAV2/9 vector having the nucleic acid sequence of SEQ ID NO: 6. For example, in some embodiments, the AAV vector comprising a transgene encoding PGRN is administered to the patient in an amount sufficient to achieve a level of PGRN expression in the frontal cortex of the patient that is equivalent to a level of PGRN expression observed in a human subject having a neurocognitive or a neuromuscular disorder (e.g., a neurodegenerative disorder such as FTD, AD, PD, dementia with Lewy bodies, a related neurocognitive disorder, ALS, or a related motor neuron disorder) following intrathalamic administration, in an amount of from about 2×10⁹ vg/hemisphere to about 4×10¹² vg/hemisphere, of an AAV2/9 vector having the nucleic acid sequence of SEQ ID NO: 6. In some embodiments the AAV vector comprising a transgene encoding PGRN is administered to the patient in an amount sufficient to achieve a level of PGRN expression in the frontal cortex of the patient that is equivalent to a level of PGRN expression observed in a human subject having a neurocognitive or a neuromuscular disorder (e.g., a neurodegenerative disorder such as FTD, AD, PD, dementia with Lewy bodies, a related neurocognitive disorder, ALS, or a related motor neuron disorder) following intrathalamic administration, in an amount of about 3×10⁹ vg/hemisphere to about 3×10¹² vg/hemisphere, of an AAV2/9 vector having the nucleic acid sequence of SEQ ID NO: 6. In some embodiments the AAV vector comprising a transgene encoding PGRN is administered to the patient in an amount sufficient to achieve a level of PGRN expression in the frontal cortex of the patient that is equivalent to a level of PGRN expression observed in a human subject having a neurocognitive or a neuromuscular disorder (e.g., a neurodegenerative disorder such as FTD, AD, PD, dementia with Lewy bodies, a related neurocognitive disorder, ALS, or a related motor neuron disorder) following intrathalamic administration, in an amount of about 4×10⁹ vg/hemisphere to about 2×10¹² vg/hemisphere, of an AAV2/9 vector having the nucleic acid sequence of SEQ ID NO: 6. In some embodiments, the AAV vector comprising a transgene encoding PGRN is administered to the patient in an amount sufficient to achieve a level of PGRN expression in the frontal cortex of the patient that is equivalent to a level of PGRN expression observed in a human subject having a neurocognitive or a neuromuscular disorder (e.g., a neurodegenerative disorder such as FTD, AD, PD, dementia with Lewy bodies, a related neurocognitive disorder, ALS, or a related motor neuron disorder) following intrathalamic administration, in an amount of about 5×10⁹ vg/hemisphere to about 1×10¹² vg/hemisphere, of an AAV2/9 vector having the nucleic acid sequence of SEQ ID NO: 6. In some embodiments, the AAV vector comprising a transgene encoding PGRN is administered to the patient in an amount sufficient to achieve a level of PGRN expression in the frontal cortex of the patient that is equivalent to a level of PGRN expression observed in a human subject having a neurocognitive or a neuromuscular disorder (e.g., a neurodegenerative disorder such as FTD, AD, PD, dementia with Lewy bodies, a related neurocognitive disorder, ALS, or a related motor neuron disorder) following intrathalamic administration, in an amount of about 1×10¹⁰ vg/hemisphere to about 9×10¹¹ vg/hemisphere, of an AAV2/9 vector having the nucleic acid sequence of SEQ ID NO: 6. In some embodiments, the AAV vector comprising a transgene encoding PGRN is administered to the patient in an amount sufficient to achieve a level of PGRN expression in the frontal cortex of the patient that is equivalent to a level of PGRN expression observed in a human subject having a neurocognitive or a neuromuscular disorder (e.g., a neurodegenerative disorder such as FTD, AD, PD, dementia with Lewy bodies, a related neurocognitive disorder, ALS, or a related motor neuron disorder) following intrathalamic administration, in an amount of about 2×10¹⁰ vg/hemisphere to about 8×10¹¹ vg/hemisphere, of an AAV2/9 vector having the nucleic acid sequence of SEQ ID NO: 6. In some embodiments the AAV vector comprising a transgene encoding PGRN is administered to the patient in an amount sufficient to achieve a level of PGRN expression in the frontal cortex of the patient that is equivalent to a level of PGRN expression observed in a human subject having a neurocognitive or a neuromuscular disorder (e.g., a neurodegenerative disorder such as FTD, AD, PD, dementia with Lewy bodies, a related neurocognitive disorder, ALS, or a related motor neuron disorder) following intrathalamic administration, in an amount of about 3×10¹⁰ vg/hemisphere to about 7×10¹¹ vg/hemisphere, of an AAV2/9 vector having the nucleic acid sequence of SEQ ID NO: 6. In some embodiments, the AAV vector comprising a transgene encoding PGRN is administered to the patient in an amount sufficient to achieve a level of PGRN expression in the frontal cortex of the patient that is equivalent to a level of PGRN expression observed in a human subject having a neurocognitive or a neuromuscular disorder (e.g., a neurodegenerative disorder such as FTD, AD, PD, dementia with Lewy bodies, a related neurocognitive disorder, ALS, or a related motor neuron disorder) following intrathalamic administration, in an amount of about 4×10¹⁰ vg/hemisphere to about 6×10¹¹ vg/hemisphere, of an AAV2/9 vector having the nucleic acid sequence of SEQ ID NO: 6. In some embodiments, the AAV vector comprising a transgene encoding PGRN is administered to the patient in an amount sufficient to achieve a level of PGRN expression in the frontal cortex of the patient that is equivalent to a level of PGRN expression observed in a human subject having a neurocognitive or a neuromuscular disorder (e.g., a neurodegenerative disorder such as FTD, AD, PD, dementia with Lewy bodies, a related neurocognitive disorder, ALS, or a related motor neuron disorder) following intrathalamic administration, in an amount of about 5×10¹⁰ vg/hemisphere to about 5×10¹¹ vg/hemisphere, of an AAV2/9 vector having the nucleic acid sequence of SEQ ID NO: 6. In some embodiments, the AAV vector comprising a transgene encoding PGRN is administered to the patient in an amount sufficient to achieve a level of PGRN expression in the frontal cortex of the patient that is equivalent to a level of PGRN expression observed in a human subject having a neurocognitive or a neuromuscular disorder (e.g., a neurodegenerative disorder such as FTD, AD, PD, dementia with Lewy bodies, a related neurocognitive disorder, ALS, or a related motor neuron disorder) following intrathalamic administration, in an amount of about 1×10¹¹ vg/hemisphere, of an AAV2/9 vector having the nucleic acid sequence of SEQ ID NO: 6.

In some embodiments, the AAV vector comprising a transgene encoding PGRN is administered to the patient in an amount sufficient to achieve a level of PGRN expression in the frontal cortex of the patient of from about 2 ng/mg to about 8 ng/mg (e.g., 3 ng/mg to about 7 ng/mg, 4 ng/mg to about 6 ng/mg, or about 5 ng/mg), or more (e.g., about 9 ng/mg, about 10 ng/mg, about 15 ng/mg, about 20 ng/mg, about 30 ng/mg, about 40 ng/mg, about 50 ng/mg, about 60 ng/mg, about 70 ng/mg, about 80 ng/mg, about 90 ng/mg, or about 100 ng/mg). For example, in some embodiments, the AAV vector comprising a transgene encoding PGRN is administered to the patient in an amount sufficient to achieve a level of PGRN expression in the frontal cortex of the patient of about 2 ng/mg in the frontal cortex. In some embodiments, the AAV vector comprising a transgene encoding PGRN is administered to the patient in an amount sufficient to achieve a level of PGRN expression in the frontal cortex of the patient of about 3 ng/mg in the frontal cortex. In some embodiments, the AAV vector comprising a transgene encoding PGRN is administered to the patient in an amount sufficient to achieve a level of PGRN expression in the frontal cortex of the patient of about 4 ng/mg in the frontal cortex. In some embodiments, the AAV vector comprising a transgene encoding PGRN is administered to the patient in an amount sufficient to achieve a level of PGRN expression in the frontal cortex of the patient of about 5 ng/mg in the frontal cortex. In some embodiments, the AAV vector comprising a transgene encoding PGRN is administered to the patient in an amount sufficient to achieve a level of PGRN expression in the frontal cortex of the patient of about 6 ng/mg in the frontal cortex. In some embodiments, the AAV vector comprising a transgene encoding PGRN is administered to the patient in an amount sufficient to achieve a level of PGRN expression in the frontal cortex of the patient of about 7 ng/mg in the frontal cortex. In some embodiments, the AAV vector comprising a transgene encoding PGRN is administered to the patient in an amount sufficient to achieve a level of PGRN expression in the frontal cortex of the patient of about 8 ng/mg in the frontal cortex. In some embodiments, the AAV vector comprising a transgene encoding PGRN is administered to the patient in an amount sufficient to achieve a level of PGRN expression in the frontal cortex of the patient of about 9 ng/mg in the frontal cortex. In some embodiments, the AAV vector comprising a transgene encoding PGRN is administered to the patient in an amount sufficient to achieve a level of PGRN expression in the frontal cortex of the patient of about 10 ng/mg in the frontal cortex. In some embodiments, the AAV vector comprising a transgene encoding PGRN is administered to the patient in an amount sufficient to achieve a level of PGRN expression in the frontal cortex of the patient of about 11 ng/mg in the frontal cortex. In some embodiments, the AAV vector comprising a transgene encoding PGRN is administered to the patient in an amount sufficient to achieve a level of PGRN expression in the frontal cortex of the patient of about 12 ng/mg in the frontal cortex. In some embodiments, the AAV vector comprising a transgene encoding PGRN is administered to the patient in an amount sufficient to achieve a level of PGRN expression in the frontal cortex of the patient of about 13 ng/mg in the frontal cortex. In some embodiments, the AAV vector comprising a transgene encoding PGRN is administered to the patient in an amount sufficient to achieve a level of PGRN expression in the frontal cortex of the patient of about 14 ng/mg in the frontal cortex. In some embodiments, the AAV vector comprising a transgene encoding PGRN is administered to the patient in an amount sufficient to achieve a level of PGRN expression in the frontal cortex of the patient of about 15 ng/mg in the frontal cortex. In some embodiments, the AAV vector comprising a transgene encoding PGRN is administered to the patient in an amount sufficient to achieve a level of PGRN expression in the frontal cortex of the patient of about 16 ng/mg in the frontal cortex. In some embodiments, the AAV vector comprising a transgene encoding PGRN is administered to the patient in an amount sufficient to achieve a level of PGRN expression in the frontal cortex of the patient of about 17 ng/mg in the frontal cortex. In some embodiments, the AAV vector comprising a transgene encoding PGRN is administered to the patient in an amount sufficient to achieve a level of PGRN expression in the frontal cortex of the patient of about 18 ng/mg in the frontal cortex. In some embodiments, the AAV vector comprising a transgene encoding PGRN is administered to the patient in an amount sufficient to achieve a level of PGRN expression in the frontal cortex of the patient of about 19 ng/mg in the frontal cortex. In some embodiments, the AAV vector comprising a transgene encoding PGRN is administered to the patient in an amount sufficient to achieve a level of PGRN expression in the frontal cortex of the patient of about 20 ng/mg in the frontal cortex. In some embodiments, the AAV vector comprising a transgene encoding PGRN is administered to the patient in an amount sufficient to achieve a level of PGRN expression in the frontal cortex of the patient of about 21 ng/mg in the frontal cortex. In some embodiments, the AAV vector comprising a transgene encoding PGRN is administered to the patient in an amount sufficient to achieve a level of PGRN expression in the frontal cortex of the patient of about 22 ng/mg in the frontal cortex. In some embodiments, the AAV vector comprising a transgene encoding PGRN is administered to the patient in an amount sufficient to achieve a level of PGRN expression in the frontal cortex of the patient of about 23 ng/mg in the frontal cortex. In some embodiments, the AAV vector comprising a transgene encoding PGRN is administered to the patient in an amount sufficient to achieve a level of PGRN expression in the frontal cortex of the patient of about 24 ng/mg in the frontal cortex. In some embodiments, the AAV vector comprising a transgene encoding PGRN is administered to the patient in an amount sufficient to achieve a level of PGRN expression in the frontal cortex of the patient of about 25 ng/mg in the frontal cortex. In some embodiments, the AAV vector comprising a transgene encoding PGRN is administered to the patient in an amount sufficient to achieve a level of PGRN expression in the frontal cortex of the patient of about 26 ng/mg in the frontal cortex. In some embodiments, the AAV vector comprising a transgene encoding PGRN is administered to the patient in an amount sufficient to achieve a level of PGRN expression in the frontal cortex of the patient of about 27 ng/mg in the frontal cortex. In some embodiments, the AAV vector comprising a transgene encoding PGRN is administered to the patient in an amount sufficient to achieve a level of PGRN expression in the frontal cortex of the patient of about 28 ng/mg in the frontal cortex. In some embodiments, the AAV vector comprising a transgene encoding PGRN is administered to the patient in an amount sufficient to achieve a level of PGRN expression in the frontal cortex of the patient of about 29 ng/mg in the frontal cortex. In some embodiments, the AAV vector comprising a transgene encoding PGRN is administered to the patient in an amount sufficient to achieve a level of PGRN expression in the frontal cortex of the patient of about 30 ng/mg in the frontal cortex. In some embodiments, the AAV vector comprising a transgene encoding PGRN is administered to the patient in an amount sufficient to achieve a level of PGRN expression in the frontal cortex of the patient of about 31 ng/mg in the frontal cortex. In some embodiments, the AAV vector comprising a transgene encoding PGRN is administered to the patient in an amount sufficient to achieve a level of PGRN expression in the frontal cortex of the patient of about 32 ng/mg in the frontal cortex. In some embodiments, the AAV vector comprising a transgene encoding PGRN is administered to the patient in an amount sufficient to achieve a level of PGRN expression in the frontal cortex of the patient of about 33 ng/mg in the frontal cortex. In some embodiments, the AAV vector comprising a transgene encoding PGRN is administered to the patient in an amount sufficient to achieve a level of PGRN expression in the frontal cortex of the patient of about 34 ng/mg in the frontal cortex. In some embodiments, the AAV vector comprising a transgene encoding PGRN is administered to the patient in an amount sufficient to achieve a level of PGRN expression in the frontal cortex of the patient of about 35 ng/mg in the frontal cortex. In some embodiments, the AAV vector comprising a transgene encoding PGRN is administered to the patient in an amount sufficient to achieve a level of PGRN expression in the frontal cortex of the patient of about 36 ng/mg in the frontal cortex. In some embodiments, the AAV vector comprising a transgene encoding PGRN is administered to the patient in an amount sufficient to achieve a level of PGRN expression in the frontal cortex of the patient of about 37 ng/mg in the frontal cortex. In some embodiments, the AAV vector comprising a transgene encoding PGRN is administered to the patient in an amount sufficient to achieve a level of PGRN expression in the frontal cortex of the patient of about 38 ng/mg in the frontal cortex. In some embodiments, the AAV vector comprising a transgene encoding PGRN is administered to the patient in an amount sufficient to achieve a level of PGRN expression in the frontal cortex of the patient of about 39 ng/mg in the frontal cortex. In some embodiments, the AAV vector comprising a transgene encoding PGRN is administered to the patient in an amount sufficient to achieve a level of PGRN expression in the frontal cortex of the patient of about 40 ng/mg in the frontal cortex. In some embodiments, the AAV vector comprising a transgene encoding PGRN is administered to the patient in an amount sufficient to achieve a level of PGRN expression in the frontal cortex of the patient of about 41 ng/mg in the frontal cortex. In some embodiments, the AAV vector comprising a transgene encoding PGRN is administered to the patient in an amount sufficient to achieve a level of PGRN expression in the frontal cortex of the patient of about 42 ng/mg in the frontal cortex. In some embodiments, the AAV vector comprising a transgene encoding PGRN is administered to the patient in an amount sufficient to achieve a level of PGRN expression in the frontal cortex of the patient of about 43 ng/mg in the frontal cortex. In some embodiments, the AAV vector comprising a transgene encoding PGRN is administered to the patient in an amount sufficient to achieve a level of PGRN expression in the frontal cortex of the patient of about 44 ng/mg in the frontal cortex. In some embodiments, the AAV vector comprising a transgene encoding PGRN is administered to the patient in an amount sufficient to achieve a level of PGRN expression in the frontal cortex of the patient of about 45 ng/mg in the frontal cortex. In some embodiments, the AAV vector comprising a transgene encoding PGRN is administered to the patient in an amount sufficient to achieve a level of PGRN expression in the frontal cortex of the patient of about 46 ng/mg in the frontal cortex. In some embodiments, the AAV vector comprising a transgene encoding PGRN is administered to the patient in an amount sufficient to achieve a level of PGRN expression in the frontal cortex of the patient of about 47 ng/mg in the frontal cortex. In some embodiments, the AAV vector comprising a transgene encoding PGRN is administered to the patient in an amount sufficient to achieve a level of PGRN expression in the frontal cortex of the patient of about 48 ng/mg in the frontal cortex. In some embodiments, the AAV vector comprising a transgene encoding PGRN is administered to the patient in an amount sufficient to achieve a level of PGRN expression in the frontal cortex of the patient of about 49 ng/mg in the frontal cortex. In some embodiments, the AAV vector comprising a transgene encoding PGRN is administered to the patient in an amount sufficient to achieve a level of PGRN expression in the frontal cortex of the patient of about 50 ng/mg in the frontal cortex. In some embodiments, the AAV vector comprising a transgene encoding PGRN is administered to the patient in an amount sufficient to achieve a level of PGRN expression in the frontal cortex of the patient of about 51 ng/mg in the frontal cortex. In some embodiments, the AAV vector comprising a transgene encoding PGRN is administered to the patient in an amount sufficient to achieve a level of PGRN expression in the frontal cortex of the patient of about 52 ng/mg in the frontal cortex. In some embodiments, the AAV vector comprising a transgene encoding PGRN is administered to the patient in an amount sufficient to achieve a level of PGRN expression in the frontal cortex of the patient of about 53 ng/mg in the frontal cortex. In some embodiments, the AAV vector comprising a transgene encoding PGRN is administered to the patient in an amount sufficient to achieve a level of PGRN expression in the frontal cortex of the patient of about 54 ng/mg in the frontal cortex. In some embodiments, the AAV vector comprising a transgene encoding PGRN is administered to the patient in an amount sufficient to achieve a level of PGRN expression in the frontal cortex of the patient of about 55 ng/mg in the frontal cortex. In some embodiments, the AAV vector comprising a transgene encoding PGRN is administered to the patient in an amount sufficient to achieve a level of PGRN expression in the frontal cortex of the patient of about 56 ng/mg in the frontal cortex. In some embodiments, the AAV vector comprising a transgene encoding PGRN is administered to the patient in an amount sufficient to achieve a level of PGRN expression in the frontal cortex of the patient of about 57 ng/mg in the frontal cortex. In some embodiments, the AAV vector comprising a transgene encoding PGRN is administered to the patient in an amount sufficient to achieve a level of PGRN expression in the frontal cortex of the patient of about 58 ng/mg in the frontal cortex. In some embodiments, the AAV vector comprising a transgene encoding PGRN is administered to the patient in an amount sufficient to achieve a level of PGRN expression in the frontal cortex of the patient of about 59 ng/mg in the frontal cortex. In some embodiments, the AAV vector comprising a transgene encoding PGRN is administered to the patient in an amount sufficient to achieve a level of PGRN expression in the frontal cortex of the patient of about 60 ng/mg in the frontal cortex. In some embodiments, the AAV vector comprising a transgene encoding PGRN is administered to the patient in an amount sufficient to achieve a level of PGRN expression in the frontal cortex of the patient of about 61 ng/mg in the frontal cortex. In some embodiments, the AAV vector comprising a transgene encoding PGRN is administered to the patient in an amount sufficient to achieve a level of PGRN expression in the frontal cortex of the patient of about 62 ng/mg in the frontal cortex. In some embodiments, the AAV vector comprising a transgene encoding PGRN is administered to the patient in an amount sufficient to achieve a level of PGRN expression in the frontal cortex of the patient of about 63 ng/mg in the frontal cortex. In some embodiments, the AAV vector comprising a transgene encoding PGRN is administered to the patient in an amount sufficient to achieve a level of PGRN expression in the frontal cortex of the patient of about 64 ng/mg in the frontal cortex. In some embodiments, the AAV vector comprising a transgene encoding PGRN is administered to the patient in an amount sufficient to achieve a level of PGRN expression in the frontal cortex of the patient of about 65 ng/mg in the frontal cortex. In some embodiments, the AAV vector comprising a transgene encoding PGRN is administered to the patient in an amount sufficient to achieve a level of PGRN expression in the frontal cortex of the patient of about 66 ng/mg in the frontal cortex. In some embodiments, the AAV vector comprising a transgene encoding PGRN is administered to the patient in an amount sufficient to achieve a level of PGRN expression in the frontal cortex of the patient of about 67 ng/mg in the frontal cortex. In some embodiments, the AAV vector comprising a transgene encoding PGRN is administered to the patient in an amount sufficient to achieve a level of PGRN expression in the frontal cortex of the patient of about 68 ng/mg in the frontal cortex. In some embodiments, the AAV vector comprising a transgene encoding PGRN is administered to the patient in an amount sufficient to achieve a level of PGRN expression in the frontal cortex of the patient of about 69 ng/mg in the frontal cortex. In some embodiments, the AAV vector comprising a transgene encoding PGRN is administered to the patient in an amount sufficient to achieve a level of PGRN expression in the frontal cortex of the patient of about 70 ng/mg in the frontal cortex. In some embodiments, the AAV vector comprising a transgene encoding PGRN is administered to the patient in an amount sufficient to achieve a level of PGRN expression in the frontal cortex of the patient of about 71 ng/mg in the frontal cortex. In some embodiments, the AAV vector comprising a transgene encoding PGRN is administered to the patient in an amount sufficient to achieve a level of PGRN expression in the frontal cortex of the patient of about 72 ng/mg in the frontal cortex. In some embodiments, the AAV vector comprising a transgene encoding PGRN is administered to the patient in an amount sufficient to achieve a level of PGRN expression in the frontal cortex of the patient of about 73 ng/mg in the frontal cortex. In some embodiments, the AAV vector comprising a transgene encoding PGRN is administered to the patient in an amount sufficient to achieve a level of PGRN expression in the frontal cortex of the patient of about 74 ng/mg in the frontal cortex. In some embodiments, the AAV vector comprising a transgene encoding PGRN is administered to the patient in an amount sufficient to achieve a level of PGRN expression in the frontal cortex of the patient of about 75 ng/mg in the frontal cortex. In some embodiments, the AAV vector comprising a transgene encoding PGRN is administered to the patient in an amount sufficient to achieve a level of PGRN expression in the frontal cortex of the patient of about 76 ng/mg in the frontal cortex. In some embodiments, the AAV vector comprising a transgene encoding PGRN is administered to the patient in an amount sufficient to achieve a level of PGRN expression in the frontal cortex of the patient of about 77 ng/mg in the frontal cortex. In some embodiments, the AAV vector comprising a transgene encoding PGRN is administered to the patient in an amount sufficient to achieve a level of PGRN expression in the frontal cortex of the patient of about 78 ng/mg in the frontal cortex. In some embodiments, the AAV vector comprising a transgene encoding PGRN is administered to the patient in an amount sufficient to achieve a level of PGRN expression in the frontal cortex of the patient of about 79 ng/mg in the frontal cortex. In some embodiments, the AAV vector comprising a transgene encoding PGRN is administered to the patient in an amount sufficient to achieve a level of PGRN expression in the frontal cortex of the patient of about 80 ng/mg in the frontal cortex. In some embodiments, the AAV vector comprising a transgene encoding PGRN is administered to the patient in an amount sufficient to achieve a level of PGRN expression in the frontal cortex of the patient of about 81 ng/mg in the frontal cortex. In some embodiments, the AAV vector comprising a transgene encoding PGRN is administered to the patient in an amount sufficient to achieve a level of PGRN expression in the frontal cortex of the patient of about 82 ng/mg in the frontal cortex. In some embodiments, the AAV vector comprising a transgene encoding PGRN is administered to the patient in an amount sufficient to achieve a level of PGRN expression in the frontal cortex of the patient of about 83 ng/mg in the frontal cortex. In some embodiments, the AAV vector comprising a transgene encoding PGRN is administered to the patient in an amount sufficient to achieve a level of PGRN expression in the frontal cortex of the patient of about 84 ng/mg in the frontal cortex. In some embodiments, the AAV vector comprising a transgene encoding PGRN is administered to the patient in an amount sufficient to achieve a level of PGRN expression in the frontal cortex of the patient of about 85 ng/mg in the frontal cortex. In some embodiments, the AAV vector comprising a transgene encoding PGRN is administered to the patient in an amount sufficient to achieve a level of PGRN expression in the frontal cortex of the patient of about 86 ng/mg in the frontal cortex. In some embodiments, the AAV vector comprising a transgene encoding PGRN is administered to the patient in an amount sufficient to achieve a level of PGRN expression in the frontal cortex of the patient of about 87 ng/mg in the frontal cortex. In some embodiments, the AAV vector comprising a transgene encoding PGRN is administered to the patient in an amount sufficient to achieve a level of PGRN expression in the frontal cortex of the patient of about 88 ng/mg in the frontal cortex. In some embodiments, the AAV vector comprising a transgene encoding PGRN is administered to the patient in an amount sufficient to achieve a level of PGRN expression in the frontal cortex of the patient of about 89 ng/mg in the frontal cortex. In some embodiments, the AAV vector comprising a transgene encoding PGRN is administered to the patient in an amount sufficient to achieve a level of PGRN expression in the frontal cortex of the patient of about 90 ng/mg in the frontal cortex. In some embodiments, the AAV vector comprising a transgene encoding PGRN is administered to the patient in an amount sufficient to achieve a level of PGRN expression in the frontal cortex of the patient of about 91 ng/mg in the frontal cortex. In some embodiments, the AAV vector comprising a transgene encoding PGRN is administered to the patient in an amount sufficient to achieve a level of PGRN expression in the frontal cortex of the patient of about 92 ng/mg in the frontal cortex. In some embodiments, the AAV vector comprising a transgene encoding PGRN is administered to the patient in an amount sufficient to achieve a level of PGRN expression in the frontal cortex of the patient of about 93 ng/mg in the frontal cortex. In some embodiments, the AAV vector comprising a transgene encoding PGRN is administered to the patient in an amount sufficient to achieve a level of PGRN expression in the frontal cortex of the patient of about 94 ng/mg in the frontal cortex. In some embodiments, the AAV vector comprising a transgene encoding PGRN is administered to the patient in an amount sufficient to achieve a level of PGRN expression in the frontal cortex of the patient of about 95 ng/mg in the frontal cortex. In some embodiments, the AAV vector comprising a transgene encoding PGRN is administered to the patient in an amount sufficient to achieve a level of PGRN expression in the frontal cortex of the patient of about 96 ng/mg in the frontal cortex. In some embodiments, the AAV vector comprising a transgene encoding PGRN is administered to the patient in an amount sufficient to achieve a level of PGRN expression in the frontal cortex of the patient of about 97 ng/mg in the frontal cortex. In some embodiments, the AAV vector comprising a transgene encoding PGRN is administered to the patient in an amount sufficient to achieve a level of PGRN expression in the frontal cortex of the patient of about 98 ng/mg in the frontal cortex. In some embodiments, the AAV vector comprising a transgene encoding PGRN is administered to the patient in an amount sufficient to achieve a level of PGRN expression in the frontal cortex of the patient of about 99 ng/mg in the frontal cortex. In some embodiments, the AAV vector comprising a transgene encoding PGRN is administered to the patient in an amount sufficient to achieve a level of PGRN expression in the frontal cortex of the patient of about 98 ng/mg in the frontal cortex. In some embodiments, the AAV vector comprising a transgene encoding PGRN is administered to the patient in an amount sufficient to achieve a level of PGRN expression in the frontal cortex of the patient of about 100 ng/mg in the frontal cortex.

In some embodiments, the AAV vector is administered to the patient in a single dose per hemisphere comprising the amount.

In some embodiments, the AAV vector is administered to the patient in a plurality (e.g., two, three, four, five, six, seven, eight, nine, or ten) of doses per hemisphere that, together, comprise the amount.

In some embodiments, the AAV vector is administered to the patient in two or more (e.g., two, three, four, five, six, seven, eight, nine, or ten) doses per hemisphere that each, individually, comprise the amount.

In some embodiments, the two or more (e.g., two, three, four, five, six, seven, eight, nine, or ten) doses per hemisphere are separated from one another by one year or more (e.g., one year, one year and one day, one year and one month, one year and six months, two years, three years, four years, or five years).

In some embodiments, the two or more (e.g., two, three, four, five, six, seven, eight, nine, or ten) doses per hemisphere are administered to the patient within about 12 months (e.g., about 12 months, about 11 months, about 10 months, about 9 months, about 8 months, about 7 months, about 6 months, about 5 months, about 4 months, about 3 months, about 2 months, or about 1 month) of one another.

In some embodiments, the AAV vector comprises a transgene encoding a protein that is associated with a neurocognitive disorder, neuromuscular disorder, or lysosomal storage disorder, such as a protein described in Table 5. In some embodiments, the AAV vector comprises a transgene encoding a protein that is associated with a lysosomal storage disorder, such as a-galactosidase a, α-1-iduronidase, iduroate sulfatase, lysosomal acid α-glucosidase, sphingomyelinase, hexosaminidase A, hexominidase B, arylsulfatase A, lysosomal acid lipase, acid ceramidase, galactosylceramidase, α-fucosidase, α-, β-mannosidosis, aspartylglucosaminidase, neuramidase, heparan-N-sulfatase, N-acetyl-α-glucosaminidase, Acetyl-CoA: α-glucosaminide N-acetyltransferase, N-acetylglucosamine-6-sulfate sulfatase, N-acetylgalactosamine-6-sulfate sulfatase, arylsulfatase A, arylsulfatase B, and B-glucuronidase.

The AAV described herein can be administered in an amount sufficient to improve one or more pathological features in a neurocognitive or a neuromuscular disorder (e.g., a neurodegenerative disorder such as FTD, AD, PD, dementia with Lewy bodies, a related neurocognitive disorder, ALS, or a related motor neuron disorder). Administration of the AAV described herein may improve the cognitive performance of the subject, restore expression of the protein encoded by the transgene in the frontal cortex (e.g., from about 2 ng/mg to about 8 ng/mg (e.g., 3 ng/mg to about 7 ng/mg, 4 ng/mg to about 6 ng/mg, or about 5 ng/mg) or more (e.g., about 9 ng/mg, about 10 ng/mg, about 15 ng/mg, about 20 ng/mg, about 30 ng/mg, about 40 ng/mg, about 50 ng/mg, about 60 ng/mg, about 70 ng/mg, about 80 ng/mg, about 90 ng/mg, or about 100 ng/mg), improve the motor function of the subject, reduce α-synuclein protein levels, tau-positive neuronal inclusion levels, and/or TAR DNA-binding protein 43 (TDP-43)-positive inclusion levels in the brain tissue in the subject. Cognition and motor function can be assessed using standard neurological tests before and after treatment, and protein levels (e.g., PGRN) can be detected in plasma and cerebrospinal fluid (CSF) using ELISA. Neurodegeneration can be assessed using F18-fluorodeoxyglucose PET scans or MRI scans. The patient may be evaluated 1 month, 2 months, 3 months, 4 months, 5 months, 6 months or more following administration of the AAV. Depending on the outcome of the evaluation, the patient may receive additional treatments.

### Pharmaceutical Compositions

The AAVs described herein can be formulated into pharmaceutical compositions for administration to a patient, such as a human patient exhibiting or at risk of a neurocognitive or a neuromuscular disorder (e.g., a neurodegenerative disorder such as FTD, AD, PD, dementia with Lewy bodies, a related neurocognitive disorder, ALS, or a related motor neuron disorder), in a biologically compatible form suitable for administration in vivo. A pharmaceutical composition containing, for example, an AAV including one or more transgenes encoding PGRN described herein, typically includes a pharmaceutically acceptable diluent or carrier. A pharmaceutical composition may include (e.g., consist of), e.g., a sterile saline solution and a nucleic acid. The sterile saline is typically a pharmaceutical grade saline. A pharmaceutical composition may include (e.g., consist of), e.g., sterile water and a nucleic acid. The sterile water is typically a pharmaceutical grade water. A pharmaceutical composition may include (e.g., consist of), e.g., phosphate-buffered saline (PBS) and a nucleic acid. The sterile PBS is typically a pharmaceutical grade PBS.

In certain embodiments, pharmaceutical compositions include one or more composition or nucleic acid molecule and one or more excipients. In certain embodiments, excipients are selected from water, salt solutions, alcohol, polyethylene glycols, gelatin, lactose, amylase, magnesium stearate, talc, silicic acid, viscous paraffin, hydroxymethylcellulose and polyvinylpyrrolidone.

In certain embodiments, nucleic acid molecules may be admixed with pharmaceutically acceptable active and/or inert substances for the preparation of pharmaceutical compositions or formulations. Compositions and methods for the formulation of pharmaceutical compositions depend on a number of criteria, including, but not limited to, route of administration, extent of disease, or dose to be administered.

In certain embodiments, pharmaceutical compositions including a nucleic acid molecule encompass any pharmaceutically acceptable salts of the inhibitor, esters of the inhibitor, or salts of such esters. In certain embodiments, pharmaceutical compositions including a nucleic acid molecule, upon administration to a subject (e.g., a human), are capable of providing (directly or indirectly) the biologically active metabolite or residue thereof. Accordingly, for example, the disclosure is also drawn to pharmaceutically acceptable salts of inhibitors, prodrugs, pharmaceutically acceptable salts of such prodrugs, and other bioequivalents. Suitable pharmaceutically acceptable salts include, but are not limited to, sodium and potassium salts. In certain embodiments, prodrugs include one or more conjugate group attached to a nucleic acid molecule, wherein the conjugate group is cleaved by endogenous nucleases within the body.

Lipid moieties have been used in nucleic acid therapies in a variety of methods. In certain such methods, the nucleic acid is introduced into preformed liposomes or lipoplexes made of mixtures of cationic lipids and neutral lipids. In certain methods, DNA complexes with mono- or poly-cationic lipids are formed without the presence of a neutral lipid. In certain embodiments, a lipid moiety is selected to increase distribution of a pharmaceutical agent to a particular cell or tissue. In certain embodiments, a lipid moiety is selected to increase distribution of a pharmaceutical agent to fat tissue. In certain embodiments, a lipid moiety is selected to increase distribution of a pharmaceutical agent to muscle tissue.

In certain embodiments, pharmaceutical compositions include a delivery system. Examples of delivery systems include, but are not limited to, liposomes and emulsions. Certain delivery systems are useful for preparing certain pharmaceutical compositions including those including hydrophobic compounds. In certain embodiments, certain organic solvents such as dimethylsulfoxide are used.

In certain embodiments, pharmaceutical compositions include one or more tissue-specific delivery molecules designed to deliver the one or more pharmaceutical agents of the present invention to specific tissues or cell types. For example, in certain embodiments, pharmaceutical compositions include liposomes coated with a tissue-specific antibody.

In certain embodiments, pharmaceutical compositions include a co-solvent system. Certain of such co-solvent systems include, for example, benzyl alcohol, a nonpolar surfactant, a water-miscible organic polymer, and an aqueous phase. In certain embodiments, such co-solvent systems are used for hydrophobic compounds. A non-limiting example of such a co-solvent system is the VPD co-solvent system, which is a solution of absolute ethanol including 3% w/v benzyl alcohol, 8% w/v of the nonpolar surfactant Polysorbate 80™ and 65% w/v polyethylene glycol 300. The proportions of such co-solvent systems may be varied considerably without significantly altering their solubility and toxicity characteristics. Furthermore, the identity of co-solvent components may be varied: for example, other surfactants may be used instead of Polysorbate 80™; the fraction size of polyethylene glycol may be varied; other biocompatible polymers may replace polyethylene glycol, e.g., polyvinyl pyrrolidone; and other sugars or polysaccharides may substitute for dextrose.

In certain embodiments, pharmaceutical compositions are prepared for intrathalamic administration. In such embodiments, a pharmaceutical composition may include a carrier and is formulated in aqueous solution, such as water or physiologically compatible buffers such as Hanks's solution, Ringer's solution, or physiological saline buffer. In some embodiments, other ingredients are included (e.g., ingredients that aid in solubility or serve as preservatives). In some embodiments, injectable suspensions are prepared using appropriate liquid carriers, suspending agents and the like. Certain pharmaceutical compositions for injection are presented in unit dosage form, e.g., in ampoules or in multi-dose containers. Certain pharmaceutical compositions for injection are suspensions, solutions or emulsions in oily or aqueous vehicles, and may contain formulatory agents such as suspending, stabilizing and/or dispersing agents. Certain solvents suitable for use in pharmaceutical compositions for injection include, but are not limited to, lipophilic solvents and fatty oils, such as sesame oil, synthetic fatty acid esters, such as ethyl oleate or triglycerides, and liposomes.

### Monitoring Efficacy

Clinical efficacy can be monitored using biomarkers among other methods. Measurable biomarkers to monitor efficacy include, but are not limited to, monitoring one or more of the physical symptoms of a neurocognitive or a neuromuscular disorder (e.g., a neurodegenerative disorder such as FTD, AD, PD, dementia with Lewy bodies, a related neurocognitive disorder, ALS, or a related motor neuron disorder). These may include tremors, muscle spasms or weakness (e.g., affecting an arm, a leg, neck or diaphragm), rigidity (e.g., rigid muscles), poor coordination and/or balance, difficulty chewing or swallowing, weight gain due to dramatic overeating, stiff muscles, feet that shuffle or drag upon walking, trouble standing or sitting up in a chair, fatigue, trouble controlling the bladder, seizures, uncontrollable twitching (e.g., fasciculations in the arm, leg, shoulder, or tongue), bradykinesia, impaired posture, loss of automatic movements, speech changes, writing changes, poor regulation of body functions (e.g., autonomic), difficulty sleeping, clumsiness, stumbling, slurred speech, or muscle wasting. Observation of the stabilization, improvement and/or reversal of one or more symptoms indicates that the treatment or prevention regime is efficacious. Observation of the progression, increase or exacerbation of one or more symptoms indicates that the treatment or prevention regime is not efficacious. A preferred biomarker for assessing treatment in of a neurocognitive or a neuromuscular disorder (e.g., a neurodegenerative disorder such as FTD, AD, PD, dementia with Lewy bodies, a related neurocognitive disorder, ALS, or a related motor neuron disorder) is a level of PGRN. This marker is preferably assessed at the protein level, but measurement of mRNA encoding PGRN can also be used as a surrogate measure of PGRN expression. Such a level can be measured in a blood sample. Such a level is reduced in subjects with a neurocognitive or a neuromuscular disorder (e.g., a neurodegenerative disorder such as FTD, AD, PD, dementia with Lewy bodies, a related neurocognitive disorder, ALS, or a related motor neuron disorder) relative to a control population of undiseased individuals (e.g., the patient exhibits a level of expression of endogenous PGRN that is from about 1% to about 40% of the level of endogenous PGRN expression observed in a human subject of the same age, gender, and/or body mass index that does not have a neurocognitive or a neuromuscular disorder (e.g., a neurodegenerative disorder such as FTD, AD, PD, dementia with Lewy bodies, a related neurocognitive disorder, ALS, or a related motor neuron disorder). Therefore, an increase in level provides an indication of a favorable treatment response, whereas an unchanged or decreasing levels provides an indication of unfavorable or at least non-optimal treatment response.

In certain embodiments, the monitoring methods can entail determining a baseline value of a measurable biomarker or disease parameter in a subject before administering a dosage of the AAV described herein and comparing this with a value for the same measurable biomarker or parameter after a course of treatment.

In other methods, a control value (i.e., a mean and standard deviation) of the measurable biomarker or parameter is determined for a control population. For example, in some embodiments, prior to administration of the AAV vector, the patient exhibits a level of expression of endogenous PGRN that is from about 1% to about 40% of the level of endogenous PGRN expression observed in a human subject of the same age, gender, and/or body mass index that does not have a neurocognitive or a neuromuscular disorder (e.g., a neurodegenerative disorder such as FTD, AD, PD, dementia with Lewy bodies, a related neurocognitive disorder, ALS, or a related motor neuron disorder). In certain embodiments, the individuals in the control population have not received prior treatment and do not have a neurocognitive or a neuromuscular disorder (e.g., a neurodegenerative disorder such as FTD, AD, PD, dementia with Lewy bodies, a related neurocognitive disorder, ALS, or a related motor neuron disorder), nor are at risk of developing a neurocognitive or a neuromuscular disorder (e.g., a neurodegenerative disorder such as FTD, AD, PD, dementia with Lewy bodies, a related neurocognitive disorder, ALS, or a related motor neuron disorder). In such cases, if the value of the measurable biomarker or clinical parameter approaches the control value, then treatment is considered efficacious. In other embodiments, the individuals in the control population have not received prior treatment and have been diagnosed with a neurocognitive or a neuromuscular disorder (e.g., a neurodegenerative disorder such as FTD, AD, PD, dementia with Lewy bodies, a related neurocognitive disorder, ALS, or a related motor neuron disorder). In such cases, if the value of the measurable biomarker or clinical parameter approaches the control value, then treatment is considered inefficacious.

In other methods, a subject who is not presently receiving treatment but has undergone a previous course of treatment is monitored for one or more of the biomarkers or clinical parameters to determine whether a resumption of treatment is required. The measured value of one or more of the biomarkers or clinical parameters in the subject can be compared with a value previously achieved in the subject after a previous course of treatment. Alternatively, the value measured in the subject can be compared with a control value (mean plus standard deviation) determined in population of subjects after undergoing a course of treatment. Alternatively, the measured value in the subject can be compared with a control value in populations of prophylactically treated subjects who remain free of symptoms of disease, or populations of therapeutically treated subjects who show amelioration of disease characteristics. In such cases, if the value of the measurable biomarker or clinical parameter approaches the control value, then treatment is considered efficacious and need not be resumed. In all of these cases, a significant difference relative to the control level (i.e., more than a standard deviation) is an indicator that treatment should be resumed in the subject.

In some embodiments, following administration of the AAV vector, the patient exhibits an increase in PGRN expression relative to a measurement of the patient's PGRN expression level obtained prior to administration of the AAV vector. In some embodiments, the increase in PGRN expression is observed in the patient's thalamus, frontal cortex, basal ganglia, parietal cortex, temporal cortex, parietal and temporal cortices, and/or CSF.

In some embodiments, following administration of the AAV vector, the patient exhibits a level of PGRN expression of from about 2 ng/mg to about 100 ng/mg (e.g., 3 ng/mg to about 99 ng/mg, 4 ng/mg to about 98 ng/mg, 5 ng/mg to about 97 ng/mg, 10 ng/mg to about 90 ng/mg, 20 ng/mg to about 80 ng/mg, 30 ng/mg to about 70 ng/mg, 40 ng/mg to about 60 ng/mg, or about 50 ng/mg) in the frontal cortex. For example, in some embodiments, following administration of the AAV vector, the patient exhibits a level of PGRN expression of from about 3 ng/mg to about 99 ng/mg in the frontal cortex. In some embodiments, following administration of the AAV vector, the patient exhibits a level of PGRN expression of from about 4 ng/mg to about 98 ng/mg in the frontal cortex. In some embodiments, following administration of the AAV vector, the patient exhibits a level of PGRN expression of from about 5 ng/mg to about 97 ng/mg in the frontal cortex. In some embodiments, following administration of the AAV vector, the patient exhibits a level of PGRN expression of from about 10 ng/mg to about 90 ng/mg in the frontal cortex. In some embodiments, following administration of the AAV vector, the patient exhibits a level of PGRN expression of from about 20 ng/mg to about 80 ng/mg. In some embodiments, following administration of the AAV vector, the patient exhibits a level of PGRN expression of from about 30 ng/mg to about 70 ng/mg. In some embodiments, following administration of the AAV vector, the patient exhibits a level of PGRN expression of from about 40 ng/mg to about 60 ng/mg. In some embodiments, following administration of the AAV vector, the patient exhibits a level of PGRN expression of about 50 ng/mg.

In some embodiments, following administration of the AAV vector, the patient exhibits a level of PGRN expression of about 2 ng/mg in the frontal cortex. In some embodiments, following administration of the AAV vector, the patient exhibits a level of PGRN expression of about 3 ng/mg in the frontal cortex. In some embodiments, following administration of the AAV vector, the patient exhibits a level of PGRN expression of about 4 ng/mg in the frontal cortex. In some embodiments, following administration of the AAV vector, the patient exhibits a level of PGRN expression of about 5 ng/mg in the frontal cortex. In some embodiments, following administration of the AAV vector, the patient exhibits a level of PGRN expression of about 6 ng/mg in the frontal cortex. In some embodiments, following administration of the AAV vector, the patient exhibits a level of PGRN expression of about 7 ng/mg in the frontal cortex. In some embodiments, following administration of the AAV vector, the patient exhibits a level of PGRN expression of about 8 ng/mg in the frontal cortex. In some embodiments, following administration of the AAV vector, the patient exhibits a level of PGRN expression of about 9 ng/mg in the frontal cortex. In some embodiments, following administration of the AAV vector, the patient exhibits a level of PGRN expression of about 10 ng/mg in the frontal cortex. In some embodiments, following administration of the AAV vector, the patient exhibits a level of PGRN expression of about 11 ng/mg in the frontal cortex. In some embodiments, following administration of the AAV vector, the patient exhibits a level of PGRN expression of about 12 ng/mg in the frontal cortex. In some embodiments, following administration of the AAV vector, the patient exhibits a level of PGRN expression of about 13 ng/mg in the frontal cortex. In some embodiments, following administration of the AAV vector, the patient exhibits a level of PGRN expression of about 14 ng/mg in the frontal cortex. In some embodiments, following administration of the AAV vector, the patient exhibits a level of PGRN expression of about 15 ng/mg in the frontal cortex. In some embodiments, following administration of the AAV vector, the patient exhibits a level of PGRN expression of about 16 ng/mg in the frontal cortex. In some embodiments, following administration of the AAV vector, the patient exhibits a level of PGRN expression of about 17 ng/mg in the frontal cortex. In some embodiments, following administration of the AAV vector, the patient exhibits a level of PGRN expression of about 18 ng/mg in the frontal cortex. In some embodiments, following administration of the AAV vector, the patient exhibits a level of PGRN expression of about 19 ng/mg in the frontal cortex. In some embodiments, following administration of the AAV vector, the patient exhibits a level of PGRN expression of about 20 ng/mg in the frontal cortex. In some embodiments, following administration of the AAV vector, the patient exhibits a level of PGRN expression of about 21 ng/mg in the frontal cortex. In some embodiments, following administration of the AAV vector, the patient exhibits a level of PGRN expression of about 22 ng/mg in the frontal cortex. In some embodiments, following administration of the AAV vector, the patient exhibits a level of PGRN expression of about 23 ng/mg in the frontal cortex. In some embodiments, following administration of the AAV vector, the patient exhibits a level of PGRN expression of about 24 ng/mg in the frontal cortex. In some embodiments, following administration of the AAV vector, the patient exhibits a level of PGRN expression of about 25 ng/mg in the frontal cortex. In some embodiments, following administration of the AAV vector, the patient exhibits a level of PGRN expression of about 26 ng/mg in the frontal cortex. In some embodiments, following administration of the AAV vector, the patient exhibits a level of PGRN expression of about 27 ng/mg in the frontal cortex. In some embodiments, following administration of the AAV vector, the patient exhibits a level of PGRN expression of about 28 ng/mg in the frontal cortex. In some embodiments, following administration of the AAV vector, the patient exhibits a level of PGRN expression of about 29 ng/mg in the frontal cortex. In some embodiments, following administration of the AAV vector, the patient exhibits a level of PGRN expression of about 30 ng/mg in the frontal cortex. In some embodiments, following administration of the AAV vector, the patient exhibits a level of PGRN expression of about 31 ng/mg in the frontal cortex. In some embodiments, following administration of the AAV vector, the patient exhibits a level of PGRN expression of about 32 ng/mg in the frontal cortex. In some embodiments, following administration of the AAV vector, the patient exhibits a level of PGRN expression of about 33 ng/mg in the frontal cortex. In some embodiments, following administration of the AAV vector, the patient exhibits a level of PGRN expression of about 34 ng/mg in the frontal cortex. In some embodiments, following administration of the AAV vector, the patient exhibits a level of PGRN expression of about 35 ng/mg in the frontal cortex. In some embodiments, following administration of the AAV vector, the patient exhibits a level of PGRN expression of about 36 ng/mg in the frontal cortex. In some embodiments, following administration of the AAV vector, the patient exhibits a level of PGRN expression of about 37 ng/mg in the frontal cortex. In some embodiments, following administration of the AAV vector, the patient exhibits a level of PGRN expression of about 38 ng/mg in the frontal cortex. In some embodiments, following administration of the AAV vector, the patient exhibits a level of PGRN expression of about 39 ng/mg in the frontal cortex. In some embodiments, following administration of the AAV vector, the patient exhibits a level of PGRN expression of about 40 ng/mg in the frontal cortex. In some embodiments, following administration of the AAV vector, the patient exhibits a level of PGRN expression of about 41 ng/mg in the frontal cortex. In some embodiments, following administration of the AAV vector, the patient exhibits a level of PGRN expression of about 42 ng/mg in the frontal cortex. In some embodiments, following administration of the AAV vector, the patient exhibits a level of PGRN expression of about 43 ng/mg in the frontal cortex. In some embodiments, following administration of the AAV vector, the patient exhibits a level of PGRN expression of about 44 ng/mg in the frontal cortex. In some embodiments, following administration of the AAV vector, the patient exhibits a level of PGRN expression of about 45 ng/mg in the frontal cortex. In some embodiments, following administration of the AAV vector, the patient exhibits a level of PGRN expression of about 46 ng/mg in the frontal cortex. In some embodiments, following administration of the AAV vector, the patient exhibits a level of PGRN expression of about 47 ng/mg in the frontal cortex. In some embodiments, following administration of the AAV vector, the patient exhibits a level of PGRN expression of about 48 ng/mg in the frontal cortex. In some embodiments, following administration of the AAV vector, the patient exhibits a level of PGRN expression of about 49 ng/mg in the frontal cortex. In some embodiments, following administration of the AAV vector, the patient exhibits a level of PGRN expression of about 50 ng/mg in the frontal cortex. In some embodiments, following administration of the AAV vector, the patient exhibits a level of PGRN expression of about 51 ng/mg in the frontal cortex. In some embodiments, following administration of the AAV vector, the patient exhibits a level of PGRN expression of about 52 ng/mg in the frontal cortex. In some embodiments, following administration of the AAV vector, the patient exhibits a level of PGRN expression of about 53 ng/mg in the frontal cortex. In some embodiments, following administration of the AAV vector, the patient exhibits a level of PGRN expression of about 54 ng/mg in the frontal cortex. In some embodiments, following administration of the AAV vector, the patient exhibits a level of PGRN expression of about 55 ng/mg in the frontal cortex. In some embodiments, following administration of the AAV vector, the patient exhibits a level of PGRN expression of about 56 ng/mg in the frontal cortex. In some embodiments, following administration of the AAV vector, the patient exhibits a level of PGRN expression of about 57 ng/mg in the frontal cortex. In some embodiments, following administration of the AAV vector, the patient exhibits a level of PGRN expression of about 58 ng/mg in the frontal cortex. In some embodiments, following administration of the AAV vector, the patient exhibits a level of PGRN expression of about 59 ng/mg in the frontal cortex. In some embodiments, following administration of the AAV vector, the patient exhibits a level of PGRN expression of about 60 ng/mg in the frontal cortex. In some embodiments, following administration of the AAV vector, the patient exhibits a level of PGRN expression of about 61 ng/mg in the frontal cortex. In some embodiments, following administration of the AAV vector, the patient exhibits a level of PGRN expression of about 62 ng/mg in the frontal cortex. In some embodiments, following administration of the AAV vector, the patient exhibits a level of PGRN expression of about 63 ng/mg in the frontal cortex. In some embodiments, following administration of the AAV vector, the patient exhibits a level of PGRN expression of about 64 ng/mg in the frontal cortex. In some embodiments, following administration of the AAV vector, the patient exhibits a level of PGRN expression of about 65 ng/mg in the frontal cortex. In some embodiments, following administration of the AAV vector, the patient exhibits a level of PGRN expression of about 66 ng/mg in the frontal cortex. In some embodiments, following administration of the AAV vector, the patient exhibits a level of PGRN expression of about 67 ng/mg in the frontal cortex. In some embodiments, following administration of the AAV vector, the patient exhibits a level of PGRN expression of about 68 ng/mg in the frontal cortex. In some embodiments, following administration of the AAV vector, the patient exhibits a level of PGRN expression of about 69 ng/mg in the frontal cortex. In some embodiments, following administration of the AAV vector, the patient exhibits a level of PGRN expression of about 70 ng/mg in the frontal cortex. In some embodiments, following administration of the AAV vector, the patient exhibits a level of PGRN expression of about 71 ng/mg in the frontal cortex. In some embodiments, following administration of the AAV vector, the patient exhibits a level of PGRN expression of about 72 ng/mg in the frontal cortex. In some embodiments, following administration of the AAV vector, the patient exhibits a level of PGRN expression of about 73 ng/mg in the frontal cortex. In some embodiments, following administration of the AAV vector, the patient exhibits a level of PGRN expression of about 74 ng/mg in the frontal cortex. In some embodiments, following administration of the AAV vector, the patient exhibits a level of PGRN expression of about 75 ng/mg in the frontal cortex. In some embodiments, following administration of the AAV vector, the patient exhibits a level of PGRN expression of about 76 ng/mg in the frontal cortex. In some embodiments, following administration of the AAV vector, the patient exhibits a level of PGRN expression of about 77 ng/mg in the frontal cortex. In some embodiments, following administration of the AAV vector, the patient exhibits a level of PGRN expression of about 78 ng/mg in the frontal cortex. In some embodiments, following administration of the AAV vector, the patient exhibits a level of PGRN expression of about 79 ng/mg in the frontal cortex. In some embodiments, following administration of the AAV vector, the patient exhibits a level of PGRN expression of about 80 ng/mg in the frontal cortex. In some embodiments, following administration of the AAV vector, the patient exhibits a level of PGRN expression of about 81 ng/mg in the frontal cortex. In some embodiments, following administration of the AAV vector, the patient exhibits a level of PGRN expression of about 82 ng/mg in the frontal cortex. In some embodiments, following administration of the AAV vector, the patient exhibits a level of PGRN expression of about 83 ng/mg in the frontal cortex. In some embodiments, following administration of the AAV vector, the patient exhibits a level of PGRN expression of about 84 ng/mg in the frontal cortex. In some embodiments, following administration of the AAV vector, the patient exhibits a level of PGRN expression of about 85 ng/mg in the frontal cortex. In some embodiments, following administration of the AAV vector, the patient exhibits a level of PGRN expression of about 86 ng/mg in the frontal cortex. In some embodiments, following administration of the AAV vector, the patient exhibits a level of PGRN expression of about 87 ng/mg in the frontal cortex. In some embodiments, following administration of the AAV vector, the patient exhibits a level of PGRN expression of about 88 ng/mg in the frontal cortex. In some embodiments, following administration of the AAV vector, the patient exhibits a level of PGRN expression of about 89 ng/mg in the frontal cortex. In some embodiments, following administration of the AAV vector, the patient exhibits a level of PGRN expression of about 90 ng/mg in the frontal cortex. In some embodiments, following administration of the AAV vector, the patient exhibits a level of PGRN expression of about 91 ng/mg in the frontal cortex. In some embodiments, following administration of the AAV vector, the patient exhibits a level of PGRN expression of about 92 ng/mg in the frontal cortex. In some embodiments, following administration of the AAV vector, the patient exhibits a level of PGRN expression of about 93 ng/mg in the frontal cortex. In some embodiments, following administration of the AAV vector, the patient exhibits a level of PGRN expression of about 94 ng/mg in the frontal cortex. In some embodiments, following administration of the AAV vector, the patient exhibits a level of PGRN expression of about 95 ng/mg in the frontal cortex. In some embodiments, following administration of the AAV vector, the patient exhibits a level of PGRN expression of about 96 ng/mg in the frontal cortex. In some embodiments, following administration of the AAV vector, the patient exhibits a level of PGRN expression of about 97 ng/mg in the frontal cortex. In some embodiments, following administration of the AAV vector, the patient exhibits a level of PGRN expression of about 98 ng/mg in the frontal cortex. In some embodiments, following administration of the AAV vector, the patient exhibits a level of PGRN expression of about 99 ng/mg in the frontal cortex. In some embodiments, following administration of the AAV vector, the patient exhibits a level of PGRN expression of about 98 ng/mg in the frontal cortex. In some embodiments, following administration of the AAV vector, the patient exhibits a level of PGRN expression of about 100 ng/mg in the frontal cortex.

In some embodiments, following administration of the AAV vector, the patient exhibits a level of PGRN expression in the frontal cortex that is equivalent to a level of PGRN expression observed in a human subject having a neurocognitive or a neuromuscular disorder (e.g., a neurodegenerative disorder such as FTD, AD, PD, dementia with Lewy bodies, a related neurocognitive disorder, ALS, or a related motor neuron disorder) following intrathalamic administration, in an amount of from about 1×10⁹ vg/hemisphere to about 9×10¹² vg/hemisphere (e.g., about 5×10⁹ vg/hemisphere to about 1×10¹² vg/hemisphere, about 1×10¹⁰ vg/hemisphere to about 5×10¹¹ vg/hemisphere, or about 5×10¹⁰ vg/hemisphere to about 1×10¹¹ vg/hemisphere), of an AAV2/9 vector having the nucleic acid sequence of SEQ ID NO: 6. For example, in some embodiments, following administration of the AAV vector, the patient exhibits a level of PGRN expression in the frontal cortex that is equivalent to a level of PGRN expression observed in a human subject having a neurocognitive or a neuromuscular disorder (e.g., a neurodegenerative disorder such as FTD, AD, PD, dementia with Lewy bodies, a related neurocognitive disorder, ALS, or a related motor neuron disorder) following intrathalamic administration, in an amount of from about 5×10⁹ vg/hemisphere to about 1×10¹² vg/hemisphere of an AAV2/9 vector having the nucleic acid sequence of SEQ ID NO: 6. In some embodiments, following administration of the AAV vector, the patient exhibits a level of PGRN expression in the frontal cortex that is equivalent to a level of PGRN expression observed in a human subject having a neurocognitive or a neuromuscular disorder (e.g., a neurodegenerative disorder such as FTD, AD, PD, dementia with Lewy bodies, a related neurocognitive disorder, ALS, or a related motor neuron disorder) following intrathalamic administration, in an amount of from about 1×10¹⁰ vg/hemisphere to about 5×10¹¹ vg/hemisphere of an AAV2/9 vector having the nucleic acid sequence of SEQ ID NO: 6. In some embodiments, following administration of the AAV vector, the patient exhibits a level of PGRN expression in the frontal cortex that is equivalent to a level of PGRN expression observed in a human subject having a neurocognitive or a neuromuscular disorder (e.g., a neurodegenerative disorder such as FTD, AD, PD, dementia with Lewy bodies, a related neurocognitive disorder, ALS, or a related motor neuron disorder) following intrathalamic administration, in an amount of from about 5×10¹⁰ vg/hemisphere to about 1×10¹¹ vg/hemisphere of an AAV2/9 vector having the nucleic acid sequence of SEQ ID NO: 6.

In some embodiments, following ITM administration of the AAV vector, the patient exhibits no significant increase in expression of PGRN in one or more (e.g., two, three, four, or more) peripheral tissues (e.g., the liver, the lung, and the spleen), such as an increase of less than about 10% (e.g., less than about 9%, less than about 8%, less than about 7%, less than about 6%, less than about 5%, less than about 4%, less than about 3%, less than about 2%, or less than about 1%, such as 0%). For example, in some embodiments, following administration of the AAV vector, the patient exhibits an increase in the expression of PGRN in one or more (e.g., two, three, four, or more) peripheral tissues (e.g., the liver, the lung, and the spleen) of less than about 9%. In some embodiments, following administration of the AAV vector, the patient exhibits an increase in the expression of PGRN in one or more (e.g., two, three, four, or more) peripheral tissues (e.g., the liver, the lung, and the spleen) of less than about 8%. In some embodiments, following administration of the AAV vector, the patient exhibits an increase in the expression of PGRN in one or more (e.g., two, three, four, or more) peripheral tissues (e.g., the liver, the lung, and the spleen) of less than about 7%. In some embodiments, following administration of the AAV vector, the patient exhibits an increase in the expression of PGRN in one or more (e.g., two, three, four, or more) peripheral tissues (e.g., the liver, the lung, and the spleen) of less than about 6%. In some embodiments, following administration of the AAV vector, the patient exhibits an increase in the expression of PGRN in one or more (e.g., two, three, four, or more) peripheral tissues (e.g., the liver, the lung, and the spleen) of less than about 5%. In some embodiments, following administration of the AAV vector, the patient exhibits an increase in the expression of PGRN in one or more (e.g., two, three, four, or more) peripheral tissues (e.g., the liver, the lung, and the spleen) of less than about 4%. In some embodiments, following administration of the AAV vector, the patient exhibits an increase in the expression of PGRN in one or more (e.g., two, three, four, or more) peripheral tissues (e.g., the liver, the lung, and the spleen) of less than about 3%. In some embodiments, following administration of the AAV vector, the patient exhibits an increase in the expression of PGRN in one or more (e.g., two, three, four, or more) peripheral tissues (e.g., the liver, the lung, and the spleen) of less than about 2%. In some embodiments, following administration of the AAV vector, the patient exhibits an increase in the expression of PGRN in one or more (e.g., two, three, four, or more) peripheral tissues (e.g., the liver, the lung, and the spleen) of less than about 1%. In some embodiments, following administration of the AAV vector, the patient exhibits an increase in the expression of PGRN in one or more (e.g., two, three, four, or more) peripheral tissues (e.g., the liver, the lung, and the spleen) of 0%

In some embodiments, the peripheral tissues include, but are not limited to, the liver, the lung, and the spleen.

In some embodiments, the expression of PGRN is measured relative to the expression of GAPDH.

### Kits

The compositions described herein can be provided in a kit for use in treating a neurocognitive or a neuromuscular disorder (e.g., a neurodegenerative disorder such as FTD, AD, PD, dementia with Lewy bodies, a related neurocognitive disorder, ALS, or a related motor neuron disorder). In some embodiments, the kit may include one or more AAV as described herein. The kit can include a package insert that instructs a user of the kit, such as a physician of skill in the art, to perform any one of the methods described herein. The kit may optionally include a syringe or other device for administering the composition. In some embodiments, the kit may include one or more additional therapeutic agents.

### EXAMPLES

The following examples are put forth so as to provide those of ordinary skill in the art with a description of how the compositions and methods described herein may be used and evaluated and are intended to be purely exemplary of the invention and are not intended to limit the scope of what the inventors regard as their invention.

### Example 1. Development of Codon-Optimized Human Progranulin Constructs for Efficacious Cortical Expression of Progranulin

Codon optimization of the GRN gene for efficacious protein expression of human progranulin (hPGRN)

The human GRN gene sequence, excluding intronic DNA, is as follows:

(SEQ ID NO: 10)
ATTCTCCAATCACATGATCCCTAGAAATGGGGTGTGGGGCGAGAGGAAG
CAGGGAGGAGAGTGATTTGAGTAGAAAAGAAACACAGCATTCCAGGCTG
GCCCCACCTCTATATTGATAAGTAGCCAATGGGAGCGGGTAGCCCTGAT
CCCTGGCCAATGGAAACTGAGGTAGGCGGGTCATCGCGCTGGGGTCTGT
AGTCTGAGCGCTACCCGGTTGCTGCTGCCCAAGGACCGCGGAGTCGGAC
GCAGGCAGACCATGTGGACCCTGGTGAGCTGGGTGGCCTTAACAGCAGG
GCTGGTGGCTGGAACGCGGTGCCCAGATGGTCAGTTCTGCCCTGTGGCC
TGCTGCCTGGACCCCGGAGGAGCCAGCTACAGCTGCTGCCGTCCCCTTC
TGGACAAATGGCCCACAACACTGAGCAGGCATCTGGGTGGCCCCTGCCA
GGTTGATGCCCACTGCTCTGCCGGCCACTCCTGCATCTTTACCGTCTCA
GGGACTTCCAGTTGCTGCCCCTTCCCAGAGGCCGTGGCATGCGGGGATG
GCCATCACTGCTGCCCACGGGGCTTCCACTGCAGTGCAGACGGGCGATC
CTGCTTCCAAAGATCAGGTAACAACTCCGTGGGTGCCATCCAGTGCCCT
GATAGTCAGTTCGAATGCCCGGACTTCTCCACGTGCTGTGTTATGGTCG
ATGGCTCCTGGGGGTGCTGCCCCATGCCCCAGGCTTCCTGCTGTGAAGA
CAGGGTGCACTGCTGTCCGCACGGTGCCTTCTGCGACCTGGTTCACACC
CGCTGCATCACACCCACGGGCACCCACCCCCTGGCAAAGAAGCTCCCTG
CCCAGAGGACTAACAGGGCAGTGGCCTTGTCCAGCTCGGTCATGTGTCC
GGACGCACGGTCCCGGTGCCCTGATGGTTCTACCTGCTGTGAGCTGCCC
AGTGGGAAGTATGGCTGCTGCCCAATGCCCAACGCCACCTGCTGCTCCG
ATCACCTGCACTGCTGCCCCCAAGACACTGTGTGTGACCTGATCCAGAG
TAAGTGCCTCTCCAAGGAGAACGCTACCACGGACCTCCTCACTAAGCTG
CCTGCGCACACAGTGGGGGATGTGAAATGTGACATGGAGGTGAGCTGCC
CAGATGGCTATACCTGCTGCCGTCTACAGTCGGGGGCCTGGGGCTGCTG
CCCTTTTACCCAGGCTGTGTGCTGTGAGGACCACATACACTGCTGTCCC
GCGGGGTTTACGTGTGACACGCAGAAGGGTACCTGTGAACAGGGGCCCC
ACCAGGTGCCCTGGATGGAGAAGGCCCCAGCTCACCTCAGCCTGCCAGA
CCCACAAGCCTTGAAGAGAGATGTCCCCTGTGATAATGTCAGCAGCTGT
CCCTCCTCCGATACCTGCTGCCAACTCACGTCTGGGGAGTGGGGCTGCT
GTCCAATCCCAGAGGCTGTCTGCTGCTCGGACCACCAGCACTGCTGCCC
CCAGGGCTACACGTGTGTAGCTGAGGGGCAGTGTCAGCGAGGAAGCGAG
ATCGTGGCTGGACTGGAGAAGATGCCTGCCCGCCGGGCTTCCTTATCCC
ACCCCAGAGACATCGGCTGTGACCAGCACACCAGCTGCCCGGTGGGGCA
GACCTGCTGCCCGAGCCTGGGTGGGAGCTGGGCCTGCTGCCAGTTGCCC
CATGCTGTGTGCTGCGAGGATCGCCAGCACTGCTGCCCGGCTGGCTACA
CCTGCAACGTGAAGGCTCGATCCTGCGAGAAGGAAGTGGTCTCTGCCCA
GCCTGCCACCTTCCTGGCCCGTAGCCCTCACGTGGGTGTGAAGGACGTG
GAGTGTGGGGAAGGACACTTCTGCCATGATAACCAGACCTGCTGCCGAG
ACAACCGACAGGGCTGGGCCTGCTGTCCCTACCGCCAGGGCGTCTGTTG
TGCTGATCGGCGCCACTGCTGTCCTGCTGGCTTCCGCTGCGCAGCCAGG
GGTACCAAGTGTTTGCGCAGGGAGGCCCCGCGCTGGGACGCCCCTTTGA
GGGACCCAGCCTTGAGACAGCTGCTGTGAGGGACAGTACTGAAGACTCT
GCAGCCCTCGGGACCCCACTCGGAGGGTGCCCTCTGCTCAGGCCTCCCT
AGCACCTCCCCCTAACCAAATTCTCCCTGGACCCCATTCTGAGCTCCCC
ATCACCATGGGAGGTGGGGCCTCAATCTAAGGCCTTCCCTGTCAGAAGG
GGGTTGTGGCAAAAGCCACATTACAAGCTGCCATCCCCTCCCCGTTTCA
GTGGACCCTGTGGCCAGGTGCTTTTCCCTATCCACAGGGGTGTTTGTGT
GTGTGCGCGTGTGCGTTTCAATAAAGTTTGTACACTTTCTTAAAAAAAA
AAAA.

The human PGRN amino acid sequence is as follows:

(SEQ ID NO: 2)
MWTLVSWVALTAGLVAGTRCPDGQFCPVACCLDPGGASYSCCRPLLDKW
PTTLSRHLGGPCQVDAHCSAGHSCIFTVSGTSSCCPFPEAVACGDGHHC
CPRGFHCSADGRSCFQRSGNNSVGAIQCPDSQFECPDFSTCCVMVDGSW
GCCPMPQASCCEDRVHCCPHGAFCDLVHTRCITPTGTHPLAKKLPAQRT
NRAVALSSSVMCPDARSRCPDGSTCCELPSGKYGCCPMPNATCCSDHLH
CCPQDTVCDLIQSKCLSKENATTDLLTKLPAHTVGDVKCDMEVSCPDGY
TCCRLQSGAWGCCPFTQAVCCEDHIHCCPAGFTCDTQKGTCEQGPHQVP
WMEKAPAHLSLPDPQALKRDVPCDNVSSCPSSDTCCQLTSGEWGCCPIP
EAVCCSDHQHCCPQGYTCVAEGQCQRGSEIVAGLEKMPARRASLSHPRD
IGCDQHTSCPVGQTCCPSLGGSWACCQLPHAVCCEDRQHCCPAGYTCNV
KARSCEKEVVSAQPATFLARSPHVGVKDVECGEGHFCHDNQTCCRDNRQ
GWACCPYRQGVCCADRRHCCPAGFRCAARGTKCLRREAPRWDAPLRDPA
LRQLL.

Analysis of SEQ ID NO: 10 reveals specific codon preferences for various amino acids throughout the gene. Inspection of the codon frequencies reveals that for certain amino acids, a particular codon is predominantly while other codons are used less frequently or not at all. One of skill in the art can perform codon optimization in any manner known in the art, for example, such as a manner such as that described in, e.g., U.S. Pat. Nos. 7,561,972, 7,561,973, and 7,888,112, each of which is incorporated herein by reference in its entirety.

The final codon-optimized gene may exhibit at least 85% sequence identity to the nucleic acid sequence of SEQ ID NO: 3. For example, the final codon-optimized gene may exhibit at least 86%, 87%, 88%, 89%, 90%, 91%, 92%, 93%, 94%, 95%, 96%, 97%, 98%, or 99% sequence identity to the nucleic acid sequence of SEQ ID NO: 3. In another example, the final codon-optimized gene may have a nucleic acid sequence that is identical to the nucleic sequence of SEQ ID NO: 3.

Once designed, the final codon-optimized gene can be prepared, for instance, by solid phase nucleic acid procedures known in the art. Techniques for the solid phase synthesis of polynucleotides are known in the art and are described, for instance, in U.S. Pat. No. 5,541,307, the disclosure of which is incorporated herein by reference as it pertains to solid phase polynucleotide synthesis and purification. Additionally, the prepared gene can be amplified, for instance, using PCR-based techniques known in the art, and/or by transformation of DH5a E. coli with a plasmid containing the designed gene. The bacteria can subsequently be cultured so as to amplify the DNA therein, and the gene can be isolated by plasmid purification techniques known in the art, followed optionally by a restriction digest and/or sequencing of the plasmid to verify the identity codon-optimized gene.

### Codon-Optimized Human PGRN Constructs

PCR primers were designed to bind to and amplify the GRN nucleic acid sequence from genomic DNA isolated from cultured cells by polymerase chain reaction. Using the codon-optimization methods described above, the isolated GRN was modified by site-directed mutagenesis. The resulting modified amplification product was gel purified and sequenced by methods known in the art.

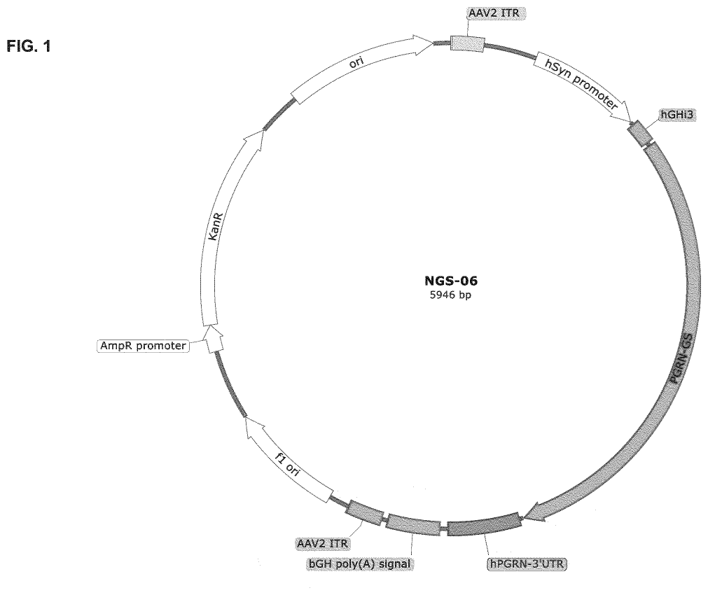

A pseudotyped adeno-associated virus (AAV) 2/9 (AAV 2/9) parental vector was used as the destination vector for the codon optimized human PGRN (hPGRN). The parental vector contains a nucleic acid molecule having the following components: a first AAV2 inverted terminal repeat, a human synapsin (hSyn) promoter, a human growth hormone intron (hGHi3), a bovine growth hormone (bGH) polyadenylation site (pA), a second AAV2 ITR (ITR2), a phage-derived origin of replication (f1 ori), a Citrobacter freundii ampC B-lactamase (AmpR) promoter, a kanamycin selection gene (KanR), and a second origin of replication (ori). The codon-optimized hPGRN was cloned into the parental vector (FIG. 1; referenced to herein as “AAV9-SYN-PGRN”).

### Example 2. Establishing Therapeutic Expression of hPGRN in the Cortex while Avoiding Toxic Side Effects

### Objective

The objective of this study was to evaluate the anterograde and/or retrograde trafficking together with systemic toxicity and/or expression of AAV9-SYN-PGRN, as described in Example 1, in adult sheep for the rescued expression of PGRN in the cortex. Furthermore, this study was designed to refine the effective dosing range of AAV9-SYN-PGRN, as identified by supraphysiological levels of PGRN in cortical brain tissues.

### Materials and Methods

Adult (approximately 2 years old) wild-type sheep (n=2/dose) were intrathalamically (ITM) infused (e.g., convection-assisted administration) with 1×10¹⁰ vg/hemisphere (referred to herein as “low dose”), 5×10¹⁰ vg/hemisphere (referred to herein as “mid dose”), or 1×10¹¹ vg/hemisphere (referred to herein as “high dose”) of AAV9-SYN-PGRN (250 pL/thalamus) over approximately 90 minutes, respectively. 28 days post ITM administration, animals were sacrificed, and brain biopsies were collected. The brain, cerebrospinal fluid (CSF), and serum were analyzed for hPGRN protein expression and vg levels, whilst immunofluorescence imaging of the brain was performed to evaluate hPGRN expression and potential inflammatory response (e.g., expression of inflammatory markers) to the viral vector.

### Results

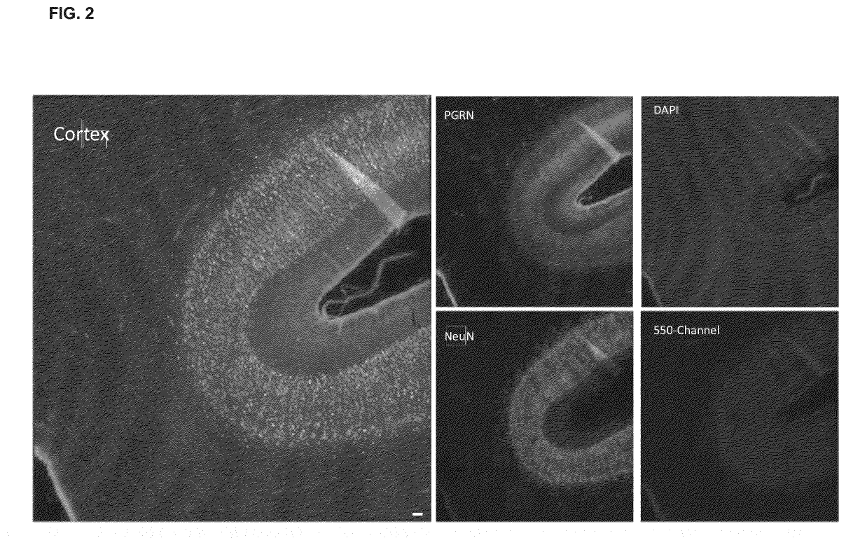

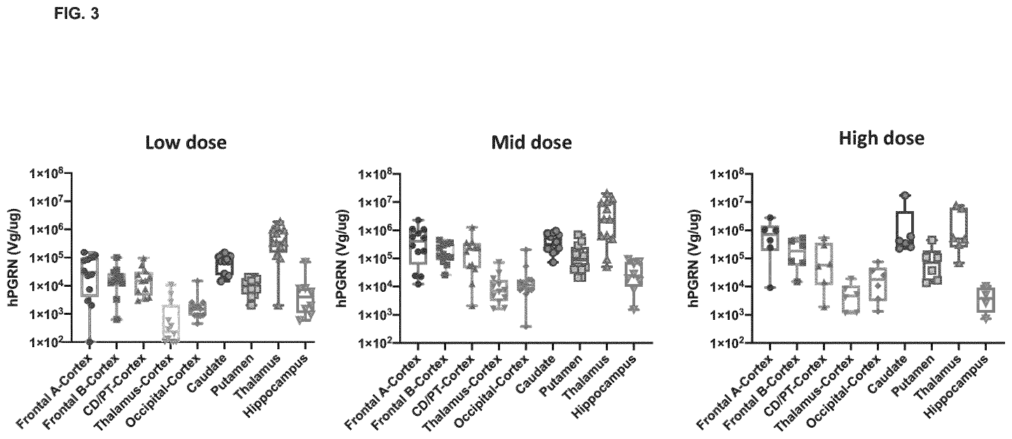

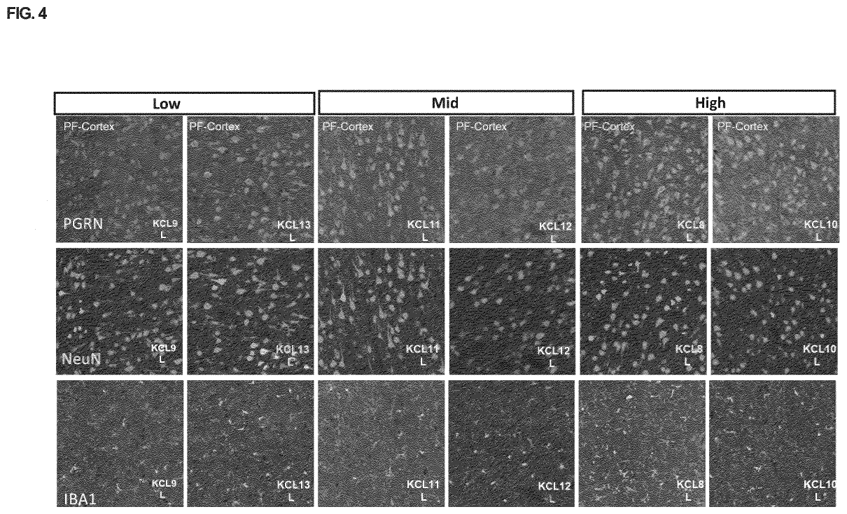

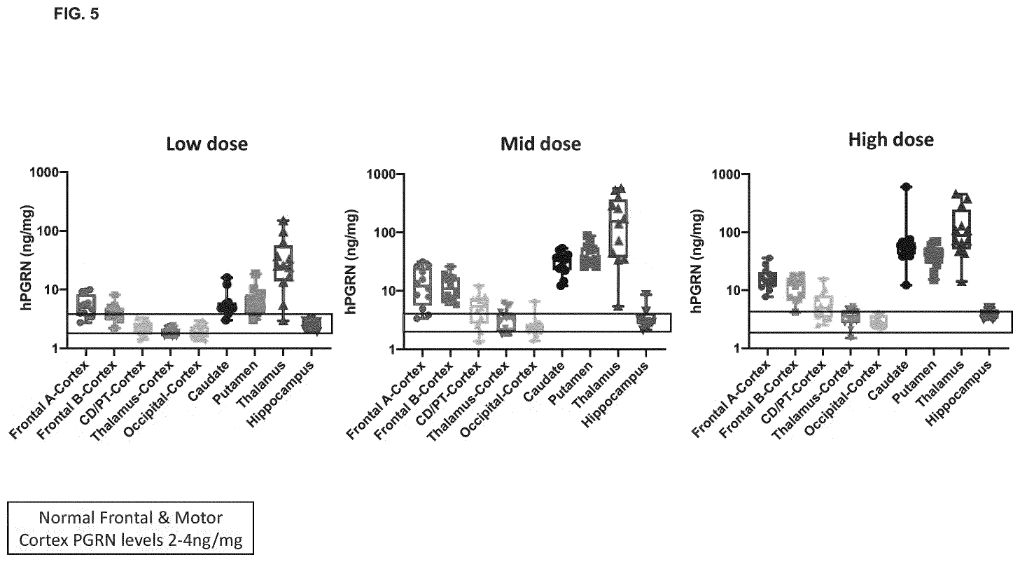

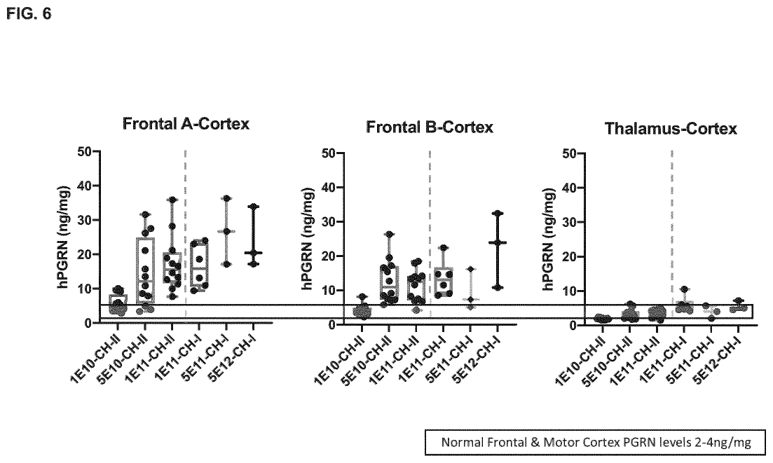

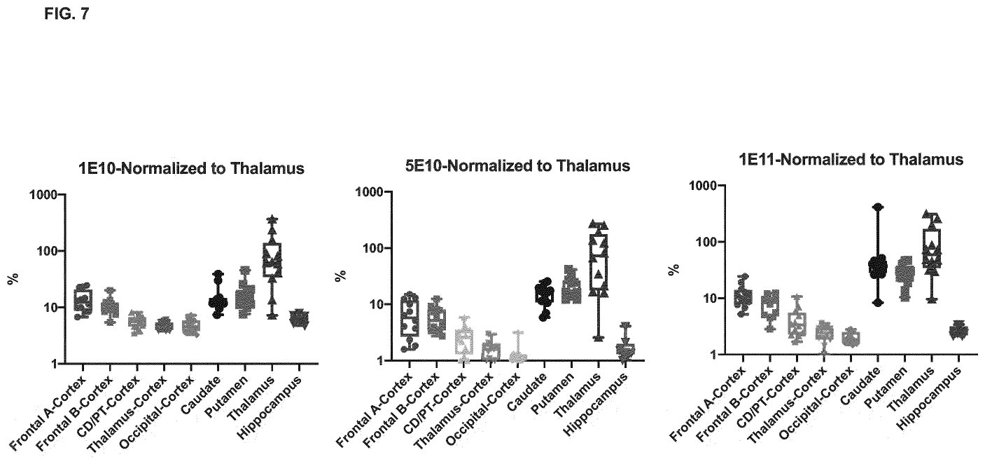

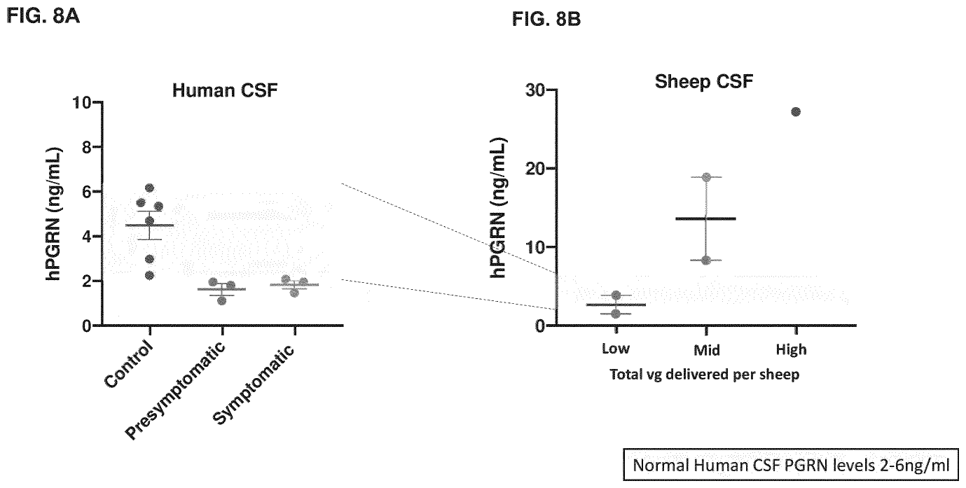

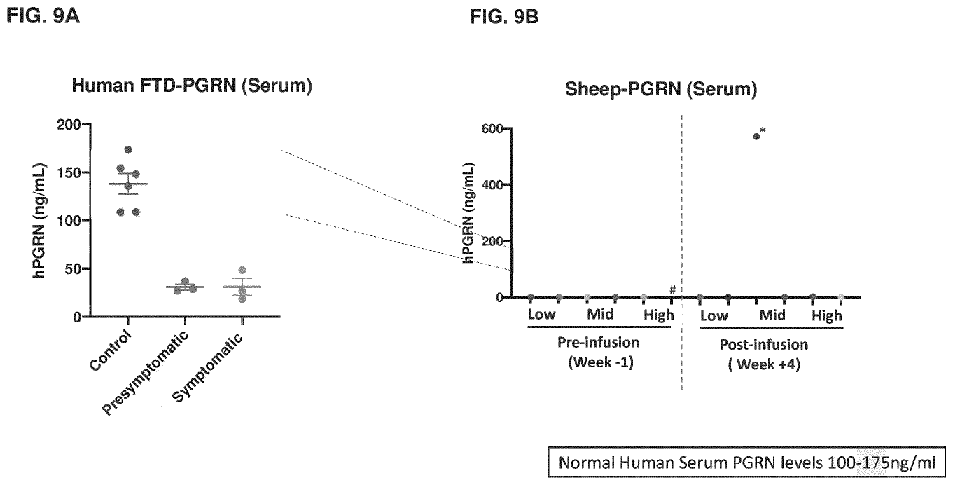

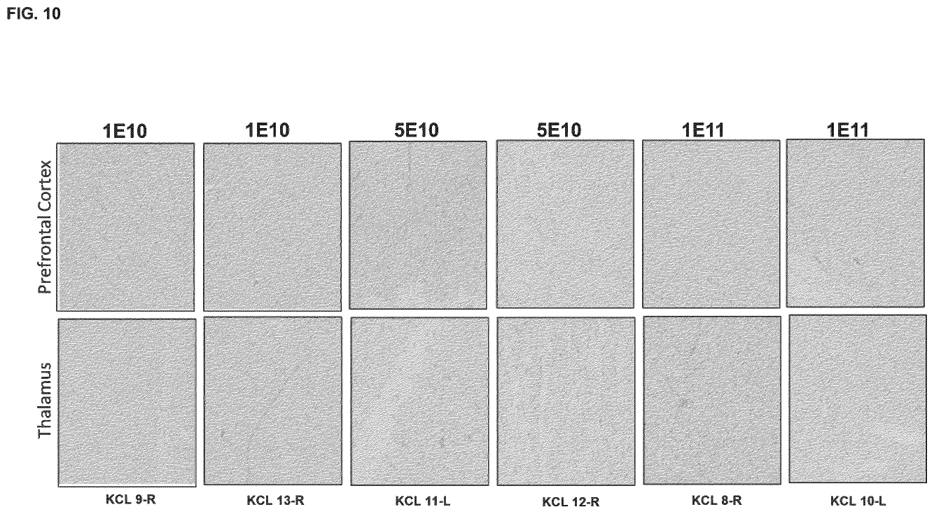

In result, we observed that the vector preparation described herein, as infused into the thalamus, effectively transduced the cortex (FIG. 2), with normal to supraphysiological levels of hPGRN in the cortex even at the low dose. Further, we observed a dose-dependent increase in hPGRN levels in all brain areas and a high animal-to-animal consistency (FIG. 3). This effect was observed across the prefrontal (PF) cortex of all sheep, and no evidence of reactive microglia were observed, as measured by the expression levels of IBA1 (FIG. 4). In evaluating the dose-expression response across cortical regions (e.g., Frontal A cortex (coronal slices taken from the anterior portion of the cortex), Frontal B cortex (coronal slices taken from the posterior portion of the cortex), caudate putamen/parietal temporal (CD/PT) cortex), the basal ganglia (e.g., the caudate and putamen), the thalamus, and the hippocampus, we observed that even the lowest dose tested elevated hPGRN protein expression above basal levels (FIGS. 5 and 6). Furthermore, upon normalizing this data to the percentage of hPGRN expression in the thalamus, we observed that the low dose trended towards greater relative hPGRN delivery to the cortex (FIG. 7). In the CSF, the mid and high doses elevated hPGRN levels to achieve supraphysiological human CSF PGRN levels in the 10-30 ng/ml range, indicating a restorative effect (FIG. 8). No detectable expression of hPGRN was observed in the serum of said respective sheep (FIG. 9). In a study to address histopathology, hematoxylin and eosin staining revealed that all animals showed a unilateral or bilateral focal inflammatory lesion in the cerebral white matter and/or thalamus, consistent with damage caused during dose administration. There was no evidence of histopathological changes specifically at the sites of hPGRN expression (FIG. 10). Gliosis was observed in a few animals, though considered unrelated to expression of hPGRN in view of its minimal, focal nature, and sporadic occurrence in both white matter and grey matter in different regions. This was likely also associated with dosing and/or a spontaneous background change. Taken together, these data demonstrated that at all dose levels, AAV9-SYN-PGRN effectively transduced the cortex and mediated elevated cortical hPGRN expression levels, without eliciting an immunological response against the AAV vector.

### Example 3. Establishing Dosing Efficiency of hPGRN in the Cortex

### Objective

The objective of this study was to examine the efficiency of cortical transduction by ITM administration of AAV9-SYN-PGRN in adult sheep.

### Materials and Methods

Materials and Methods are described in Examples 1 and 2.

### Results

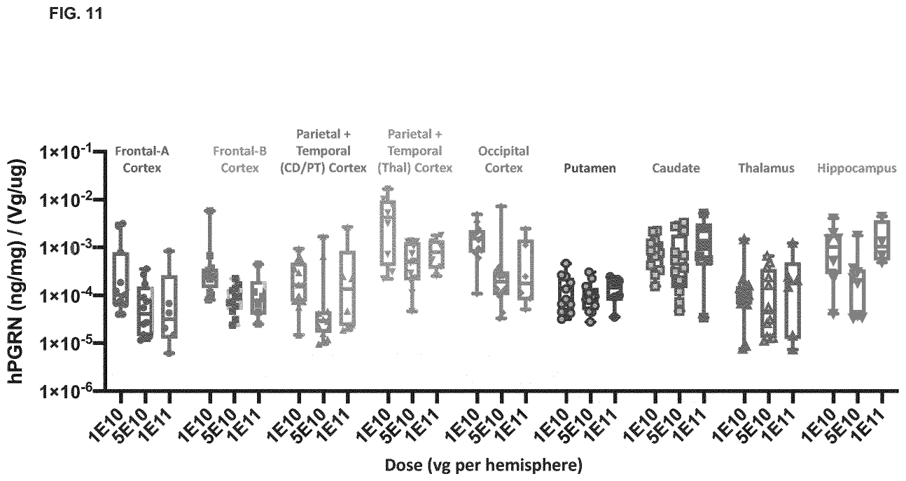

FIG. 11 shows the expression level of hPGRN, as normalized by the quantified vg/μg of AAV9-SYN-PGRN (FIG. 3), across brain regions in wild-type sheep administered ITM with the low, mid, or high dose, respectively. We observed that the low dose was generally more efficient at delivering hPGRN to the cortex.

### Example 4. Comparing Efficiency of hPGRN Transduction in the Cortex Across Central Administration Routes

### Objective

The objective of this study was to compare the efficiency of cortical transduction by ITM or intra cistern magma (ICM) administration of viral vectors encoding a hPGRN transgene in adult sheep.

### Materials and Methods

Adult (approximately 2 years old) wild-type sheep (n=2/dose or 2/vector, respectively) were infused ITM (e.g., convection-assisted administration) with 1×10¹⁰ (250 μL/thalamus over approximately 90 minutes) vg/hemisphere or ICM with 1×10¹³ vg (2 mL over approximately 1 minute with CSF replacement) of AAV9-SYN-PGRN or an AAV1 or AAV9 encoding a hPGRN transgene operably linked to a chicken β-actin promoter with a cytomegalovirus enhancer (CB7; AAV1-CB7-PGRN and AAV9-CB7-PGRN, respectively) or control Omnipaque, respectively. Regions from the brain were biopsied and analyzed for hPGRN protein expression and vg levels, whilst immunofluorescence imaging of the brain was performed to evaluate hPGRN expression.

### Results

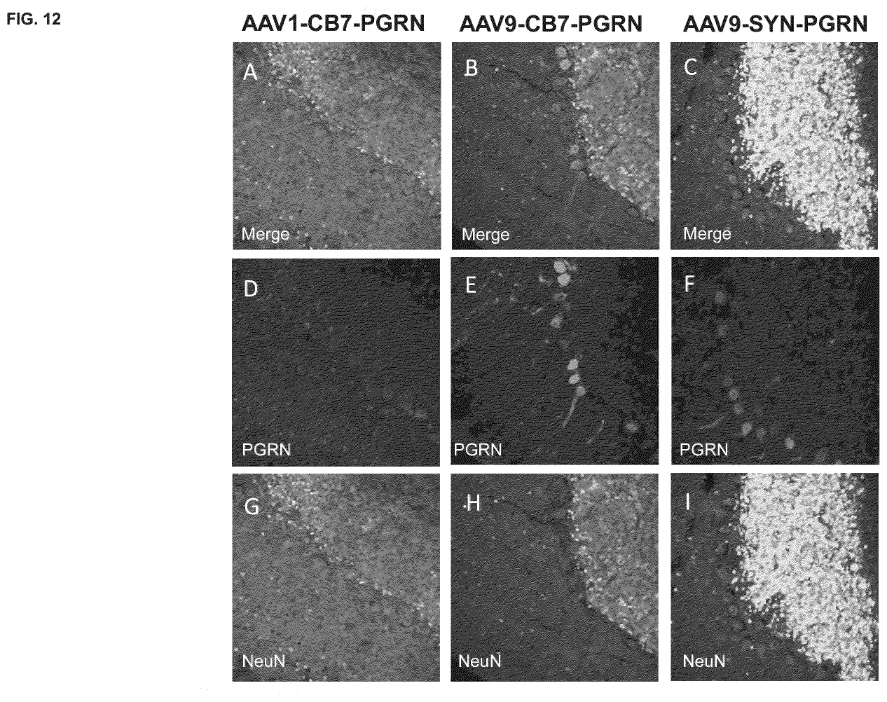

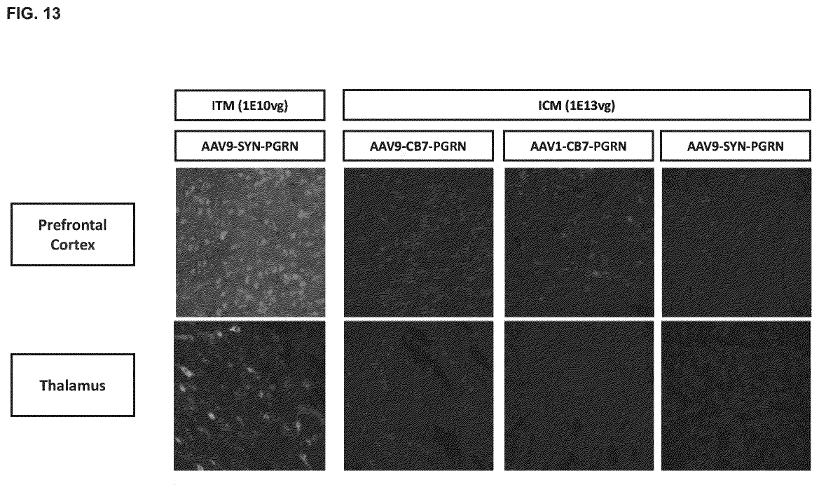

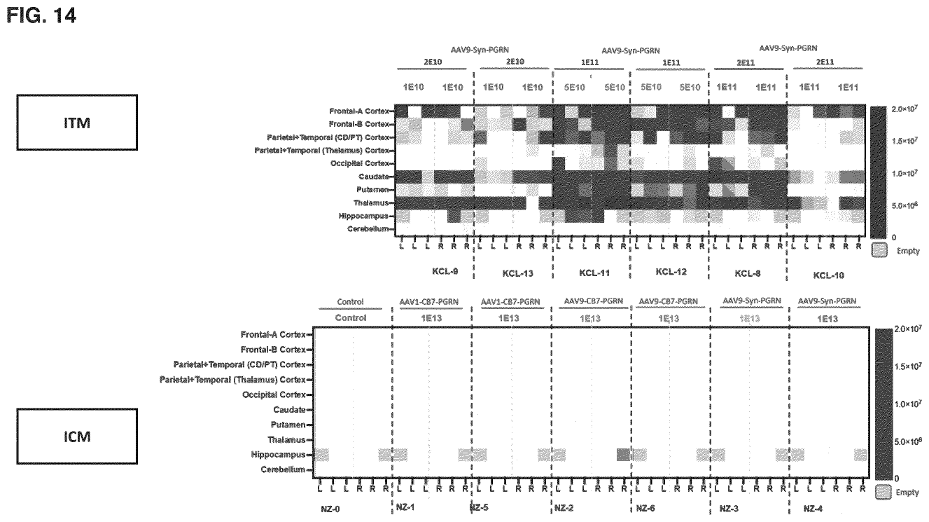

Initial evaluation of cerebellar sections for hPGRN confirms successful ICM administration (FIG. 12). All animals showed hPGRN in the cerebellum, though differences were observed. AAV1-CB7-PGRN demonstrated intense, superficial, and almost exclusively glial staining. Both AAV9-SYN-PGRN and AAV9-CB7-PGRN demonstrated better penetration of hPGRN into cerebellar tissues, with AAV9-CB7-PGRN being more prevalent in glial cells and AAV9-SYN-PGRN more prevalent in neurons. In the PF cortex and thalamus, ICM administration mediated little expression independent of which AAV vector was used (FIG. 13). In contrast, this comparative study highlighted that ITM administration mediated strong expression of hPGRN in the PF cortex. Similar results were obtained across brain regions, in that ITM administration yielded enhanced transduction, as compared to ICM administration (FIG. 14).

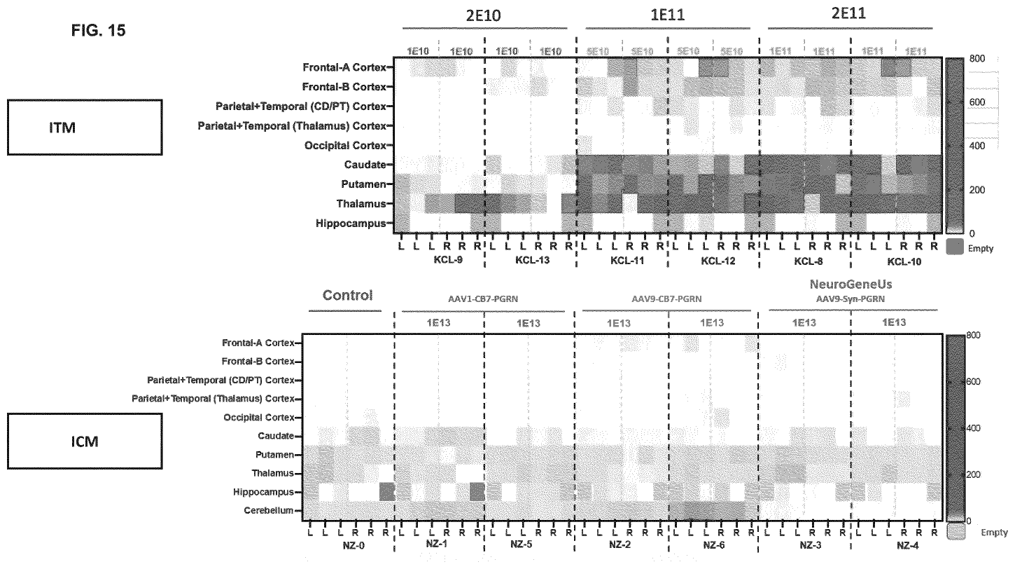

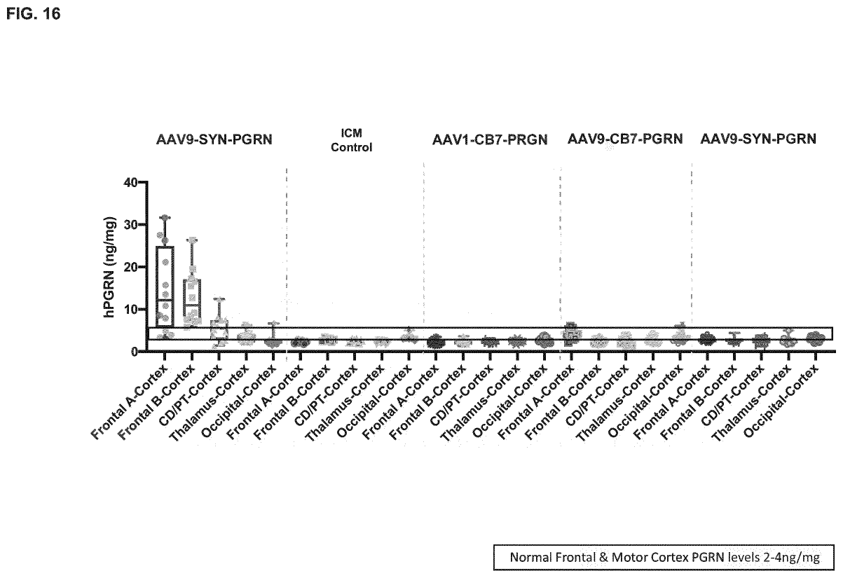

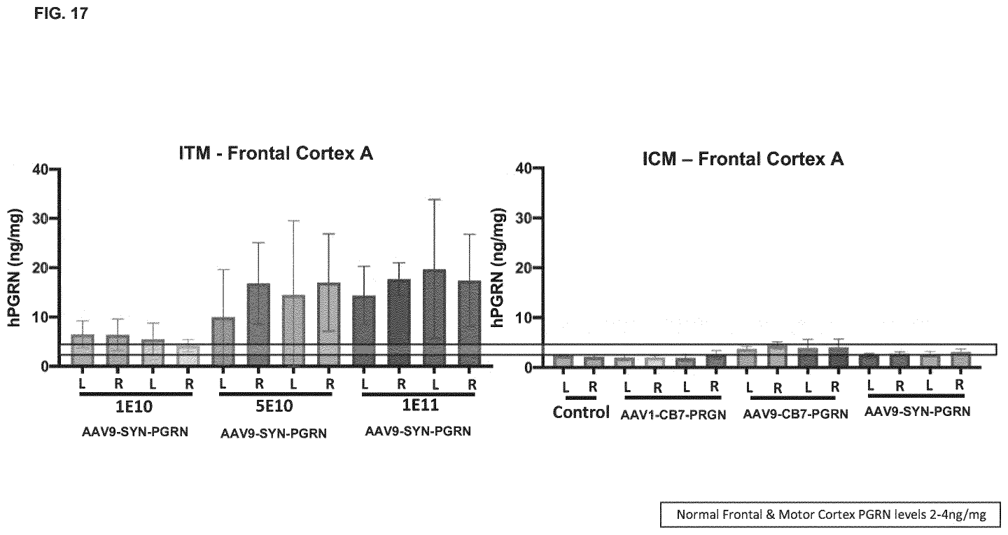

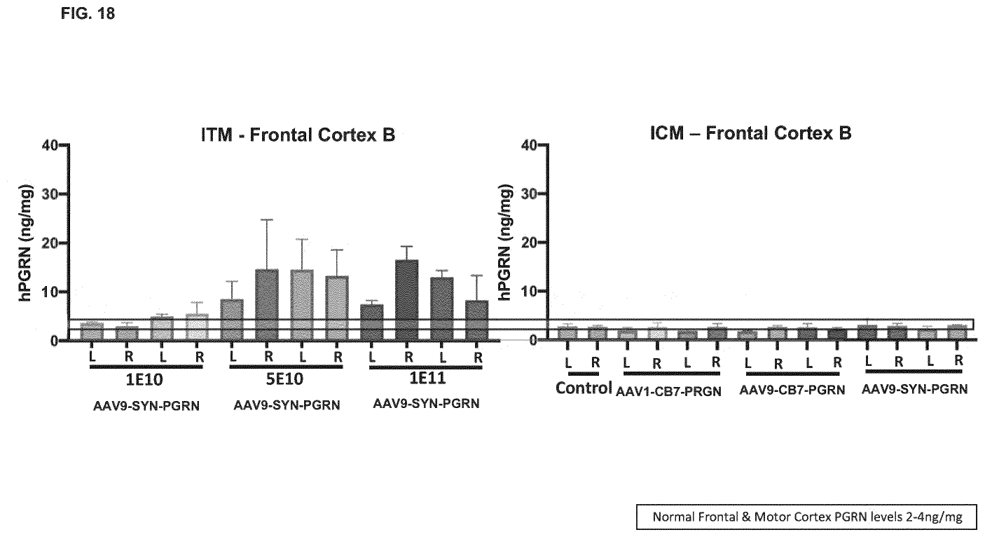

In evaluating the level of hPGRN protein expression, we observed a similar pattern of results. Specifically, we observed that the levels detected in the cortex and subcortex of ICM treated sheep were within the background levels detected in control (untreated sheep), while sheep infused ITM with 5×10¹⁰ vg/hemisphere of AAV9-SYN-PGRN demonstrated greater cortical levels of hPGRN compared with sheep administered ICM AAV9-SYN-PGRN or AAV1-CB7-PGRN or AAV9-CB7-PGRN vectors (FIGS. 15-18). In the cortex of ICM infused animals, we observed negligible hPGRN levels, ranging to small pockets of hPGRN detected in thalamus and parietal/temporal cortex of ICM treated sheep

Taken together, these results indicates that ICM administration yields poor transduction of the cortex while ITM administration achieves superior cortical transduction.

### Example 5. Evaluation of Tissue Expression of hPGRN in Cortical Tissue Compared to Peripheral Tissues after AAV Delivery Via ITM Administration of Viral Vectors Encoding a hPGRN Transgene in Adult Sheep

### Objective

The objective of this study was to evaluate hPGRN expression in the central nervous system (CNS) compared to peripheral tissues (e.g., the liver, the spleen, and the lung) after ITM administration of viral vectors encoding a hPGRN transgene in adult sheep.

### Materials and Methods

Adult (approximately 2 years old) wild-type sheep (n=2/dose or 2/vector, respectively, and one control sheep) were infused ITM (e.g., convection-assisted administration) with 1×10¹⁰, 5×10¹⁰, or 1×10¹¹ (250 μL/thalamus over approximately 90 minutes) vg/hemisphere of AAV9-SYN-PGRN or an AAV1 or AAV9 encoding a hPGRN transgene operably linked to a chicken β-actin promoter with a cytomegalovirus enhancer (CB7; AAV1-CB7-PGRN and AAV9-CB7-PGRN, respectively) or control Omnipaque, respectively. Regions from the brain, liver, spleen, and lung were biopsied and analyzed for hPGRN protein expression and vg levels, whilst immunofluorescence imaging of the brain, liver, spleen, and lung were performed to evaluate hPGRN expression.

### Results

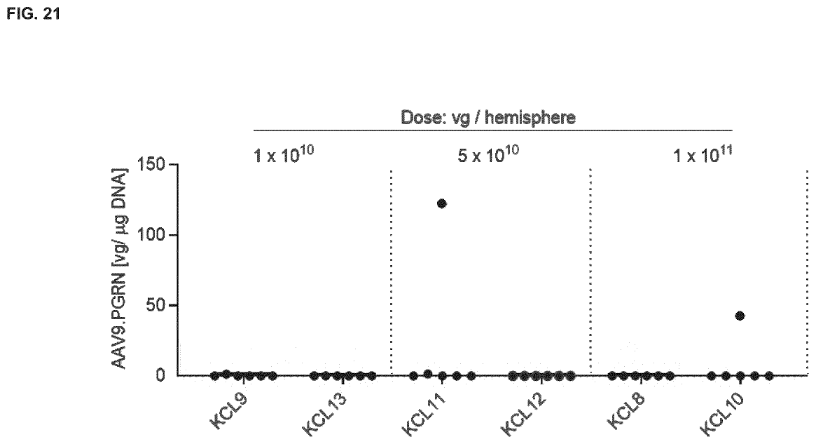

As discussed in Example 4, ITM administration of AAV9 vectors encoding a hPGRN transgene mediated strong expression of hPGRN in the brain, including the thalamus, frontal cortex, parietal/temporal cortex, occipital cortex, putamen, caudate, hippocampus, and serum from the left and right hemisphere. In evaluating the level of hPGRN protein expression, no significant expression of hPGRN (as assessed by viral DNA) was observed in peripheral tissues, including the liver (FIG. 21), lung, and spleen.

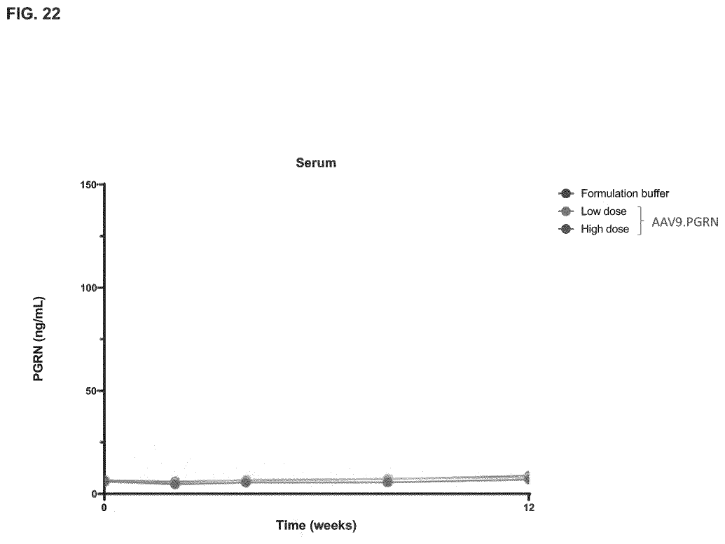

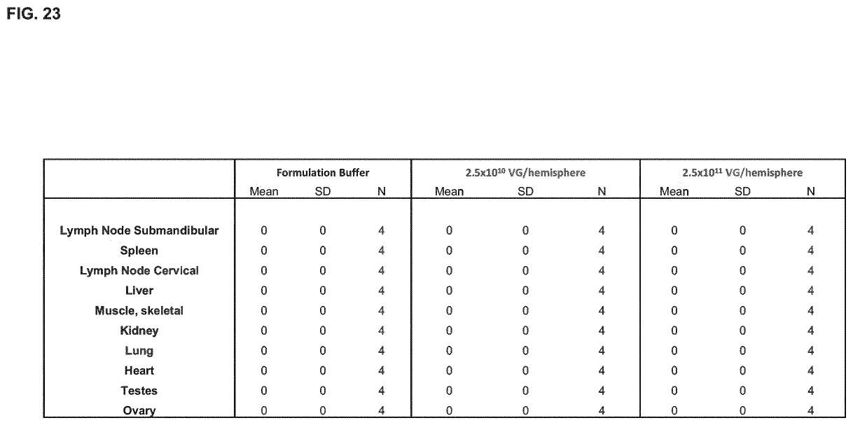

Notably, similar results have been obtained in cynomolgus monkeys (see, e.g., FIGS. 22 and 23).

Taken together, these results indicate that ITM administration of this vector achieves CNS-specific expression of hPGRN.

### Example 6. Effectiveness of Codon-Optimized Human PGRN on Lipofuscinosis in a Model of Frontotemporal Dementia

### Objective

Frontotemporal dementia (FTD) is a clinical syndrome characterized by progressive neurodegeneration in the frontal and temporal lobes of the cerebral cortex. Over 70 loss-of-function mutations in the GRN gene have been identified in FTD, the vast majority of which result in haploinsufficiency and a reduction in serum PGRN levels.

The objective of this study was to demonstrate the efficacy of AAV9-SYN-PGRN in a mouse model of early disease (e.g., FTD).

### Materials and Methods

### Frontotemporal Dementia Mouse Model

Mice homozygous for the deletion of GRN (e.g., GRN^{-/-}mice) were used as a model for FTD. At the age of 6-8 weeks, mice were infused with AAV9-SYN-PGRN, as described in Example 1, and 12 weeks were allowed for viral expression.

### Immunofluorescence

Biopsied brain tissue was fixed in 10% formalin overnight, then preserved in 70% ethanol. The tissue was embedded in paraffin and cut in 5-μm sections.

For immunofluorescence analysis, tissues were permeabilized and slides were washed and blocked for 30 min with 5% rabbit serum. Sections were incubated with primary antibodies for anti-hPGRN, anti-Iba1, anti-CD68, and anti-Subunit C Mitochondrial ATP Synthase (SCMAS), respectively, for 1 hour at room temperature. Slides were incubated with secondary antibody for 30 min. Assessments included detailed quantification of SCMAS immunoreactivity.

### Results

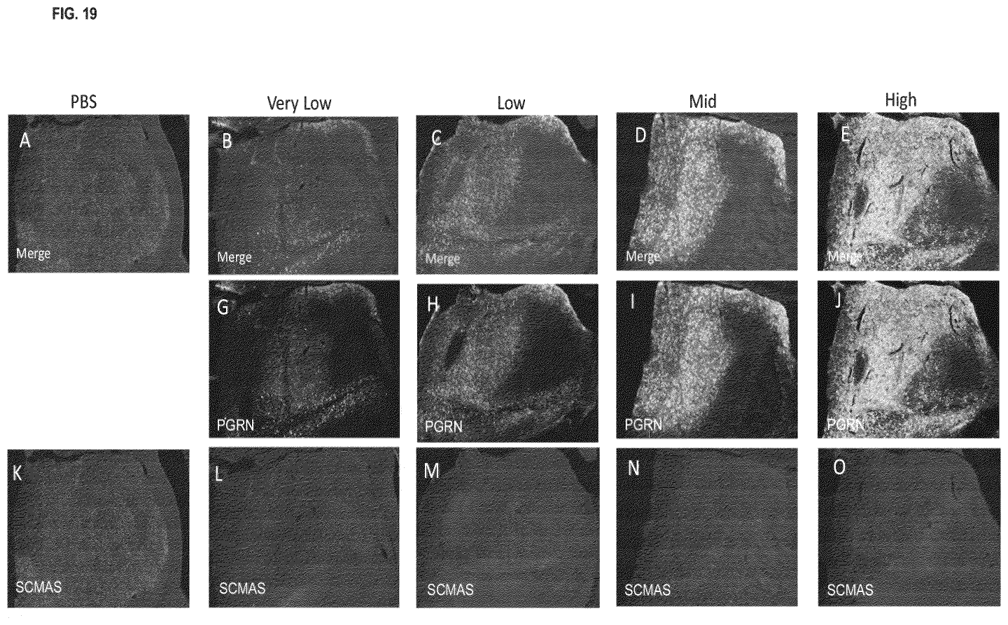

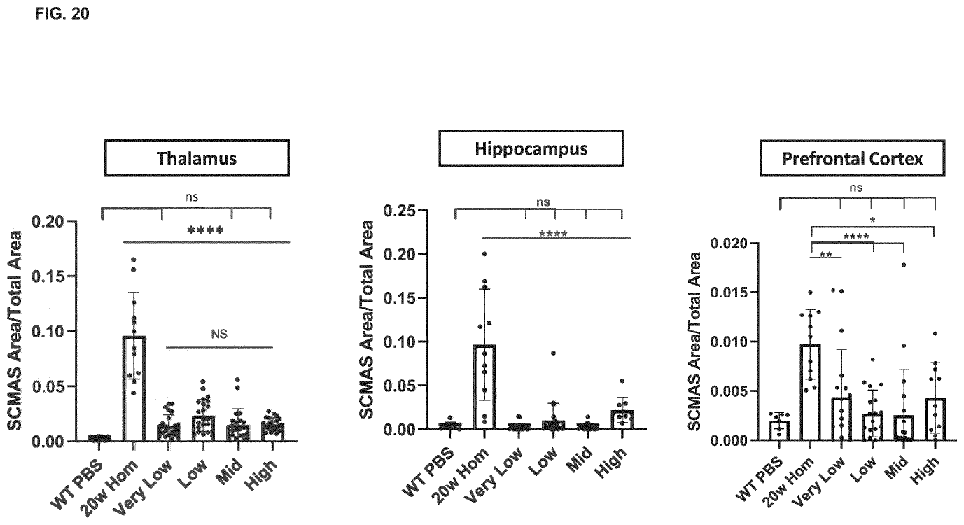

In result, we observed that reactive microglia (e.g., CD68- and IBA1-positive, amoeboid shaped cells) labelling were correlated with hPGRN levels. FIG. 19 shows that the SCMAS labeling of lipofuscin is reduced in regions even with low levels of hPGRN expression. This trend was apparent across all doses of AAV9-SYN-PGRN and correlated with regional expression (e.g., hPGRN-positive expression in the thalamus). Upon quantification, it was revealed that low doses of AAV9-SYN-PGRN significantly improve lipofuscinosis, as detected by SCMAS immunoreactivity (FIG. 20). Taken together, these results demonstrate the efficacy of an exemplary AAV2/9 plasmid encoding a codon-optimized hPGRN improved lipofuscinosis in the PF cortex, hippocampus, and thalamus in a murine model of FTD.

### Example 7. Use of a Codon-Optimized GRN Gene for the Treatment of a Neurocognitive or a Neuromuscular Disorder

A gene encoding PGRN can be codon-optimized using the procedures described herein (e.g., as described in Example 1, above). For example, the final codon-optimized GRN gene may exhibit at least 85% sequence identity to the nucleic acid sequence of SEQ ID NO: 3. For example, the final codon-optimized GRN gene may exhibit at least 85%, 86%, 87%, 88%, 89%, 90%, 91%, 92%, 93%, 94%, 95%, 96%, 97%, 98%, or 99% sequence identity to the nucleic acid sequence of SEQ ID NO: 3. In another example, the final codon-optimized gene may have a nucleic acid sequence that is identical to the nucleic sequence of SEQ ID NO: 3.

The gene can subsequently be incorporated into a plasmid, such as an AAV2/9 vector, and administered to a patient suffering from a neurocognitive or a neuromuscular disorder (e.g., a neurodegenerative disorder such as FTD, Alzheimer's disease (AD), Parkinson's disease (PD), dementia with Lewy bodies, a related neurocognitive disorder, amyotrophic lateral sclerosis (ALS), or a related motor neuron disorder). For instance, a patient suffering from FTD, a disorder associated by mutations in the GRN gene, or AD, PD, dementia with Lewy bodies, a related neurocognitive disorder, ALS, or a related motor neuron disorder can be administered an AAV2/9 vector containing a codon-optimized GRN gene under the control of a suitable promoter for expression in a human cell, such as a neuron. For instance, an AAV vector, such as a AAV2/9 vector, can be generated that incorporates the codon-optimized GRN gene between the 5′ and 3′ inverted terminal repeats of the vector, and the gene may be placed under control of a neuron-specific promoter, such as a synapsin (Syn) promoter. The AAV vector can be administered to the subject ITM.

A practitioner of skill in the art can monitor the expression of the codon-optimized GRN gene by a variety of methods. For instance, one of skill in the art can transfect cultured neurons with the codon-optimized gene in order to model the expression of the codon-optimized gene in the neurons of a patient. Expression of the encoded protein can subsequently be monitored using, for example, an expression assay described herein, such as qPCR, RNA-Seq, ELISA, or an immunoblot procedure. Based on the data obtained from the gene expression assay, further iterations of the codon optimization procedure can be performed, for instance, so as to further diminish CpG content and homopolymer content in the mRNA transcript. Candidate gene sequences with optimal expression patterns in vitro can subsequently be prepared for incorporation into a suitable AAV vector and administration to a mammalian subject, such as an animal model of a neurocognitive or a neuromuscular disorder (e.g., a neurodegenerative disorder such as FTD, AD, PD, dementia with Lewy bodies, a related neurocognitive disorder, ALS, or a related motor neuron disorder), or a human patient.

### Example 8. Treatment of a Neurocognitive or a Neuromuscular Disorder in Human Patients by Intrathalamic Administration of Human PGRN

Using the compositions and methods of the disclosure, a patient having a neurocognitive or a neuromuscular disorder (e.g., a neurodegenerative disorder such as FTD, AD, PD, dementia with Lewy bodies, a related neurocognitive disorder, ALS, or a related motor neuron disorder) may be administered (e.g., convection-assisted administration) ITM an AAV (e.g., a pseudotyped AAV2/9) vector including a nucleic acid sequence encoding a human PGRN. The human PGRN sequence may be codon-optimized and/or may operably be linked to a Syn promotor, for example, the AAV vector that has a nucleic acid of SEQ ID NO: 6. The AAV may be administered, for example, in an amount of from about 1×10⁹ vg/hemisphere to about 9×10¹² vg/hemisphere (e.g., 5×10⁹ vg/hemisphere to about 5×10¹² vg/hemisphere, 1×10¹⁰ vg/hemisphere to about 5×10¹² vg/hemisphere, 1×10¹¹ vg/hemisphere to about 5×10¹² vg/hemisphere, 1×10¹² vg/hemisphere to about 5×10¹² vg/hemisphere, or 1×10¹³ vg/hemisphere to about 5×10¹² vg/hemisphere). For example, the AAV vector is administered to the patient in an amount of about 1×10¹⁰ vg/hemisphere, about 5×10¹⁰ vg/hemisphere, or about 1×10¹¹ vg/hemisphere.

Upon administering the AAV vector including a transgene encoding hPGRN to the patient, the patient displays a change in PGRN levels. For example, the patient displays restoration of PGRN expression in the frontal cortex after administration of the AAV vector including a nucleic acid sequence encoding hPGRN to the patient. For example, the patient displays a level of PGRN expression in the frontal cortex of from about 2 ng/mg to about 8 ng/mg (e.g., 3 ng/mg to about 7 ng/mg, 4 ng/mg to about 6 ng/mg, or about 5 ng/mg), or more (e.g., about 9 ng/mg, about 10 ng/mg, about 15 ng/mg, about 20 ng/mg, about 30 ng/mg, about 40 ng/mg, about 50 ng/mg, about 60 ng/mg, about 70 ng/mg, about 80 ng/mg, about 90 ng/mg, or about 100 ng/mg). In addition to, or alternatively, for example, upon administering the AAV vector including a transgene encoding hPGRN to the patient, the patient displays an improvement in cognitive function.

### OTHER EMBODIMENTS

All publications, patents, and patent applications mentioned in this specification are incorporated herein by reference to the same extent as if each independent publication or patent application was specifically and individually indicated to be incorporated by reference.

While the invention has been described in connection with specific embodiments thereof, it will be understood that it is capable of further modifications and this application is intended to cover any variations, uses, or adaptations of the invention following, in general, the principles of the invention and including such departures from the invention that come within known or customary practice within the art to which the invention pertains and may be applied to the essential features hereinbefore set forth, and follows in the scope of the claims.

Other embodiments are within the claims.

1. A method of treating a disorder affecting the central nervous system (CNS) of a patient, the method comprising administering to the patient an adeno-associated viral (AAV) vector comprising a transgene encoding a therapeutic protein, wherein the AAV vector is administered to the patient intrathalamically in an amount of from about 1×10⁹ vg/hemisphere to about 9×10¹² vg/hemisphere.

2. A method of improving cognitive function in a patient diagnosed as having a disorder affecting the CNS of the patient, the method comprising administering to the patient an AAV vector comprising a transgene encoding a therapeutic protein, wherein the AAV vector is administered to the patient intrathalamically in an amount of from about 1×10⁹ vg/hemisphere to about 9×10¹² vg/hemisphere.

3. A method of expressing, in the brain (e.g., frontal cortex) of a patient diagnosed as having a disorder affecting the CNS, a therapeutic protein, the method comprising administering to the patient an AAV vector comprising a transgene encoding the therapeutic protein, wherein the AAV vector is administered to the patient intrathalamically in an amount of from about 1×10⁹ vg/hemisphere to about 9×10¹² vg/hemisphere.

4. The method of any one of claims 1-3, wherein the AAV vector is administered to the patient in an amount of from about 1×10¹⁰ vg/hemisphere to about 5×10¹² vg/hemisphere, optionally wherein the AAV vector is administered to the patient in an amount of about 1×10¹⁰ vg/hemisphere, 2×10¹⁰ vg/hemisphere, 3×10¹⁰ vg/hemisphere, 4×10¹⁰ vg/hemisphere, 5×10¹⁰ vg/hemisphere, 6×10¹⁰ vg/hemisphere, 7×10¹⁰ vg/hemisphere, 8×10¹⁰ vg/hemisphere, 9×10¹⁰ vg/hemisphere, 1×10¹¹ vg/hemisphere, 2×10¹¹ vg/hemisphere, 3×10¹¹ vg/hemisphere, 4×10¹¹ vg/hemisphere, 5×10¹¹ vg/hemisphere, 6×10¹¹ vg/hemisphere, 7×10¹¹ vg/hemisphere, 8×10¹¹ vg/hemisphere, 9×10¹¹ vg/hemisphere, 1×10¹² vg/hemisphere, 2×10¹² vg/hemisphere, 3×10¹² vg/hemisphere, 4×10¹² vg/hemisphere, or 5×10¹² vg/hemisphere.

5. The method of claim 4, wherein the AAV vector is administered to the patient in an amount of from about 5×10¹⁰ vg/hemisphere to about 9×10¹¹ vg/hemisphere.

6. The method of claim 5, wherein the AAV vector is administered to the patient in an amount of about 1×10¹⁰ vg/hemisphere.

7. The method of claim 5, wherein the AAV vector is administered to the patient in an amount of about 5×10¹⁰ vg/hemisphere.

8. The method of claim 5, wherein the AAV vector is administered to the patient in an amount of about 1×10¹¹ vg/hemisphere.

9. The method of any one of claims 1-8, wherein the AAV vector is administered to the patient in a single dose per hemisphere comprising the amount.

10. The method of any one of claims 1-8, wherein the AAV vector is administered to the patient in a plurality of doses per hemisphere that, together, comprise the amount.

11. The method of any one of claims 1-10, wherein the therapeutic protein is a protein whose deficiency or lack of activity is associated with the disorder.

12. The method of any one of claims 1-11, wherein the therapeutic protein is a secreted protein or a protein listed in Table 5 herein.

13. The method of any one of claims 1-12, wherein the therapeutic protein is PGRN.

14. The method of any one of claims 1-13, wherein the disorder is a neurocognitive disorder, a neuromuscular disorder, a neurodegenerative disorder, or a lysosomal storage disorder.

15. The method of any one of claims 1-13, wherein the disorder is a lysosomal storage disorder.

16. The method of any one of claims 1-13, wherein the disorder is frontotemporal dementia (FTD), Alzheimer's disease (AD), Parkinson's disease (PD), dementia with Lewy bodies, amyotrophic lateral sclerosis (ALS), or a related neurocognitive or motor neuron disorder.

17. The method of any one of claims 1-16, wherein the transgene is operably linked to a promoter that is active in a neuronal cell and/or a glial cell, optionally wherein the transgene encodes PGRN.

18. The method of claim 17, wherein the promoter is a synapsin promoter, a tetracycline-controlled transactivator protein (tTA) promoter, a reverse tetracycline-controlled transactivator protein (rTA) promoter, a U1 promoter, a U6 promoter, a U7 promoter, a prion promoter, a phosphoglycerate kinase (PGK) promoter, a CB7 promoter, an H1 promoter, a cytomegalovirus (CMV) promoter, a CMV-chicken β-actin (CBA) promoter, a glial fibrillary acidic protein (GFAP) promoter, a calcium/calmodulin-dependent protein kinase Ill promoter, a tubulin alpha I promoter, a microtubulin-associated protein IB (MAP IB) promoter, a neuron-specific enolase promoter, a platelet-derived growth factor beta chain promoter, a neurofilament light chain promoter, a neuron-specific VGF gene promoter, a neuronal nuclei (NeuN) promoter, a adenomatous polyposis coli (APC) promoter, an ionized calcium-binding adapter molecule 1 (Iba-1) promoter, or a homeobox protein 9 (HB9) promoter.

19. The method of claim 18, wherein the promoter is a synapsin promoter.

20. The method of claim 19, wherein the synapsin promoter has a nucleic acid sequence that is at least 85% identical to the nucleic acid sequence of SEQ ID NO: 1.

21. The method of claim 20, wherein the synapsin promoter has a nucleic acid sequence that is at least 90% identical to the nucleic acid sequence of SEQ ID NO: 1, optionally wherein the synapsin promoter has a nucleic acid sequence that is at least 95%, 96%, 97%, 98%, or 99% identical to the nucleic acid sequence of SEQ ID NO: 1.

22. The method of claim 21, wherein the synapsin promoter has the nucleic acid sequence of SEQ ID NO: 1.

23. The method of any one of claims 1-22, wherein the therapeutic protein is PGRN, and wherein the PGRN has an amino acid sequence that is at least 85% identical to the amino acid sequence of SEQ ID NO: 2.

24. The method of claim 23, wherein the PGRN has an amino acid sequence that is at least 90% identical to the amino acid sequence of SEQ ID NO: 2, optionally wherein the PGRN has an amino acid sequence that is at least 95%, 96%, 97%, 98%, or 99% identical to the amino acid sequence of SEQ ID NO: 2.

25. The method of claim 24, wherein the PGRN has the amino acid sequence of SEQ ID NO: 2.

26. The method of any one of claims 1-25, wherein the transgene encodes PGRN, and wherein the transgene encoding PGRN is codon-optimized.

27. The method of claim 26, wherein the transgene encoding PGRN has a nucleic acid sequence that is at least 85% identical to the nucleic acid sequence of SEQ ID NO: 3.

28. The method of claim 27, wherein the transgene encoding PGRN has a nucleic acid sequence that is at least 90% identical to the nucleic acid sequence of SEQ ID NO: 3, optionally wherein the transgene encoding PGRN has a nucleic acid sequence that is at least 95%, 96%, 97%, 98%, or 99% identical to the nucleic acid sequence of SEQ ID NO: 3.

29. The method of claim 28, wherein the transgene encoding PGRN has the nucleic acid sequence of SEQ ID NO: 3.

30. The method of any one of claims 1-29, wherein the transgene is operably linked to a human growth hormone (hGH) intron, optionally wherein: (i) the hGH intron is an hGH intron 3 and/or (ii) the transgene encodes PGRN.

31. The method of claim 30, wherein the hGH intron has a nucleic acid sequence that is at least 85% identical to the nucleic acid sequence of SEQ ID NO: 4.

32. The method of claim 31, wherein the hGH intron has a nucleic acid sequence that is at least 90% identical to the nucleic acid sequence of SEQ ID NO: 4, optionally wherein the hGH intron has a nucleic acid sequence that is at least 95%, 96%, 97%, 98%, or 99% identical to the nucleic acid sequence of SEQ ID NO: 4.

33. The method of claim 32, wherein the hGH intron has the nucleic acid sequence of SEQ ID NO: 4.

34. The method of any one of claims 1-33, wherein the transgene is operably linked to a 3′ enhancer element, optionally wherein the transgene encodes PGRN.

35. The method of claim 34, wherein the 3′ enhancer element has a nucleic acid sequence that is at least 85% identical to the nucleic acid sequence of SEQ ID NO: 5.

36. The method of claim 35, wherein the 3′ enhancer element has a nucleic acid sequence that is at least 90% identical to the nucleic acid sequence of SEQ ID NO: 5, optionally wherein the 3′ enhancer element has a nucleic acid sequence that is at least 95%, 96%, 97%, 98%, or 99% identical to the nucleic acid sequence of SEQ ID NO: 5.

37. The method of claim 36, wherein the 3′ enhancer element has the nucleic acid sequence of SEQ ID NO: 5.

38. The method of any one of claims 1-37, wherein the AAV vector comprises capsid proteins from an AAV serotype selected from the group consisting of AAV1, AAV2, AAV3, AAV4, AAV5, AAV6, AAV7, AAV8, AAV9, AAVrh74, AAVrh,8, and AAVrh, 10.

39. The method of any one of claims 1-38, wherein the AAV is an anterogradely-trafficked AAV or a retrogradely-trafficked AAV.

40. The method of any one of claims 1-39, wherein the AAV vector comprises a 5′ inverted terminal repeat (ITR) and/or a 3′ ITR from AAV1, AAV2, AAV3, AAV4, AAV5, AAV6, AAV7, AAV8, AAV9, AAVrh74, AAVrh,8, or AAVrh, 10, optionally wherein the AAV vector comprises a 5′ ITR and a 3′ ITR from AAV2.

41. The method of any one of claims 1-40, wherein the AAV vector comprises a 5′ ITR and a 3′ ITR from one AAV serotype and capsid proteins from a different AAV serotype.

42. The method of any one of claims 1-41, wherein the AAV vector is an AAV2/9 vector.

43. The method of any one of claims 1-42, wherein the AAV has a nucleic acid sequence that is at least 85% identical to the nucleic acid sequence of SEQ ID NO: 6.

44. The method of claim 43, wherein the AAV has a nucleic acid sequence that is at least 90% identical to the nucleic acid sequence of SEQ ID NO: 6.

45. The method of claim 44, wherein the AAV has a nucleic acid sequence that is at least 95% identical to the nucleic acid sequence of SEQ ID NO: 6, optionally wherein the AAV has a nucleic acid sequence that is at least 96%, 97%, 98%, or 99% identical to the nucleic acid sequence of SEQ ID NO: 6.

46. The method of claim 45, wherein the AAV has a nucleic acid of SEQ ID NO: 6.

47. The method of any one of claims 1-46, wherein the therapeutic protein is PGRN, and wherein, prior to administration of the AAV vector, the patient exhibits a level of expression of endogenous PGRN that is from about 1% to about 40% of the level of endogenous PGRN expression observed in a human subject of the same age, gender, and/or body mass index that does not have FTD, AD, PD, dementia with Lewy bodies, ALS, or a related motor neuron disorder.

48. The method of any one of claims 1-47, wherein the therapeutic protein is PGRN, and wherein, following administration of the AAV vector, the patient exhibits an increase in PGRN expression relative to a measurement of the patient's PGRN expression level obtained prior to administration of the AAV vector.

49. The method of claim 48, wherein the increase in PGRN expression is observed in the patient's thalamus, frontal cortex, basal ganglia, parietal cortex, temporal cortex, parietal and temporal cortices, and/or cerebral spinal fluid (CSF).

50. The method of any one of claims 1-49, wherein the therapeutic protein is PGRN, and wherein, following administration of the AAV vector, the patient exhibits a level of PGRN expression of from about 2 ng/mg to about 100 ng/mg in the frontal cortex.

51. The method of any one of claims 1-50, wherein the AAV vector is administered to the patient in a convection-assisted manner.

52. A method of treating a disorder affecting the CNS of a patient, the method comprising administering to the patient an AAV vector comprising a transgene encoding a therapeutic protein, wherein the AAV vector is administered to the patient in an amount sufficient to achieve a level of therapeutic protein expression in the brain (e.g., frontal cortex) of the patient that is equivalent to a level of therapeutic protein expression observed in a human subject having the disorder following intrathalamic administration, in an amount of from about 1×10⁹ vg/hemisphere to about 9×10¹² vg/hemisphere, of an AAV2/9 vector comprising a transgene encoding the therapeutic protein.

53. A method of improving cognitive function in a human patient diagnosed as having a disorder affecting the CNS of a patient, the method comprising administering to the patient an AAV vector comprising a transgene encoding a therapeutic protein, wherein the AAV vector is administered to the patient in an amount sufficient to achieve a level of therapeutic protein expression in the brain (e.g., frontal cortex) of the patient that is equivalent to a level of therapeutic protein expression observed in a human subject having the disorder following intrathalamic administration, in an amount of from about 1×10⁹ vg/hemisphere to about 9×10¹² vg/hemisphere, of an AAV2/9 vector comprising a transgene encoding the therapeutic protein.

54. A method of expressing, in the brain (e.g., frontal cortex) of a human patient diagnosed as having a disorder affecting the CNS of the patient, a therapeutic protein, the method comprising administering to the patient an AAV vector comprising a transgene encoding the therapeutic protein, wherein the AAV vector is administered to the patient in an amount sufficient to achieve a level of therapeutic protein expression in the brain (e.g., frontal cortex) of the patient that is equivalent to a level of therapeutic protein expression observed in a human subject having the disorder following intrathalamic administration, in an amount of from about 1×10⁹ vg/hemisphere to about 9×10¹² vg/hemisphere, of an AAV2/9 vector comprising a transgene encoding the therapeutic protein.

55. A method of treating FTD, AD, PD, dementia with Lewy bodies, a related neurocognitive disorder, ALS, or a related motor neuron disorder in a human patient in need thereof, the method comprising administering to the patient an AAV vector comprising a transgene encoding PGRN, wherein the AAV vector is administered to the patient in an amount sufficient to achieve a level of PGRN expression in the brain (e.g., frontal cortex) of the patient of from about 2 ng/mg to about 8 ng/mg, or more.

56. A method of improving cognitive function in a human patient diagnosed as having FTD, AD, PD, dementia with Lewy bodies, a related neurocognitive disorder, ALS, or a related motor neuron disorder, the method comprising administering to the patient an AAV vector comprising a transgene encoding PGRN, wherein the AAV vector is administered to the patient in an amount sufficient to achieve a level of PGRN expression in the brain (e.g., frontal cortex) of the patient of from about 2 ng/mg to about 8 ng/mg, or more.

57. A method of expressing PGRN in the brain (e.g., frontal cortex) of a human patient diagnosed as having FTD, AD, PD, dementia with Lewy bodies, a related neurocognitive disorder, ALS, or a related motor neuron disorder, the method comprising administering to the patient an AAV vector comprising a transgene encoding PGRN, wherein the AAV vector is administered to the patient in an amount sufficient to achieve a level of PGRN expression in the brain (e.g., frontal cortex) of the patient of from about 2 ng/mg to about 8 ng/mg, or more.

58. The method of any one of claims 52-54, wherein the therapeutic protein is a protein whose deficiency or lack of activity is associated with the disorder.

59. The method of any one of claims 52-54, wherein the therapeutic protein is a secreted protein or a protein listed in Table 5 herein.

60. The method of any one of claims 52-54, wherein the therapeutic protein is PGRN.

61. The method of any one of claims 52-54, wherein the disorder is a neurocognitive disorder, a neuromuscular disorder, a neurodegenerative disorder, or a lysosomal storage disorder.

62. The method of any one of claims 52-54, wherein the disorder is a lysosomal storage disorder.

63. The method of any one of claims 52-54, wherein the disorder is frontotemporal dementia (FTD), Alzheimer's disease (AD), Parkinson's disease (PD), dementia with Lewy bodies, amyotrophic lateral sclerosis (ALS), or a related neurocognitive or motor neuron disorder.

64. The method of any one of claims 52-54, wherein the AAV2/9 vector comprising a transgene encoding the therapeutic protein has the nucleic acid sequence of SEQ ID NO: 6.

65. The method of any one of claims 52-64, wherein the AAV vector administered to the patient is an anterogradely-trafficked AAV or a retrogradely-trafficked AAV.

66. The method of claim 65, wherein the AAV vector administered to the patient comprises capsid proteins from an AAV serotype selected from the group consisting of AAV1, AAV2, AAV3, AAV4, AAV5, AAV6, AAV7, AAV8, AAV9, AAVrh74, AAVrh,8, and AAVrh, 10.

67. The method of any one of claims 52-66, wherein the AAV vector administered to the patient comprises a 5′ ITR and/or a 3′ ITR from AAV1, AAV2, AAV3, AAV4, AAV5, AAV6, AAV7, AAV8, AAV9, AAVrh74, AAVrh,8, or AAVrh, 10, optionally wherein the AAV vector administered to the patient comprises a 5′ ITR and a 3′ ITR from AAV2.

68. The method of any one of claims 52-67, wherein the AAV vector administered to the patient comprises a 5′ ITR and a 3′ ITR from one AAV serotype and capsid proteins from a different AAV serotype.

69. The method of any one of claims 52-68, wherein the AAV vector administered to the patient is an AAV2/9 vector.

70. The method of any one of claims 1-69, wherein the human patient is diagnosed as having FTD due to a mutation in the GRN gene.

71. The method of any one of claims 1-70, wherein upon administration of the AAV vector, there is an observed increase of transgene expression in the peripheral tissues of less than 10%.

72. The method of claim 71, wherein the peripheral tissues include the liver, lung, and/or spleen.

73. The method of claim 71 or 72, wherein the transgene expression is calculated relative to GAPDH expression.

74. The method of any one of claims 1-73, wherein the patient is a mammal.

75. The method of any one of claims 1-74, wherein the patient is a human.

76. An adeno-associated viral (AAV) vector for use in treating a disorder affecting the central nervous system (CNS) of a patient, wherein the AAV vector comprises a transgene encoding a therapeutic protein, and wherein the AAV vector is administered to the patient intrathalamically in an amount of from about 1×10⁹ vg/hemisphere to about 9×10¹² vg/hemisphere.

77. The AAV for use according to claim 76, wherein the AAV vector is administered to the patient in an amount of from about 1×10¹⁰ vg/hemisphere to about 5×10¹² vg/hemisphere, optionally wherein the AAV vector is administered to the patient in an amount of about 1×10¹⁰ vg/hemisphere, 2×10¹⁰ vg/hemisphere, 3×10¹⁰ vg/hemisphere, 4×10¹⁰ vg/hemisphere, 5×10¹⁰ vg/hemisphere, 6×10¹⁰ vg/hemisphere, 7×10¹⁰ vg/hemisphere, 8×10¹⁰ vg/hemisphere, 9×10¹⁰ vg/hemisphere, 1×10¹¹ vg/hemisphere, 2×10¹¹ vg/hemisphere, 3×10¹¹ vg/hemisphere, 4×10¹¹ vg/hemisphere, 5×10¹¹ vg/hemisphere, 6×10¹¹ vg/hemisphere, 7×10¹¹ vg/hemisphere, 8×10¹¹ vg/hemisphere, 9×10¹¹ vg/hemisphere, 1×10¹² vg/hemisphere, 2×10¹² vg/hemisphere, 3×10¹² vg/hemisphere, 4×10¹² vg/hemisphere, or 5×10¹² vg/hemisphere.

78. The AAV for use according to claim 76, wherein the AAV vector is administered to the patient in an amount of from about 5×10¹⁰ vg/hemisphere to about 9×10¹¹ vg/hemisphere.

79. The AAV for use according to claim 76, wherein the AAV vector is administered to the patient in an amount of about 1×10¹⁰ vg/hemisphere.

80. The AAV for use according to claim 76, wherein the AAV vector is administered to the patient in an amount of about 5×10¹⁰ vg/hemisphere.

81. The AAV for use according to claim 76, wherein the AAV vector is administered to the patient in an amount of about 1×10¹¹ vg/hemisphere.

82. The AAV for use according to claim 76, wherein the AAV vector is administered to the patient in a single dose per hemisphere comprising the amount.

83. The AAV for use according to claim 76, wherein the AAV vector is administered to the patient in a plurality of doses per hemisphere that, together, comprise the amount.

84. The AAV for use according to claim 76, wherein the therapeutic protein is a protein whose deficiency or lack of activity is associated with the disorder.

85. The AAV for use according to claim 76, wherein the therapeutic protein is a secreted protein or a protein listed in Table 5 herein.

86. The AAV for use according to claim 76, wherein the therapeutic protein is PGRN.

87. The AAV for use according to claim 76, wherein the disorder is a neurocognitive disorder, a neuromuscular disorder, a neurodegenerative disorder, or a lysosomal storage disorder.

88. The AAV for use according to claim 76, wherein the disorder is a lysosomal storage disorder.

89. A kit comprising an AAV vector comprising a transgene encoding PGRN, wherein the kit further comprises a package insert instructing a user of the kit to administer the AAV vector to the patient in accordance with the method of any one of claims 1-75.


SEQUENCE LISTING (10 sequences)

SEQ ID NO: 1 - DNA, 448 bp, synthetic construct
  source 1..448: mol_type = other DNA
agtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcag

SEQ ID NO: 2 - protein, 593 aa, synthetic construct
MWTLVSWVALTAGLVAGTRCPDGQFCPVACCLDPGGASYSCCRPLLDKWPTTLSRHLGGPCQVDAHCSAGHSCIFTVSGTSSCCPFPEAVACGDGHHCCPRGFHCSADGRSCFQRSGNNSVGAIQCPDSQFECPDFSTCCVMVDGSWGCCPMPQASCCEDRVHCCPHGAFCDLVHTRCITPTGTHPLAKKLPAQRTNRAVALSSSVMCPDARSRCPDGSTCCELPSGKYGCCPMPNATCCSDHLHCCPQDTVCDLIQSKCLSKENATTDLLTKLPAHTVGDVKCDMEVSCPDGYTCCRLQSGAWGCCPFTQAVCCEDHIHCCPAGFTCDTQKGTCEQGPHQVPWMEKAPAHLSLPDPQALKRDVPCDNVSSCPSSDTCCQLTSGEWGCCPIPEAVCCSDHQHCCPQGYTCVAEGQCQRGSEIVAGLEKMPARRASLSHPRDIGCDQHTSCPVGQTCCPSLGGSWACCQLPHAVCCEDRQHCCPAGYTCNVKARSCEKEVVSAQPATFLARSPHVGVKDVECGEGHFCHDNQTCCRDNRQGWACCPYRQGVCCADRRHCCPAGFRCAARGTKCLRREAPRWDAPLRDPALRQLL

SEQ ID NO: 3 - DNA, 1782 bp, synthetic construct
  source 1..1782: mol_type = other DNA
atgtggactctggtctcctgggtcgctctgaccgctggcctggtcgctgggacaagatgccccgatggacagttttgccccgtcgcttgctgtctggacccaggaggagccagctactcctgctgtcggccactgctggataagtggcccaccacactgtcccgccacctgggaggaccatgccaggtggacgcacactgttccgccggacactcttgcatcttcacagtgtctggcaccagctcctgctgtccatttcctgaggcagtggcatgcggcgacggacaccactgctgtcccaggggcttccactgtagcgccgatggcaggtcctgctttcagagaagcggcaacaattccgtgggcgccatccagtgtcctgacagccagttcgaatgcccagatttttccacctgctgcgtgatggtggacggctcttggggctgctgtccaatgccacaggccagctgctgtgaggacagggtgcactgctgtcctcacggagccttctgtgatctggtgcacacacgctgcatcacccccacaggcacccaccctctggccaagaagctgccagcacagaggaccaacagggcagtggccctgagcagcagcgtgatgtgccccgacgccaggtctagatgccctgatggcagcacctgctgtgagctgccaagcggcaagtacggctgctgtcctatgccaaacgccacatgctgttccgaccacctgcactgctgtcctcaggacaccgtgtgcgatctgatccagtctaagtgcctgagcaaggagaatgccaccacagacctgctgacaaagctgcctgcccacaccgtgggcgacgtgaagtgtgatatggaggtgtcctgcccagatggctatacatgctgtaggctgcagtctggagcatggggatgctgtcccttcacccaggccgtgtgctgtgaggaccacatccactgctgtcctgccggctttacatgtgatacccagaagggcacatgcgagcagggccctcaccaggtgccatggatggagaaggcaccagcacacctgtccctgcccgaccctcaggccctgaagagagacgtgccttgtgataacgtgtctagctgcccatcctctgatacatgctgtcagctgacctctggcgagtggggctgctgtccaatccccgaggccgtgtgctgtagcgaccaccagcactgctgtcctcagggctatacctgcgtggcagagggacagtgccagaggggctccgagatcgtggcaggcctggagaagatgccagccaggagagcctctctgagccaccccagagacatcggctgtgatcagcacacaagctgcccagtgggacagacctgctgtccatccctgggaggctcttgggcatgctgtcagctgcctcacgccgtgtgctgtgaggataggcagcactgctgtccagccggctacacatgcaatgtgaaggccagatcctgcgagaaggaggtggtgtctgcccagccagccaccttcctggcacgcagccctcacgtgggcgtgaaggacgtggagtgtggcgagggccacttttgccacgacaaccagacatgctgtagggataatagacagggctgggcctgctgtccatataggcagggcgtgtgctgtgcagatcggcgccactgctgtccagcaggctttcggtgcgcagccaggggcaccaagtgcctgcgcagagaagccccccggtgggacgcccccctgcgagaccccgccctgagacagctgctgtga

SEQ ID NO: 4 - DNA, 92 bp, synthetic construct
  source 1..92: mol_type = other DNA
gtgagtggatgccttctccccaggcggggatgggggagacctgtagtcagagcccccgggcagcacagccaatgcccgtccttcccctgcag

SEQ ID NO: 5 - DNA, 284 bp, synthetic construct
  source 1..284: mol_type = other DNA
gggacagtactgaagactctgcagccctcgggaccccactcggagggtgccctctgctcaggcctccctagcacctccccctaaccaaattctccctggaccccattctgagctccccatcaccatgggaggtggggcctcaatctaaggccttccctgtcagaagggggttgtggcaaaagccacattacaagctgccatcccctccccgtttcagtggaccctgtggccaggtgcttttccctatccacaggggtgtttgtgtgtgtgcgcgtgtgcgtttc

SEQ ID NO: 6 - DNA, 5946 bp, synthetic construct
  source 1..5946: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgttgcaaagatggataaagttttaaacagagaggaatctttgcagctaatggaccttctaggtcttgaaaggagtgggaattggctccggtgcccgtcagtgggcagagcgcacatcgcccacagtccccgagaagttggggggaggggtcggcagcaaatggttaattaatctagactgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagtcgagaggatccgtgagtggatgccttctccccaggcggggatgggggagacctgtagtcagagcccccgggcagcacagccaatgcccgtccttcccctgcagaccggtgccaccatgtggactctggtctcctgggtcgctctgaccgctggcctggtcgctgggacaagatgccccgatggacagttttgccccgtcgcttgctgtctggacccaggaggagccagctactcctgctgtcggccactgctggataagtggcccaccacactgtcccgccacctgggaggaccatgccaggtggacgcacactgttccgccggacactcttgcatcttcacagtgtctggcaccagctcctgctgtccatttcctgaggcagtggcatgcggcgacggacaccactgctgtcccaggggcttccactgtagcgccgatggcaggtcctgctttcagagaagcggcaacaattccgtgggcgccatccagtgtcctgacagccagttcgaatgcccagatttttccacctgctgcgtgatggtggacggctcttggggctgctgtccaatgccacaggccagctgctgtgaggacagggtgcactgctgtcctcacggagccttctgtgatctggtgcacacacgctgcatcacccccacaggcacccaccctctggccaagaagctgccagcacagaggaccaacagggcagtggccctgagcagcagcgtgatgtgccccgacgccaggtctagatgccctgatggcagcacctgctgtgagctgccaagcggcaagtacggctgctgtcctatgccaaacgccacatgctgttccgaccacctgcactgctgtcctcaggacaccgtgtgcgatctgatccagtctaagtgcctgagcaaggagaatgccaccacagacctgctgacaaagctgcctgcccacaccgtgggcgacgtgaagtgtgatatggaggtgtcctgcccagatggctatacatgctgtaggctgcagtctggagcatggggatgctgtcccttcacccaggccgtgtgctgtgaggaccacatccactgctgtcctgccggctttacatgtgatacccagaagggcacatgcgagcagggccctcaccaggtgccatggatggagaaggcaccagcacacctgtccctgcccgaccctcaggccctgaagagagacgtgccttgtgataacgtgtctagctgcccatcctctgatacatgctgtcagctgacctctggcgagtggggctgctgtccaatccccgaggccgtgtgctgtagcgaccaccagcactgctgtcctcagggctatacctgcgtggcagagggacagtgccagaggggctccgagatcgtggcaggcctggagaagatgccagccaggagagcctctctgagccaccccagagacatcggctgtgatcagcacacaagctgcccagtgggacagacctgctgtccatccctgggaggctcttgggcatgctgtcagctgcctcacgccgtgtgctgtgaggataggcagcactgctgtccagccggctacacatgcaatgtgaaggccagatcctgcgagaaggaggtggtgtctgcccagccagccaccttcctggcacgcagccctcacgtgggcgtgaaggacgtggagtgtggcgagggccacttttgccacgacaaccagacatgctgtagggataatagacagggctgggcctgctgtccatataggcagggcgtgtgctgtgcagatcggcgccactgctgtccagcaggctttcggtgcgcagccaggggcaccaagtgcctgcgcagagaagccccccggtgggacgcccccctgcgagaccccgccctgagacagctgctgtgagtcgctggtttaaacgggacagtactgaagactctgcagccctcgggaccccactcggagggtgccctctgctcaggcctccctagcacctccccctaaccaaattctccctggaccccattctgagctccccatcaccatgggaggtggggcctcaatctaaggccttccctgtcagaagggggttgtggcaaaagccacattacaagctgccatcccctccccgtttcagtggaccctgtggccaggtgcttttccctatccacaggggtgtttgtgtgtgtgcgcgtgtgcgtttcgctagcctcgagagatcgatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggacacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgccactagttgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaataactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcaggcctacatgt

SEQ ID NO: 7 - DNA, 1781 bp, synthetic construct
  source 1..1781: mol_type = other DNA
tgtggaccctggtgagctgggtggccttaacagcagggctggtggctggaacgcggtgcccagatggtcagttctgccctgtggcctgctgcctggaccccggaggagccagctacagctgctgccgtccccttctggacaaatggcccacaacactgagcaggcatctgggtggcccctgccaggttgatgcccactgctctgccggccactcctgcatctttaccgtctcagggacttccagttgctgccccttcccagaggccgtggcatgcggggatggccatcactgctgcccacggggcttccactgcagtgcagacgggcgatcctgcttccaaagatcaggtaacaactccgtgggtgccatccagtgccctgatagtcagttcgaatgcccggacttctccacgtgctgtgttatggtcgatggctcctgggggtgctgccccatgccccaggcttcctgctgtgaagacagggtgcactgctgtccgcacggtgccttctgcgacctggttcacacccgctgcatcacacccacgggcacccaccccctggcaaagaagctccctgcccagaggactaacagggcagtggccttgtccagctcggtcatgtgtccggacgcacggtcccggtgccctgatggttctacctgctgtgagctgcccagtgggaagtatggctgctgcccaatgcccaacgccacctgctgctccgatcacctgcactgctgcccccaagacactgtgtgtgacctgatccagagtaagtgcctctccaaggagaacgctaccacggacctcctcactaagctgcctgcgcacacagtgggggatgtgaaatgtgacatggaggtgagctgcccagatggctatacctgctgccgtctacagtcgggggcctggggctgctgcccttttacccaggctgtgtgctgtgaggaccacatacactgctgtcccgcggggtttacgtgtgacacgcagaagggtacctgtgaacaggggccccaccaggtgccctggatggagaaggccccagctcacctcagcctgccagacccacaagccttgaagagagatgtcccctgtgataatgtcagcagctgtccctcctccgatacctgctgccaactcacgtctggggagtggggctgctgtccaatcccagaggctgtctgctgctcggaccaccagcactgctgcccccagggctacacgtgtgtagctgaggggcagtgtcagcgaggaagcgagatcgtggctggactggagaagatgcctgcccgccgggcttccttatcccaccccagagacatcggctgtgaccagcacaccagctgcccggtggggcagacctgctgcccgagcctgggtgggagctgggcctgctgccagttgccccatgctgtgtgctgcgaggatcgccagcactgctgcccggctggctacacctgcaacgtgaaggctcgatcctgcgagaaggaagtggtctctgcccagcctgccaccttcctggcccgtagccctcacgtgggtgtgaaggacgtggagtgtggggaaggacacttctgccatgataaccagacctgctgccgagacaaccgacagggctgggcctgctgtccctaccgccagggcgtctgttgtgctgatcggcgccactgctgtcctgctggcttccgctgcgcagccaggggtaccaagtgtttgcgcagggaggccccgcgctgggacgcccctttgagggacccagccttgagacagctgctgtga

SEQ ID NO: 8 - DNA, 208 bp, synthetic construct
  source 1..208: mol_type = other DNA
ctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctgggga

SEQ ID NO: 9 - DNA, 256 bp, synthetic construct
  source 1..256: mol_type = other DNA
attctccaatcacatgatccctagaaatggggtgtggggcgagaggaagcagggaggagagtgatttgagtagaaaagaaacacagcattccaggctggccccacctctatattgataagtagccaatgggagcgggtagccctgatccctggccaatggaaactgaggtaggcgggtcatcgcgctggggtctgtagtctgagcgctacccggttgctgctgcccaaggaccgcggagtcggacgcaggcagacc

SEQ ID NO: 10 - DNA, 2356 bp, synthetic construct
  source 1..2356: mol_type = other DNA
attctccaatcacatgatccctagaaatggggtgtggggcgagaggaagcagggaggagagtgatttgagtagaaaagaaacacagcattccaggctggccccacctctatattgataagtagccaatgggagcgggtagccctgatccctggccaatggaaactgaggtaggcgggtcatcgcgctggggtctgtagtctgagcgctacccggttgctgctgcccaaggaccgcggagtcggacgcaggcagaccatgtggaccctggtgagctgggtggccttaacagcagggctggtggctggaacgcggtgcccagatggtcagttctgccctgtggcctgctgcctggaccccggaggagccagctacagctgctgccgtccccttctggacaaatggcccacaacactgagcaggcatctgggtggcccctgccaggttgatgcccactgctctgccggccactcctgcatctttaccgtctcagggacttccagttgctgccccttcccagaggccgtggcatgcggggatggccatcactgctgcccacggggcttccactgcagtgcagacgggcgatcctgcttccaaagatcaggtaacaactccgtgggtgccatccagtgccctgatagtcagttcgaatgcccggacttctccacgtgctgtgttatggtcgatggctcctgggggtgctgccccatgccccaggcttcctgctgtgaagacagggtgcactgctgtccgcacggtgccttctgcgacctggttcacacccgctgcatcacacccacgggcacccaccccctggcaaagaagctccctgcccagaggactaacagggcagtggccttgtccagctcggtcatgtgtccggacgcacggtcccggtgccctgatggttctacctgctgtgagctgcccagtgggaagtatggctgctgcccaatgcccaacgccacctgctgctccgatcacctgcactgctgcccccaagacactgtgtgtgacctgatccagagtaagtgcctctccaaggagaacgctaccacggacctcctcactaagctgcctgcgcacacagtgggggatgtgaaatgtgacatggaggtgagctgcccagatggctatacctgctgccgtctacagtcgggggcctggggctgctgcccttttacccaggctgtgtgctgtgaggaccacatacactgctgtcccgcggggtttacgtgtgacacgcagaagggtacctgtgaacaggggccccaccaggtgccctggatggagaaggccccagctcacctcagcctgccagacccacaagccttgaagagagatgtcccctgtgataatgtcagcagctgtccctcctccgatacctgctgccaactcacgtctggggagtggggctgctgtccaatcccagaggctgtctgctgctcggaccaccagcactgctgcccccagggctacacgtgtgtagctgaggggcagtgtcagcgaggaagcgagatcgtggctggactggagaagatgcctgcccgccgggcttccttatcccaccccagagacatcggctgtgaccagcacaccagctgcccggtggggcagacctgctgcccgagcctgggtgggagctgggcctgctgccagttgccccatgctgtgtgctgcgaggatcgccagcactgctgcccggctggctacacctgcaacgtgaaggctcgatcctgcgagaaggaagtggtctctgcccagcctgccaccttcctggcccgtagccctcacgtgggtgtgaaggacgtggagtgtggggaaggacacttctgccatgataaccagacctgctgccgagacaaccgacagggctgggcctgctgtccctaccgccagggcgtctgttgtgctgatcggcgccactgctgtcctgctggcttccgctgcgcagccaggggtaccaagtgtttgcgcagggaggccccgcgctgggacgcccctttgagggacccagccttgagacagctgctgtgagggacagtactgaagactctgcagccctcgggaccccactcggagggtgccctctgctcaggcctccctagcacctccccctaaccaaattctccctggaccccattctgagctccccatcaccatgggaggtggggcctcaatctaaggccttccctgtcagaagggggttgtggcaaaagccacattacaagctgccatcccctccccgtttcagtggaccctgtggccaggtgcttttccctatccacaggggtgtttgtgtgtgtgcgcgtgtgcgtttcaataaagtttgtacactttcttaaaaaaaaaaaa